SEQUENCE LISTING (21 sequences)

SEQ ID NO: 1 - protein, 40 aa, Artificial Sequence
  Synthetic
  MOD_RES (2)..(2): Aib
  MOD_RES (20)..(20): Aib
YXEGTFTSDYSIYLDKQAAXEFVNWLLAGGPSSGAPPPSK

SEQ ID NO: 2 - protein, 39 aa, Artificial Sequence
  Synthetic
  MOD_RES (2)..(2): Aib
  MOD_RES (20)..(20): Aib
YXEGTFTSDYSIYLDKKAAXEFVNWLLAGGPSSGAPPPS

SEQ ID NO: 3 - protein, 39 aa, Artificial Sequence
  Synthetic
  MOD_RES (2)..(2): Aib
  MOD_RES (20)..(20): Aib
YXEGTFTSDYSIYLDKQAAXKFVNWLLAGGPSSGAPPPS

SEQ ID NO: 4 - protein, 39 aa, Artificial Sequence
  Synthetic
  MISC_FEATURE (1)..(1): Xaa is Ac-D-Tyr
  MOD_RES (20)..(20): Aib
XAEGTFTSDYSIYLDKQAAXKFVNWLLAGGPSSGAPPPS

SEQ ID NO: 5 - protein, 39 aa, Artificial Sequence
  Synthetic
  MOD_RES (2)..(2): Aib
  MOD_RES (20)..(20): Aib
YXEGTFISDYSIYLDKQAAXKFVNWLLAGGPSSGAPPPS

SEQ ID NO: 6 - protein, 39 aa, Artificial Sequence
  Synthetic
  MOD_RES (2)..(2): Aib
  MOD_RES (20)..(20): Aib
HXEGTFTSDVSIYLDKQAAXKFVNWLLAGGPSSGAPPPS

SEQ ID NO: 7 - protein, 39 aa, Artificial Sequence
  Synthetic
  MOD_RES (2)..(2): Aib
  MOD_RES (20)..(20): Aib
YXEGTFTSDYSIYLEKQAAXKFVNWLLAGGPSSGAPPPS

SEQ ID NO: 8 - protein, 35 aa, Artificial Sequence
  Synthetic
  MOD_RES (2)..(2): Aib
  MOD_RES (20)..(20): Aib
YXEGTFTSDYSIYLEKQAAXKFVNWLLAGGPSSGA

SEQ ID NO: 9 - protein, 39 aa, Artificial Sequence
  Synthetic
  MOD_RES (2)..(2): Aib
  MOD_RES (20)..(20): Aib
YXEGTFTSDYSIYLEEQAAXKFVNWLLAGGPSSGAPPPS

SEQ ID NO: 10 - protein, 39 aa, Artificial Sequence
  Synthetic
  MOD_RES (2)..(2): Aib
  MOD_RES (20)..(20): Aib
YXEGTFTSDYSIYLEKQAAXEFVNWLLAGGPSSGAPPPS

SEQ ID NO: 11 - protein, 39 aa, Artificial Sequence
  Synthetic
  MOD_RES (2)..(2): Aib
  MOD_RES (20)..(20): Aib
YXEGTFTSDYSIYLEKKAAXEFVNWLLAGGPSSGAPPPS

SEQ ID NO: 12 - protein, 39 aa, Artificial Sequence
  Synthetic
  MOD_RES (2)..(2): Aib
  MOD_RES (20)..(20): Aib
YXEGTFTSDYSIYLEKQAAXKFVQWLLEGGPSSGAPPPS

SEQ ID NO: 13 - protein, 35 aa, Artificial Sequence
  Synthetic
  MOD_RES (2)..(2): Aib
  MOD_RES (20)..(20): Aib
YXEGTFTSDYSIYLEKQAAXEFVQWLLEGGPSSGA

SEQ ID NO: 14 - protein, 39 aa, Artificial Sequence
  Synthetic
  MOD_RES (2)..(2): Aib
  MOD_RES (20)..(20): Aib
YXEGTFTSDYSIYLEKKAAXEFVQWLLEGGPSSGAPPPS

SEQ ID NO: 15 - protein, 39 aa, Artificial Sequence
  Synthetic sequence
  MOD_RES (2)..(2): Aib
  MISC_FEATURE (15)..(15): Xaa is Asp  or Glu
  MISC_FEATURE (16)..(16): Xaa is Glu or Lys
  MISC_FEATURE (17)..(17): Xaa is Gln or Lys
  MOD_RES (20)..(20): Aib
  MISC_FEATURE (21)..(21): Xaa is Glu or Lys
  MISC_FEATURE (24)..(24): Xaa is Asn or Gln
  MISC_FEATURE (28)..(28): Xaa is Ala or Glu
  MISC_FEATURE (32)..(32): Xaa is Ser or absent
  MISC_FEATURE (33)..(33): Xaa is Ser or absent
  MISC_FEATURE (34)..(34): Xaa is Gly or absent
  MISC_FEATURE (35)..(35): Xaa is Ala or absent
  MISC_FEATURE (36)..(36): Xaa is Pro or absent
  MISC_FEATURE (37)..(37): Xaa is Pro or absent
  MISC_FEATURE (38)..(38): Xaa is Pro or absent
  MISC_FEATURE (39)..(39): Xaa is Ser or absent
YXEGTFTSDYSIYLXXXAAXXFVXWLLXGGPXXXXXXXX

SEQ ID NO: 16 - protein, 35 aa, Artificial Sequence
  Synthetic
  MOD_RES (2)..(2): Aib
  MISC_FEATURE (15)..(15): Xaa is Asp or Glu
  MISC_FEATURE (16)..(16): Xaa is Glu or Lys
  MISC_FEATURE (17)..(17): Xaa is Gln or Lys
  MOD_RES (20)..(20): Aib
  MISC_FEATURE (21)..(21): Xaa is Glu or Lys
  MISC_FEATURE (24)..(24): Xaa is Asn or Gln
  MISC_FEATURE (28)..(28): Xaa is Ala or Glu
  MOD_RES (35)..(35): AMIDATION
YXEGTFTSDYSIYLXXXAAXXFVXWLLXGGPSSGA

SEQ ID NO: 17 - protein, 4 aa, Artificial Sequence
  Synthetic
SSGA

SEQ ID NO: 18 - protein, 6 aa, Artificial Sequence
  Synthetic
  MOD_RES (5)..(5): Aib
KQAAXE

SEQ ID NO: 19 - protein, 6 aa, Artificial Sequence
  Synthetic
  MOD_RES (5)..(5): Aib
KKAAXE

SEQ ID NO: 20 - protein, 6 aa, Artificial Sequence
  Synthetic
  MOD_RES (5)..(5): Aib
KQAAXK

SEQ ID NO: 21 - protein, 6 aa, Artificial Sequence
  Synthetic
  MOD_RES (5)..(5): Aib
EQAAXK

(19) United States
(12) Patent Application Publication
(10) Pub. No.: US 2023/0346961 A1
(43) Pub. Date: Nov. 2, 2023

(54) GLP-1 AND GIP RECEPTOR CO-AGONISTS

(71) Applicant: Novo Nordisk A/S, Bagsvaerd (DK)

(72) Inventors: Patrick J. Knerr, Plainfield, IN (US); Brian Finan, Indianapolis, IN (US); Fa Liu, Watchung, NJ (US); Richard DiMarchi, Carmel, IN (US)

(21) Appl. No.: 18/016,947

(22) PCT Filed: Jul. 22, 2021
(86) PCT No.: PCT/EP2021/070483

§ 371 Date: Jan. 19, 2023

Related U.S. Application Data
(60) Provisional application No. 63/055,063, filed on Jul. 22, 2020.

(30) Foreign Application Priority Data
Aug. 24, 2020 (EP) 20192415.6

(51) Int. Cl. A61K 47/64; C07K 14/605; A61P 3/04; A61P 3/10

(52) U.S. Cl. CPC A61K 47/64; C07K 14/605; A61P 3/04; A61P 3/10

(57) ABSTRACT

Peptide co-agonists of the human GLP-1 and GIP receptors, long-acting derivatives thereof and their medical use in treatment and/or prevention of obesity, diabetes, and/or liver diseases are described.

### TECHNICAL FIELD

The present invention relates to compounds that are agonists of the glucagon-like peptide 1 (GLP-1) receptor and the glucose-dependent insulinotropic polypeptide (GIP) receptor with a protracted profile of action.

### INCORPORATION-BY-REFERENCE OF THE SEQUENCE LISTING

The present application is filed with a Sequence Listing in electronic form. The entire contents of the sequence listing are hereby incorporated by reference.

### BACKGROUND

Glucagon-like peptide 1 (GLP-1) is a gut enteroendocrine cell-derived hormone and one of two prominent endogenous physiological incretins. GLP-1 improves glycemic control by stimulating glucose-dependent insulin secretion in response to nutrients (glucose), inhibits glucagon secretion from the pancreatic alpha-cells, slows gastric emptying, and induces body weight loss primary by decreasing food consumption. Glucose-dependent insulinotropic polypeptide (GIP), the other prominent incretin, improves glycemic control by stimulation of insulin secretion in response to nutrients (fat, glucose). Furthermore, GIP appears to improve plasma lipid profile and to stimulate calcium accumulation in bones. In contrast to GLP-1, the incretin effect of GIP is severely reduced in type 2 diabetes patients, though recent studies suggest that GIP efficiency can be regained in these patients after treatment to improve glucose control. Nonetheless, the role of GIP to regulate systemic metabolism beyond its direct effect at the endocrine pancreas remains controversial, particularly as it relates to GIP action to promote gain in fat mass in animal models. These results have fostered beliefs that GIPR antagonism can improve body weight. Thus, employment of compounds acting at GIP receptors, and specifically whether to agonize or antagonize, as a strategy to improve body weight remains a contentious subject of intense scientific investigation (Finan et al, TRENDS Mol Med, 2016, 22 (5): 359-376; Killion et al, Endo Rev, 2020, 41 (1): 1-21).

Protracted GIP analogues have been shown to lower body weight and improve glycemic control, though comparatively less potent than GLP-1 analogues to lower body weight in rodent models (Mroz et al, Mol Metab, 2019, 20: 51-62). Moreover, GIP analogues induce body weight loss by additive/synergistic action with GLP-1 analogues in dual administration (Finan et al, Sci Transl Med, 2013, 5 (209): 209ra151; Nørregaard et al, Diabetes Obes Metab, 2018, 20 (1): 60-68), and as such represent suitable candidates for amplification of GLP-1-based pharmacology. GIPR agonism can be recruited as a non-redundant partner to GLP-1R agonism as a single molecule co-agonist to amplify GLP-1 metabolic benefits, as has been shown in preclinical animal models, most notably body weight loss and glycemic control (Finan et al, Sci Transl Med, 2013, 5 (209): 209ra151; Coskun et al, Mol Metab, 2018, 18: 3-14). Two different peptides with high potency dual incretin receptor agonism have advanced to multi-dose clinical studies. The clinical results have demonstrated improvements in glycemic control and body weight that exceeds what is achieved with comparable dosing of benchmark GLP-1 specific agonists (Frias et al, Cell Metab, 2017, 26 (2): 343-352; Frias et al, Lancet, 2018, 392 (10160): 2180-2193), demonstrating the translational aspects and therapeutic benefits of co-targeting GLP-1 and GIP receptors.

GLP-1/GIP receptor co-agonists and their potential medical uses are described in several patent applications such as WO 2010/011439, WO 2013/164483, WO 2014/192284, WO 2015/067715, WO 2015/022420, WO 2015/086728, WO 2015/086729, WO 2016/111971, WO 2020/023386, U.S. Pat. No. 9,745,360, US 2014/162945, and US 2014/0357552. However, no co-agonistic products have so far obtained market approval.

### SUMMARY

The present invention relates to single molecule co-agonists comprising a peptide and a substituent, which react with both the human GLP-1 and GIP receptors with high potency and display a protracted profile suitable for once weekly dosing regime in humans. This is achieved by the combination of certain peptide sequence variants with substituents via a single site acylation with a diacid based fatty acid.

An aspect of the invention relates to a peptide having the amino acid sequence

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉

with an optional amide modification of the C-terminus; wherein
X₂ is Aib
X₁₅ is D or E
X₁₆ is E or K
X₁₇ is Q or K
X₂₀ is Aib
X₂₁ is E or K
X₂₄ is N or Q
X₂₈ is A or E
X₃₂ is S or absent
X₃₃ is S or absent
X₃₄ is G or absent
X₃₅ is A or absent
X₃₆ is P or absent
X₃₇ is P or absent
X₃₈ is P or absent
X₃₉ is S or absent.

An aspect of the invention relates to a compound comprising a peptide and a substituent; wherein the amino acid sequence of the peptide is:

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉,

with an optional amide modification of the C-terminal amino acid residue; wherein
X₂ is Aib
X₁₅ is D or E
X₁₆ is E or K
X₁₇ is Q, R or K
X₂₀ is Aib
X₂₁ is E or K
X₂₄ is N or Q
X₂₈ is A or E
X₃₂ is S or absent
X₃₃ is S or absent
X₃₄ is G or absent
X₃₅ is A or absent
X₃₆ is P or absent
X₃₇ is P or absent
X₃₈ is P or absent
X₃₉ is S or absent;
and a substituent attached via the epsilon-amino group of a Lysine (K) residue in position 16, 17 or 21;
or a pharmaceutically acceptable salt hereof.

A further aspect of the invention relates to a method for preparing the GLP-1/GIP receptor co-agonists described herein.

In a further aspect the invention relates to a pharmaceutical composition comprising the GLP-1/GIP receptor co-agonists compounds described herein.

A further aspect of the invention relates to medical use of the GLP-1/GIP receptor co-agonists described herein.

In one aspect the invention relates to use of the GLP-1/GIP receptor co-agonists described herein for prevention or treatment of diabetes, obesity, and/or liver diseases.

### BRIEF DESCRIPTION OF DRAWINGS

FIG. 1 shows the effect on body weight (expressed as percent change from starting body weight) in DIO mice treated with once-daily subcutaneous injections of vehicle or 3 nmol/kg of GLP-1/GIP receptor co-agonists 9, 17, 19, 20, 21, 22, 25 and 34.

### DESCRIPTION

In what follows, Greek letters may be represented by their symbol or the corresponding written name, for example: α=alpha; β=beta; ε=epsilon; γ=gamma; ω=omega; etc. Also, the Greek letter of may be represented by “u”, e.g. in μl=ul, or in μM=uM.

### GLP-1/GIP Receptor Co-Agonists

The present invention relates to compounds that are GLP-1 receptor and the GIP receptor agonists, also referred to as GLP-1/GIP receptor co-agonists or simply co-agonists.

The term “compound” is used herein to refer to a molecular entity, and “compounds” may thus have different structural elements besides the minimum element defined for each compound or group of compounds. It follows that a compound may be a peptide or a derivative thereof, as long as the compound comprises the defined structural and/or functional elements.

The term “compound” is also meant to cover pharmaceutically relevant forms hereof, i.e. a compound as defined herein or a pharmaceutically acceptable salt or ester thereof.

The term “analogue” generally refers to a peptide, the sequence of which has one or more amino acid changes when compared to a reference amino acid sequence. An “analogue” may also include amino acid elongations in the N-terminal and/or C-terminal positions and/or truncations in the N-terminal and/or C-terminal positions.

In general, amino acid residues may be identified by their full name, their one-letter code, and/or their three-letter code. These three ways are fully equivalent.

Amino acids are molecules containing an amino group and a carboxylic acid group, and, optionally, one or more additional groups, often referred to as a side chain.

The term “amino acid” includes proteinogenic (or natural) amino acids (amongst those the 20 standard amino acids), as well as non-proteinogenic (or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification). Non-limiting examples of non-proteinogenic amino acids are Aib (α-aminoisobutyric acid, or 2-aminoisobutyric acid), norleucine, norvaline as well as the D-isomers of the proteinogenic amino acids.

In what follows, each amino acid of the peptides for which the optical isomer is not stated is to be understood to mean the L-isomer (unless otherwise specified).

The GLP-1/GIP receptor co-agonists described herein comprise or consist of a peptide and a substituent. In some embodiments, the peptide is a synthetic peptide created to optimize the activity via the GLP-1 and GIP receptors. Compounds having a suitable receptor binding activity towards both the GLP-1 receptor and the GIP receptor have been identified as demonstrated in the examples herein.

The compounds further display an extended half-life gained by the substituent comprising a fatty acid group. The compound identified are thus considered attractive molecules suitable for further development.

In some embodiments, the carboxy terminus of a peptide holds a —COOH group. In some embodiments, the compounds may optionally include an amide group (C(═O)—NH₂) at the C-terminus, which is a naturally occurring modification substituting —OH with —NH₂, such as seen with native Exendin-4.

### Peptide

The GLP-1/GIP receptor co-agonists described herein comprise a peptide and a substituent as described below, in which the substituent is attached to the peptide backbone via an amino acid residue.

In some embodiments, the amino acid sequence of the peptide is

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉

with an optional amide modification of the C-terminus wherein;
X₂ is Aib
X₁₅ is D or E
X₁, is E or K
X₁₇ is Q or K
X₂₀ is Aib
X₂₁ is E or K
X₂₄ is N or Q
X₂₈ is A or E
X₃₂ is S or absent
X₃₃ is S or absent
X₃₄ is G or absent
X₃₅ is A or absent
X₃₆ is P or absent
X₃₇ is P or absent
X₃₈ is P or absent
X₃₉ is S or absent.

In one embodiment, X₃₉ are absent. In one embodiment, X₃₈ and X₃₉ are absent. In one embodiment, X₃₇, X₃₈ and X₃₉ are absent. In one embodiment, X₃, X₃₇, X₃₈ and X₃₉ are absent. In further such embodiments, X₃₂X₃₃X₃₄X₃₅ is SSGA.

In a further embodiment thereof, the peptide has an amide modification of the C-terminus.

In one embodiment, the peptide is

(SEQ ID NO.: 16)
YX₂EGTFTSDYSIYLX₁₅X₁₆X₁₇AAX₂₀X₂₁FVX₂₄WLLX₂₈GGPSSGA

wherein
X₂ is Aib
X₁₅ is D or E
X₁₆ is E or K
X₁₇ is Q or K
X₂₀ is Aib
X₂₁ is E or K
X₂₄ is N or Q
X₂₈ is A or E.

In one embodiment, X₁₆ is K. In one embodiment, X₁₆ is E. In one embodiment, X₁₇ is Q. In one embodiment, X₁₇ is K. In one embodiment, X₂₁ is E. In one embodiment, X₂₁ is K. In one embodiment, X₂₄ is N. In one embodiment, X₂₄ is Q. In one embodiment, X₂₈ is A. In one embodiment, X₂₈ is E.

In one embodiment, X₁₆X₁₇AAX₂₀X₂₁ is selected from the group consisting of: KQAAAibE, KKAAAibE, KQAAAibK and EQAAAibK. In one embodiment, X₁₆X₁₇AAX₂₀X₂₁ is KQAAAibE. In one embodiment, X₁₆X₁₇AAX₂₀X₂₁ is KKAAAibE. In one embodiment, X₁₆X₁₇AAX₂₀X₂₁ is KQAAAibK. In one embodiment, X₁₆X₁₇AAX₂₀X₂₁ is EQAAAibK.

In a further embodiment, the amino acid sequence of the peptide is any one of SEQ ID NO.: 2, 3, 7, 8, 9, 10, 11, 12, 13 and 14. In one embodiment the amino acid sequence of the peptide is any one of SEQ ID NO.: 7, 8, 9, 10, 11, 12, 13 and 14.

In one embodiment, the amino acid sequence of the peptide is SEQ ID NO.: 9.

In one embodiment, the amino acid sequence of the peptide is SEQ ID NO.: 10 or 13

In one embodiment, the amino acid sequence of the peptide is SEQ ID NO.: 10.

In one embodiment, the amino acid sequence of the peptide is SEQ ID NO.: 11 or 14 In one embodiment, the amino acid sequence of the peptide is any one of SEQ ID NO.: 7, 8, 9 and 12.

In further such embodiments, the peptide has an amide modification of the C-terminus.

### Derivatives

In some embodiments, the GLP-1 and GIP receptor agonists comprise or consist of a substituent as described below covalently linked to a peptide.

Such compounds may be referred to as derivatives of the peptide, as they are obtained by covalently linking a substituent to a peptide backbone.

An aspect of the invention relates to a compound comprising a peptide and a substituent; wherein the amino acid sequence of the peptide is:

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉

with an optional amide modification of the C-terminus, wherein;
X₂ is Aib
X₁₅ is D or E
X₁, is E or K
X₁₇ is Q or K
X₂₀ is Aib
X₂₁ is E or K
X₂₄ is N or Q
X₂₈ is A or E
X₃₂ is S or absent
X₃₃ is S or absent
X₃₄ is G or absent
X₃₅ is A or absent
X₃₆ is P or absent
X₃₇ is P or absent
X₃₈ is P or absent
X₃₉ is S or absent;
wherein the substituent is attached to the peptide via a Lysine (K) residue in position 16, 17 or 21;
or a pharmaceutically acceptable salt hereof.

In further embodiments, the peptide may be defined as described herein above.

### Substituent

In one embodiment, the substituents as described herein are attached to the peptides described herein via a lysine (K) residue in position 16, 17 or 21.

In one embodiment, the substituent is attached to the peptide via the epsilon-amino group of a Lysine (K) when said Lysine is included at position 16, 17 or 21.

In one embodiment, the substituent is a chemical structure covalently attached to the peptide that is capable of forming non-covalent complexes with plasma albumin, thereby promoting the circulation of the co-agonist with the blood stream, and also having the effect of protracting the time of action of the co-agonist, due to the fact that the complex of the co-agonist and albumin is only slowly removed by renal clearance.

In one embodiment, the substituent comprises a fatty acid group. In such an embodiment, the fatty acid group comprises a carbon chain which contains at least 8 consecutive —CH₂— groups. In one embodiment, the fatty acid group comprises at least 10 consecutive —CH₂— groups, such as least 12 consecutive —CH₂— groups, at least 14 consecutive —CH₂— groups, at least 16 consecutive —CH₂— groups, or such as at least 18 consecutive —CH₂— groups.

In one embodiment, the fatty acid group comprises 8-20 consecutive —CH₂— groups. In one embodiment, the fatty acid group comprises 10-18 consecutive —CH₂— groups. In one embodiment, the fatty acid group comprises 12-18 consecutive —CH₂— groups. In one embodiment, the fatty acid group comprises 14-18 consecutive —CH₂— groups.

In some embodiments, the substituent consists of several elements, such as a protractor element and one or more linker elements. In one embodiment, the term “protractor” is used to describe the fatty acid group which is the terminal part of the substituent responsible for extending half-life of the compound.

In one embodiment, the protractor (Prot) may be defined by:

Chem. 1: HOOC—(CH₂)ₙ—CO—* wherein n is an integer in the range of 8-20, which may also be referred to as a C(n+2) diacid or as

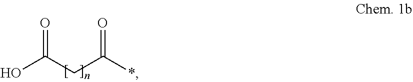

wherein n is an integer in the range of 8-20.

In one embodiment, the substituent further comprises one or more linker elements. In some embodiments, the linker elements are linked to each other and the protractor by amide bonds and referred to as “Z” (see further below).

As further defined herein below the number of linker elements may be at most 4, referred to as -Z1-Z2-Z3-Z4- where Z1 is connected with the protractor (Prot-) and the last Z element is connected with the peptide, in which case the substituent may be referred to as Prot-Z1-Z2-Z3-Z4-. The symbol * above thus indicates the attachment point to Z1, which when bound via an amide bond is a nitrogen. In an embodiment, where Z1 is a bond (see below), the symbol * indicates the attachment point to the nitrogen of the neighbouring Z element.

In one embodiment, the substituent is defined by: Prot-Z1-Z2-Z3-Z4- wherein Prot- is selected from Chem1, Chem 1b, and wherein n is an integer in the range of 16-20.

In a particular embodiment, n is 14, 15, 16, 17, 18, 19 or 20 in Chem. 1 or Chem. 1b.

In a particular embodiment, n is 14, 15, 16, 17, or 18 in Chem. 1 or Chem. 1b.

In a particular embodiment, n is 16 or 18 in Chem. 1 or Chem. 1b.

In a particular embodiment, n is 16, 17, 18, 19 or 20 in Chem. 1 or Chem. 1b.

In a particular embodiment, n is 16, 18 or 20 in Chem. 1 or Chem. 1b.

In a particular embodiment, n is 18 or 20 in Chem. 1 or Chem. 1b.

In a particular embodiment, the protractor (Prot) is a C18 diacid or a C20 diacid.

The term “bond” as used here means a covalent bond. When a linker element of Z1-Z4 is defined as a bond, it is equivalent to a situation wherein said linker element is absent.

The indication herein below that any of Z1-Z4 is a bond may also be read as any of Z1-Z4 being absent, so that the previous Z element is covalently linked to the next Z element that is not “a bond” (or absent).

In some embodiments, the linker elements Z1-Z4 are individually selected from chemical moieties capable of forming amide bonds, including amino acid like moieties, such as Glu, γGlu (also termed gamma Glu or gGlu and defined by *—NH—CH—(COOH)—CH₂—CH₂—CO—*), ε-Lys (also termed epsilon Lys or eLys and defined by *—NH—(CH₂)₄—CH(NH₂)—CO—*), Ser, Ala, Thr, Ado, Aeep and Aeeep and further moieties as described below.

In one embodiment, the Z1 element is optional. In one such embodiment, Z1 is selected from

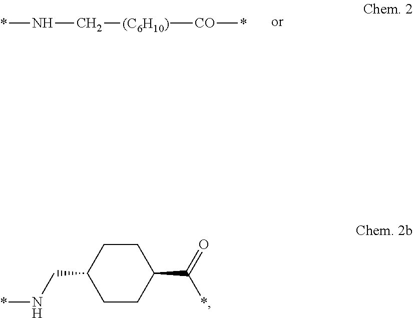

and a bond.

Chem. 2 may also be referred to as Trx for Tranexamic acid or trans-4-(aminomethyl)cyclohexanecarboxylic acid, where Chem 2. covers the (1,2), (1,3) and (1,4) forms, while Chem 2b specifies the (1,4) form.

In one embodiment, Z1 is Trx or a bond.

In one embodiment, Z2 is selected from γGlu, Glu, or a bond.

In one embodiment, Z2 is γGlu.

In one embodiment, Z3 and Z4, are selected, independently of each other, from Glu, ε-Lys, γGlu, Gly, Ser, Ala, Thr, Ado, Aeep, Aeeep and a bond.

Glu, Gly, Ser, Ala, Thr are amino acid residues well known in the art.

ε-Lys is defined by Chem. 3: *—NH—(CH₂)₄—CH(NH₂)—CO—*, which may also be described by

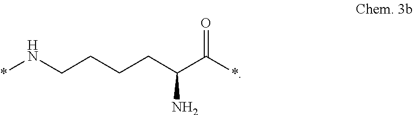

γGlu is defined by Chem. 4: *—NH—CH(COOH)—(CH₂)₂—CO—* which may also be described by

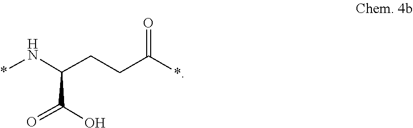

Ado is defined by Chem. 5: *—NH—(CH₂)₂—O—(CH₂)₂O—CH₂—CO—* may also be referred to as 8-amino-3,6-dioxaoctanoic acid and which may also be described by

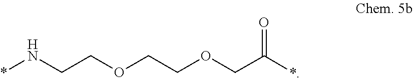

Aeep is defined by Chem. 6: *NH—CH₂CH₂OCH₂CH₂OCH₂CH₂CO*, which may also be described by

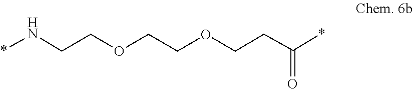 [SMILES: *NCCOCCOCCC(*)=O]

Aeeep is defined of Chem. 7: *NH—CH₂CH₂OCH₂CH₂OCH₂CH₂OCH₂CH₂CO*, which may also be described by

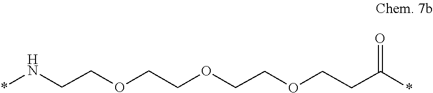 [SMILES: *NCCOCCOCCOCCC(*)=O]

In one embodiment, Z3 and Z4 are selected, independently of each other, from Glu, ε-Lys, γGlu, Gly, Ala, Ado, Aeep, Aeeep and a bond.

In one embodiment, Z3 and Z4 are selected, independently of each other, from Glu, ε-Lys, γGlu, Gly, Ala, Ado and a bond.

In one embodiment, Z3 and Z4 are selected, independently of each other, from Glu, ε-Lys, γGlu, Gly, Ado and a bond.

In one embodiment, Z3 and Z4 are selected, independently of each other, from ε-Lys, γGlu, Gly, Ado and a bond.

In one embodiment, Z3 and Z4 are selected, independently of each other, from ε-Lys, γGlu, Ado and a bond.

In one embodiment, Z3 and Z4 are ε-Lys or Ado.

In one embodiment, Z3 and Z4 are Ado.

In one embodiment, Z3 and Z4 are ε-Lys.

In one embodiment, the substituent is selected from substituents A, B, C, D, E, F and G defined as follows

| Substituent | | | | | |
|---|---|---|---|---|---|
| # | Prot | Z1 | Z2 | Z3 | Z4 |
| A | C18 diacid | — | γGlu | Ado | Ado |
| B | C18 diacid | — | γGlu | εLys | εLys |
| C | C20 diacid | — | γGlu | Ado | Ado |
| D | C20 diacid | — | γGlu | εLys | εLys |
| E | C20 diacid | Trx | γGlu | Ado | Ado |
| F | C20 diacid | Trx | γGlu | εLys | εLys |
| G | C18 diacid | — | γGlu | γGlu | γGlu |

In some embodiments, the substituent is covalently attached to a lysine residue of the co-agonist by acylation, i.e. via an amide bond formed between a carboxylic acid group of the substituent and the epsilon amino group of the lysine residue.

In one embodiment, the substituent is covalently attached to a lysine residue in position 16 of the peptide backbone by acylation, i.e., via an amide bond formed between a carboxylic acid group of the substituent and the epsilon amino group of the lysine residue.

In one embodiment, the substituent is covalently attached to a lysine residue in position 17 of the peptide backbone by acylation, i.e., via an amide bond formed between a carboxylic acid group of the substituent and the epsilon amino group of the lysine residue.

In one embodiment, the substituent is covalently attached to a lysine residue in position 21 of the peptide backbone by acylation, i.e., via an amide bond formed between a carboxylic acid group of the substituent and the epsilon amino group of the lysine residue.

The co-agonists may exist in different stereoisomeric forms having the same molecular formula and sequence of bonded atoms but differing only in the three-dimensional orientation of their atoms in space. The stereoisomerism of the exemplified co-agonists is indicated in the experimental section, in the names as well as the structures, using standard nomenclature. Unless otherwise stated the invention relates to all stereoisomeric forms of the embodied derivative.

### Functional Receptor Activation Activity

The functional activity of the GLP-1/GIP receptor agonists as described herein can be tested in vitro as described herein in Example 2.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed.

The in vitro potency of compounds may thus be determined as described herein and the EC₅₀ determined. The lower the EC₅₀ value, the better the potency.

In order to characterize such compounds, it may further be relevant to consider the in vitro potencies relative to the native hormones of each receptor.

The in vitro potency may, e.g., be determined in a medium containing membranes expressing the appropriate GLP-1 and/or GIP receptor, and/or in an assay with whole cells expressing the appropriate GLP-1 and/or GIP receptor.

For example, the functional response of the human or mouse GLP-1 and/or GIP receptor may be measured in a reporter gene assay, e.g. in a stably transfected BHK cell line that expresses the human or mouse GLP-1 and/or GIP receptor and contains the DNA for the cAMP response element (CRE) coupled to a promoter and the gene for firefly luciferase (CRE luciferase). When cAMP is produced as a result of activation of the GLP-1 and/or GIP receptor, this in turn results in luciferase being expressed. Luciferase may be determined by adding luciferin, which by the enzyme is converted to oxyluciferin and produces bioluminescence, which is measured as a reporter of the in vitro potency. One example of such an assay is described in Example 2 as described herein. Since the compounds may include a substituent designed to bind albumin, it is also important to note that the receptor activity may be affected by the presence or absence of human serum albumin (HSA) in the assay medium. A decrease in potency of the compound in the presence of HSA, indicated by an increase in EC₅₀ compared to the EC₅₀ in the absence of HSA, indicates interaction of the compounds with HSA and predicts a protracted time of action in vivo.

In one embodiment, the compounds have potent in vitro effects to activate the human GLP-1 and GIP receptors.

In one embodiment, the compounds are capable of activating the human GLP-1 and GIP receptors in vitro with an EC₅₀ of less than 50 pM, such as less than 40 pM, such as less than 30 pM, in CRE luciferase reporter assays as described in Example 2 herein, when performed without HSA.

In one embodiment, the compounds have an in vitro potency at the human GLP-1 and GIP receptors determined using the method of Example 2 corresponding to an EC₅₀ at or below 100 pM, such as below 50 pM, or such as below 20 pM.

In one embodiment, the EC₅₀ in human GLP-1 and GIP receptors assays are both 1-30, such as 1-25 pM, such as 1-20 pM, such as 1-15 pM or such as 1-10 pM.

In one embodiment, the compounds have potent in vitro effects to activate also the mouse GLP-1 and GIP receptors. In some embodiments, the compounds have an approximately equal in vitro potency between human and mouse GLP-1 receptors, and between human and mouse GIP receptors, when normalized to the respective native hormones of each receptor.

In a further particular embodiment, the derivatives have an in vitro potency at mouse GLP-1 and GIP receptors determined using the method of Example 2 corresponding to an EC₅₀ at or below 500 pM, more preferably below 200 pM, or most preferably below 100 pM.

In a further embodiment, the derivatives are capable of activating the human GLP-1 and GIP receptors selectively over the human glucagon receptor. The term “selectively” when used in relation to activation of the GLP-1 and GIP receptors over the glucagon receptor refers to derivatives that display at least 10 fold, such as at least 50 fold, at least 500 fold, or at least 1000 fold higher potency for the GLP-1 and GIP receptor compared to the glucagon receptor when measured in vitro. As described above, the potency assay for receptor function, such as an CRE luciferase functional potency assay, and the EC₅₀ values obtained compared.

### Pharmacokinetics Properties

The pharmacokinetic properties of the co-agonistic compounds may further be determined in vivo via pharmacokinetic (PK) studies. Animal models such as the mouse, rat, monkey, dog, or pig may be used to perform this characterization.

In such studies, animals are typically administered with a single dose of the drug, either intravenously, subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed. An important parameter is the terminal half-life as a long half-life indicates that less frequent administration of a compound may be possible. The terminal half-life (t½) in vivo after i.v. administration may be measured in minipigs described in Example 3.

In one embodiment, the terminal half-life is half-life (t½) in vivo in minipigs after iv. administration, e.g. as described in Example 3 herein.

In one embodiment, the terminal half-life in minipigs is at least 24 hours, such as at least 40 hours, or such as at least 60 hours.

### Biological Activity

The biological effects of co-agonistic compounds may further be studied in vivo using suitable animal models is known in the art, as well as in clinical trials.

The diet-induced obese (DIO) mouse is one example of a suitable animal model, and the effect on body weight, food intake, and glucose tolerance can be assessed during sub-chronic dosing in this model. The effect of the compounds of the invention on body weight, food intake, and glucose tolerance may be determined in such mice in vivo, e.g. as described in Example 4 herein.

In one embodiment, the compounds display the ability to reduce body weight, food intake, and improve glucose tolerance in DIO mice as described in Example 4.

In one embodiment, the compounds reduce body weight in DIO mice.

In one embodiment, the compounds reduce food intake in DIO mice.

In one embodiment, the compounds improve glucose tolerance in DIO mice.

In one embodiment, the compound reduces body weight by at least 20% after once daily administration of 3 nmol/kg of said compound for 10 days in DIO mice.

In one embodiment, the compound reduces food intake by at least 20% after once daily administration of 3 nmol/kg of said compound for 10 days in DIO mice. In one embodiment, the compounds improve glucose tolerance by at least 20% as measured in an IPGTT (intraperitoneal glucose tolerance test.

In one embodiment, the compound is selected from the group consisting of:

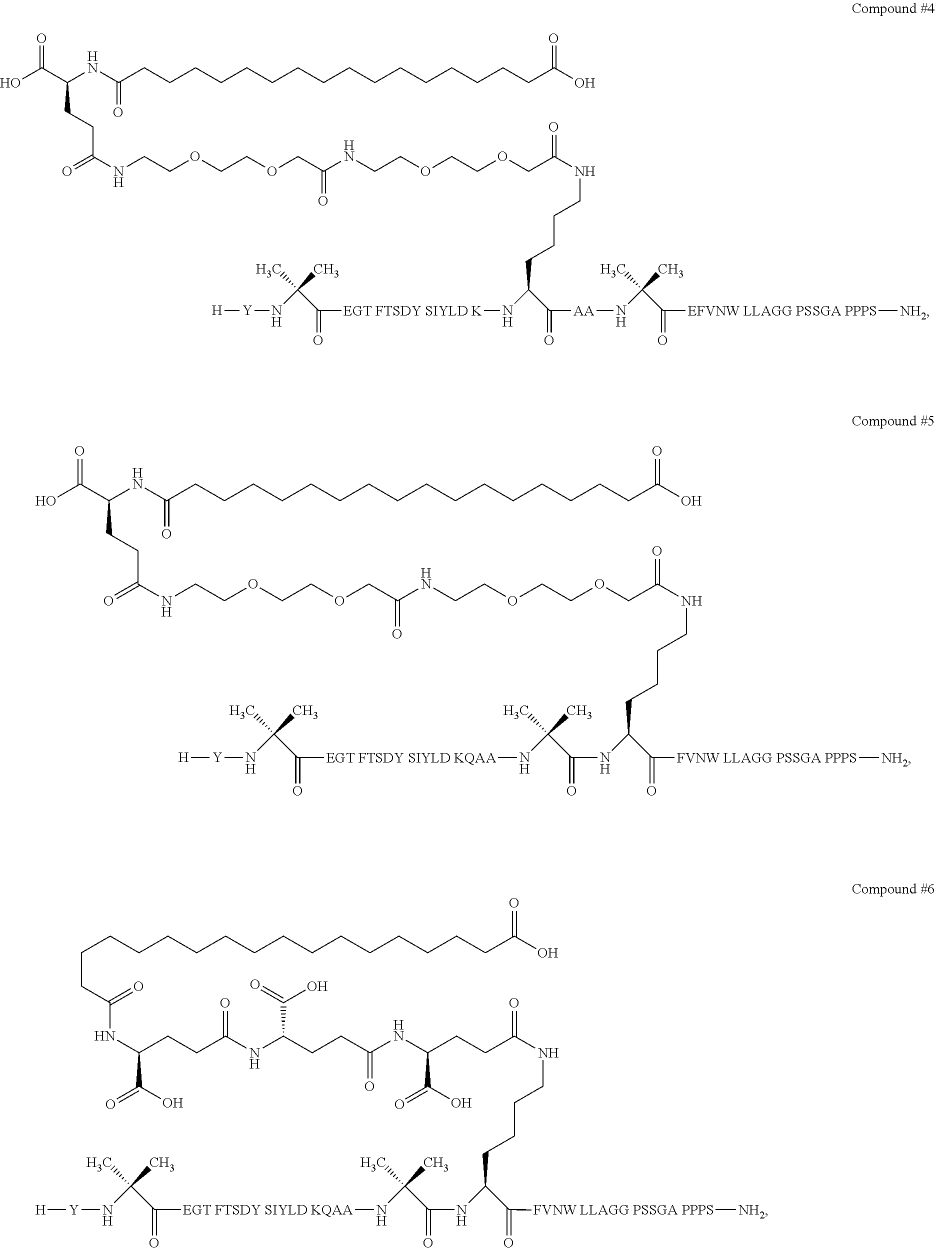

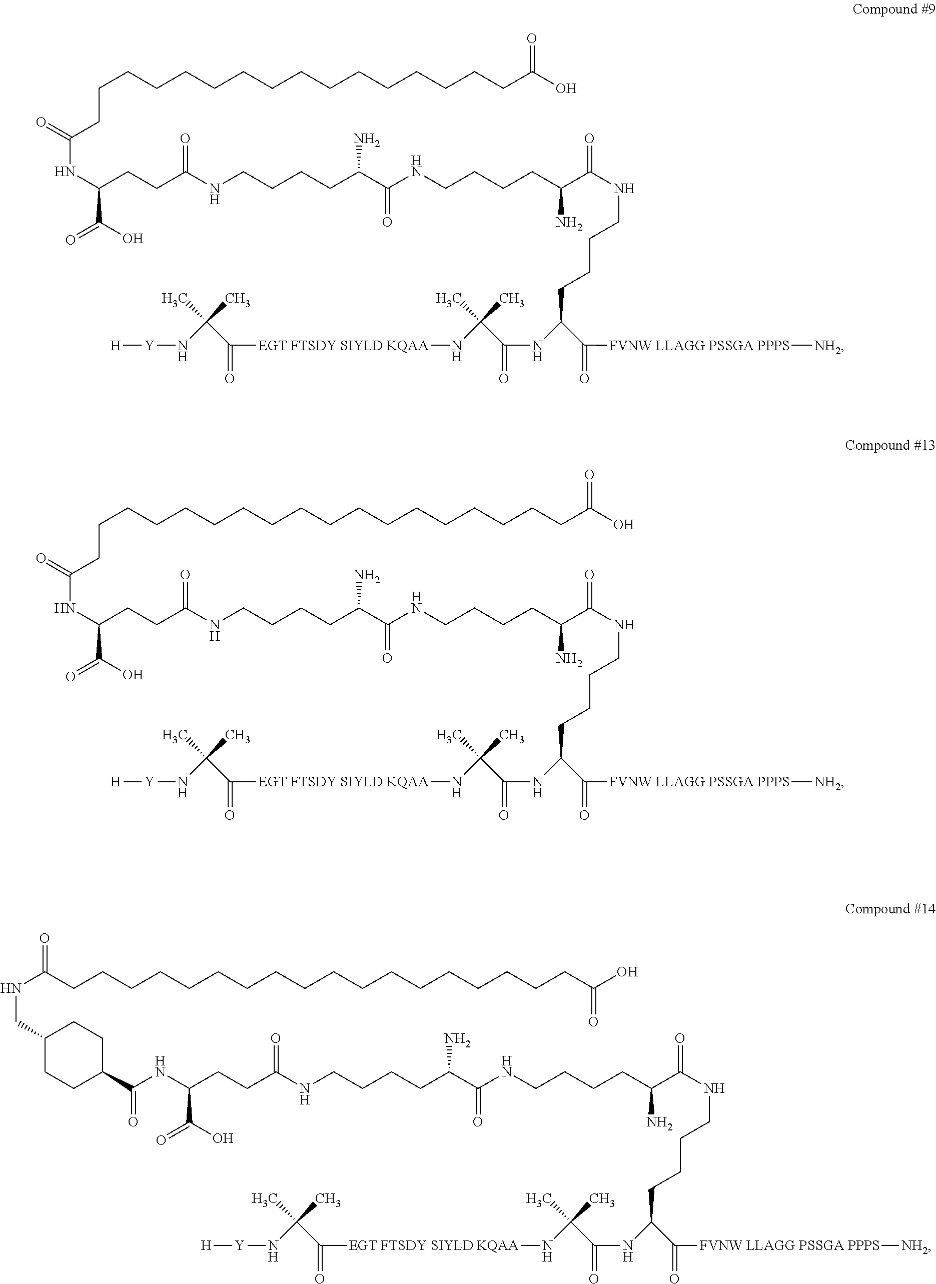

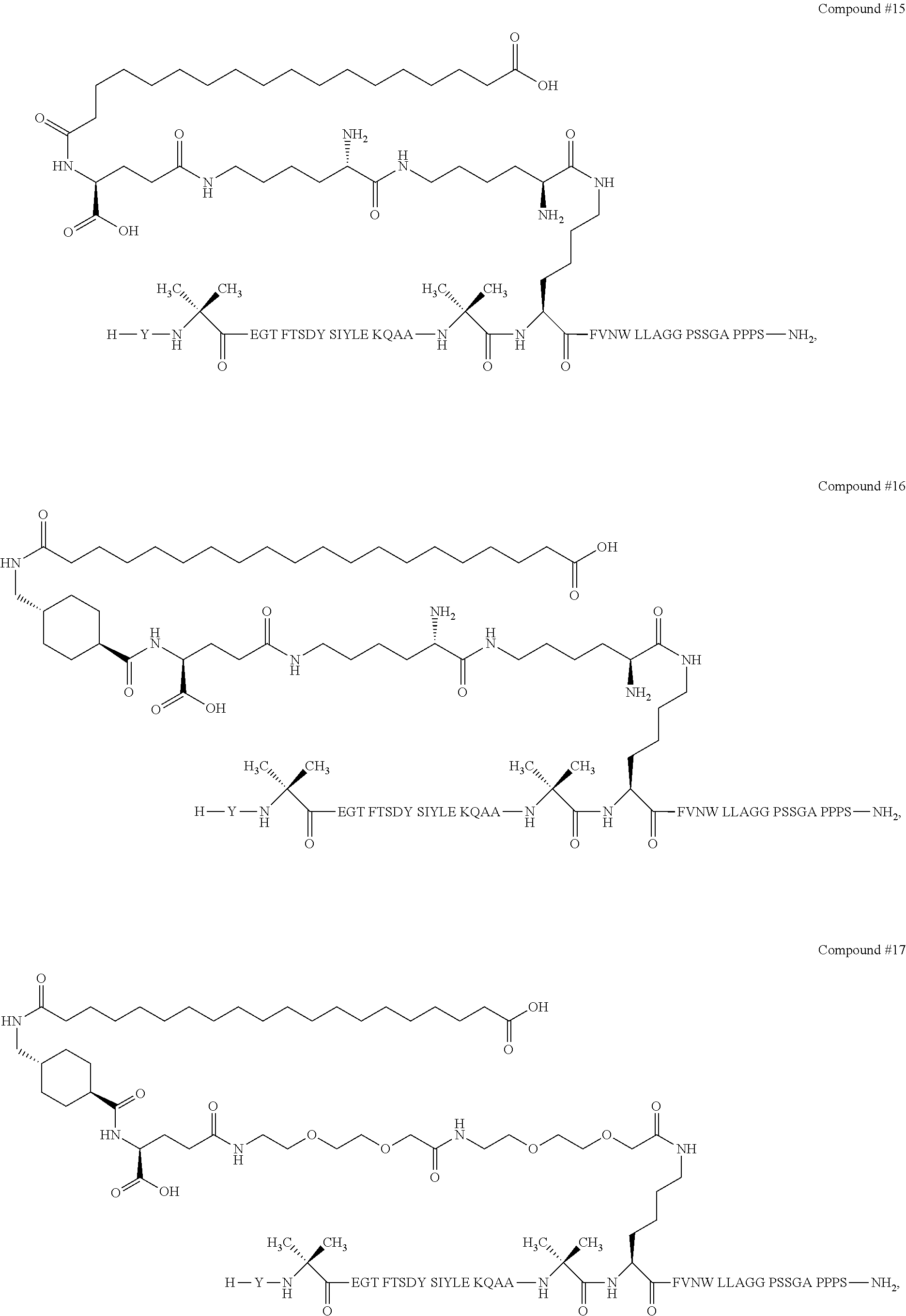

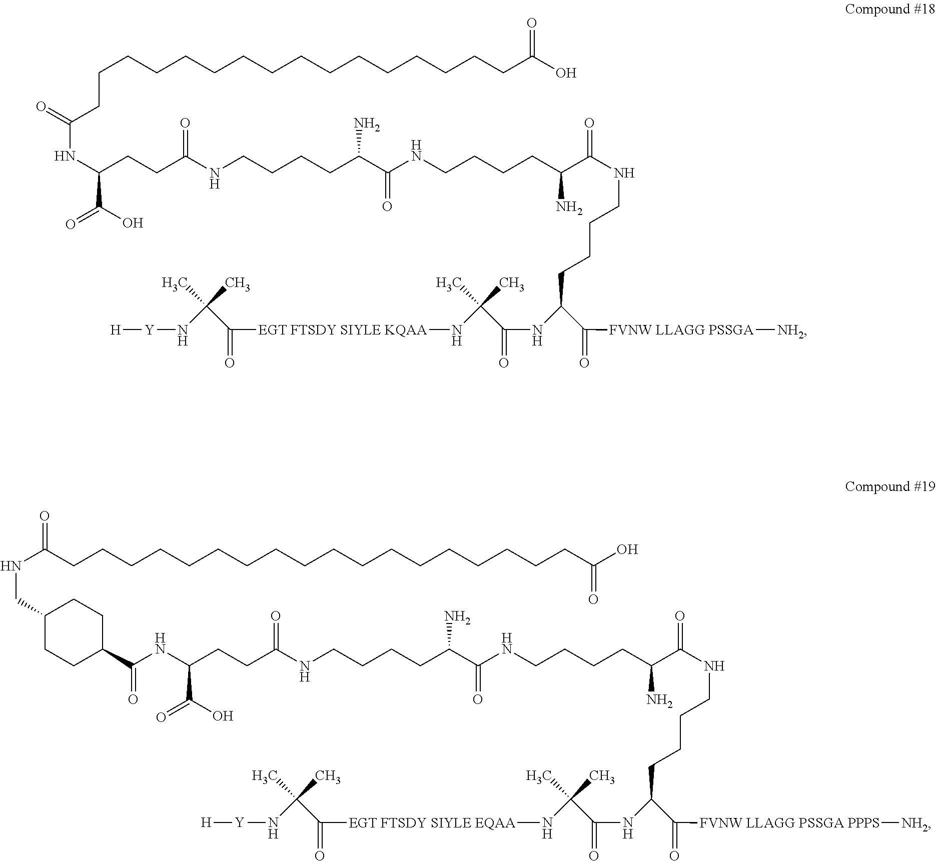

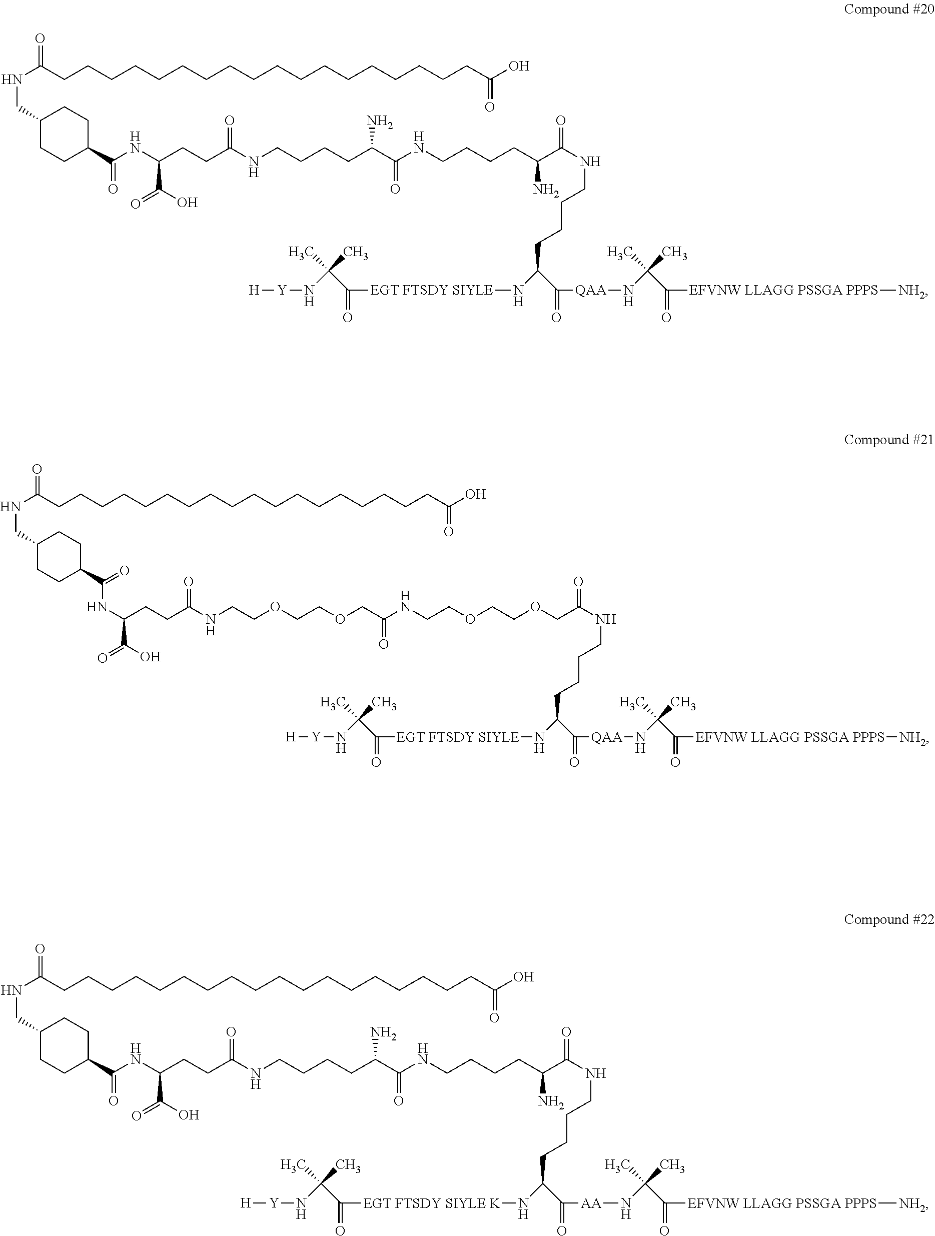

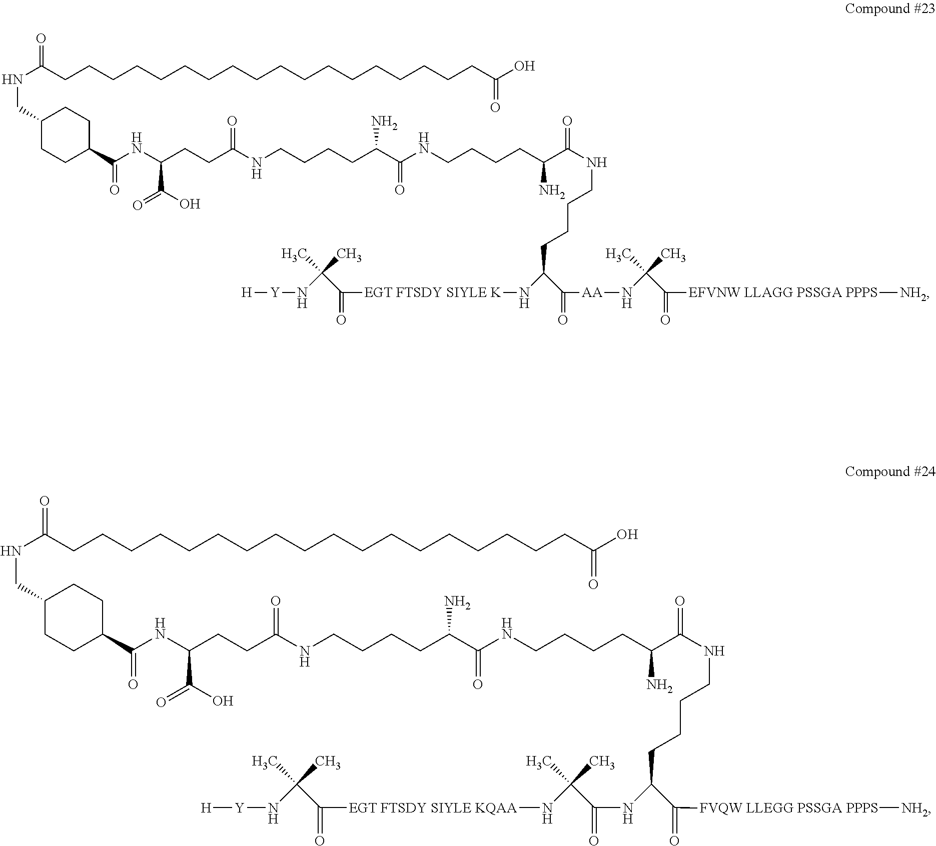

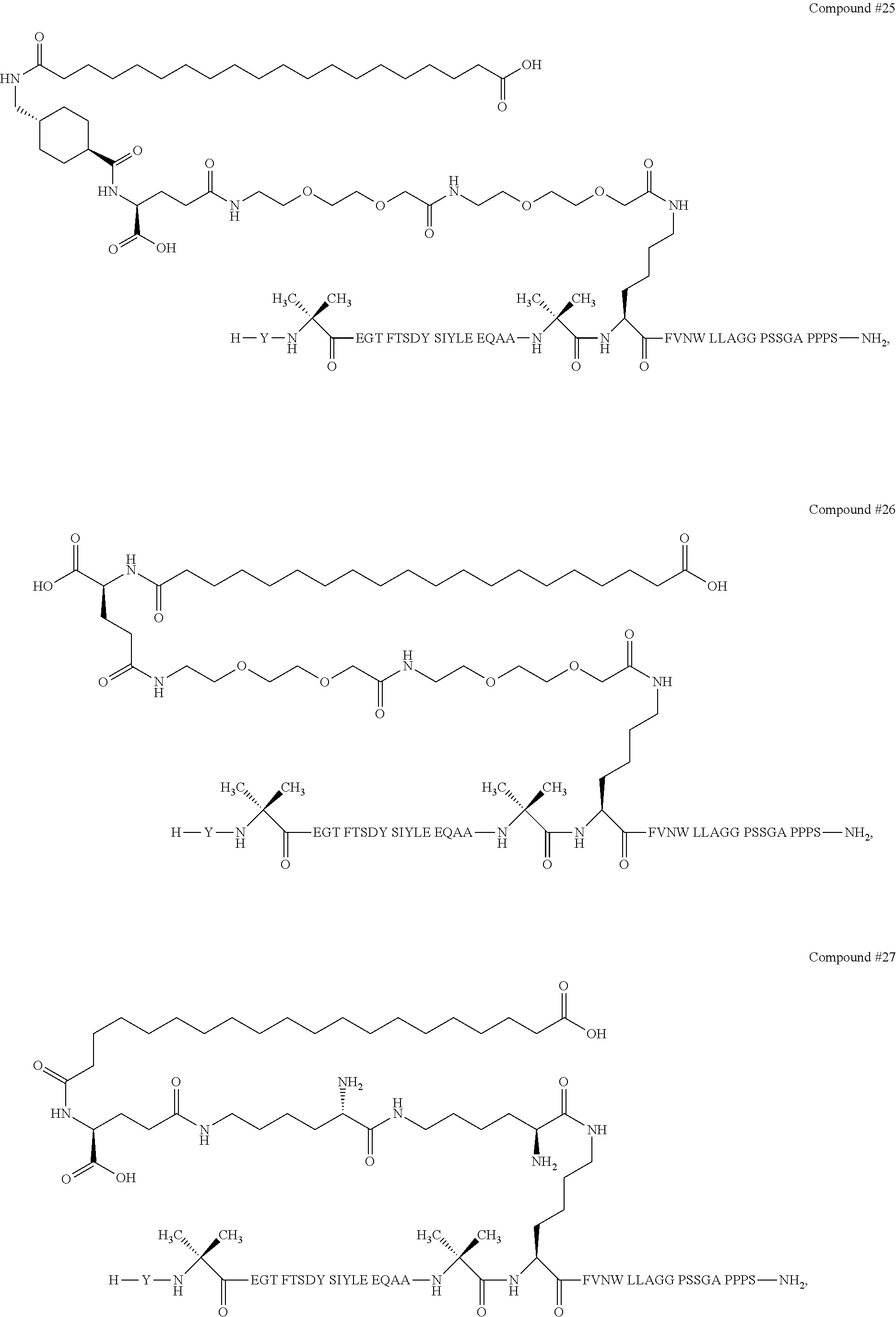

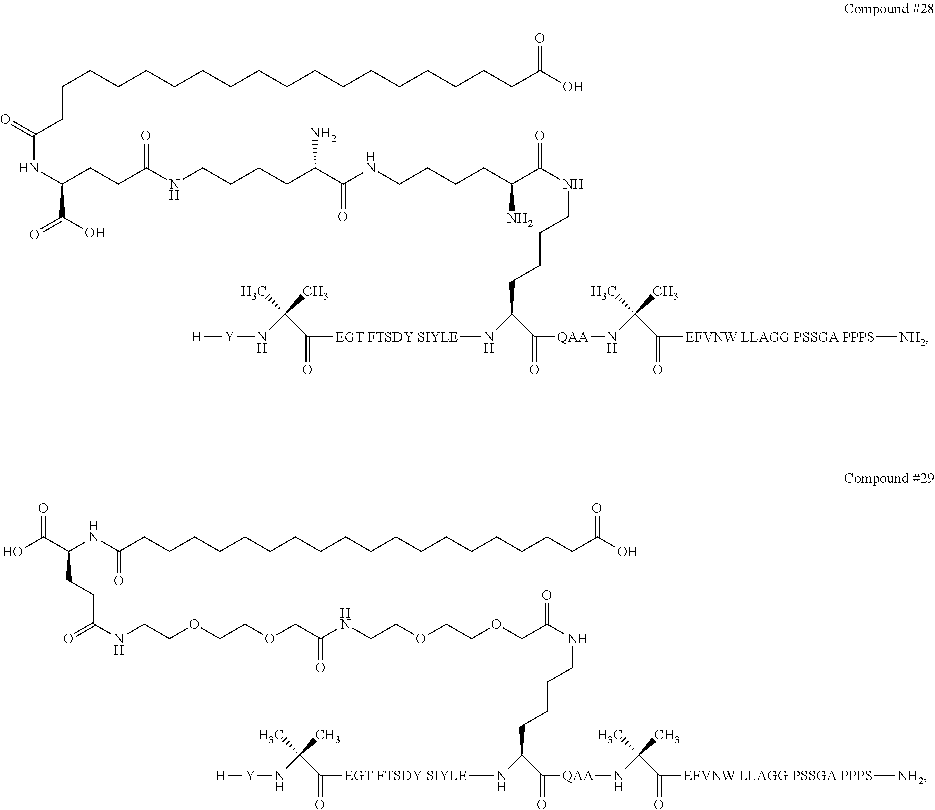

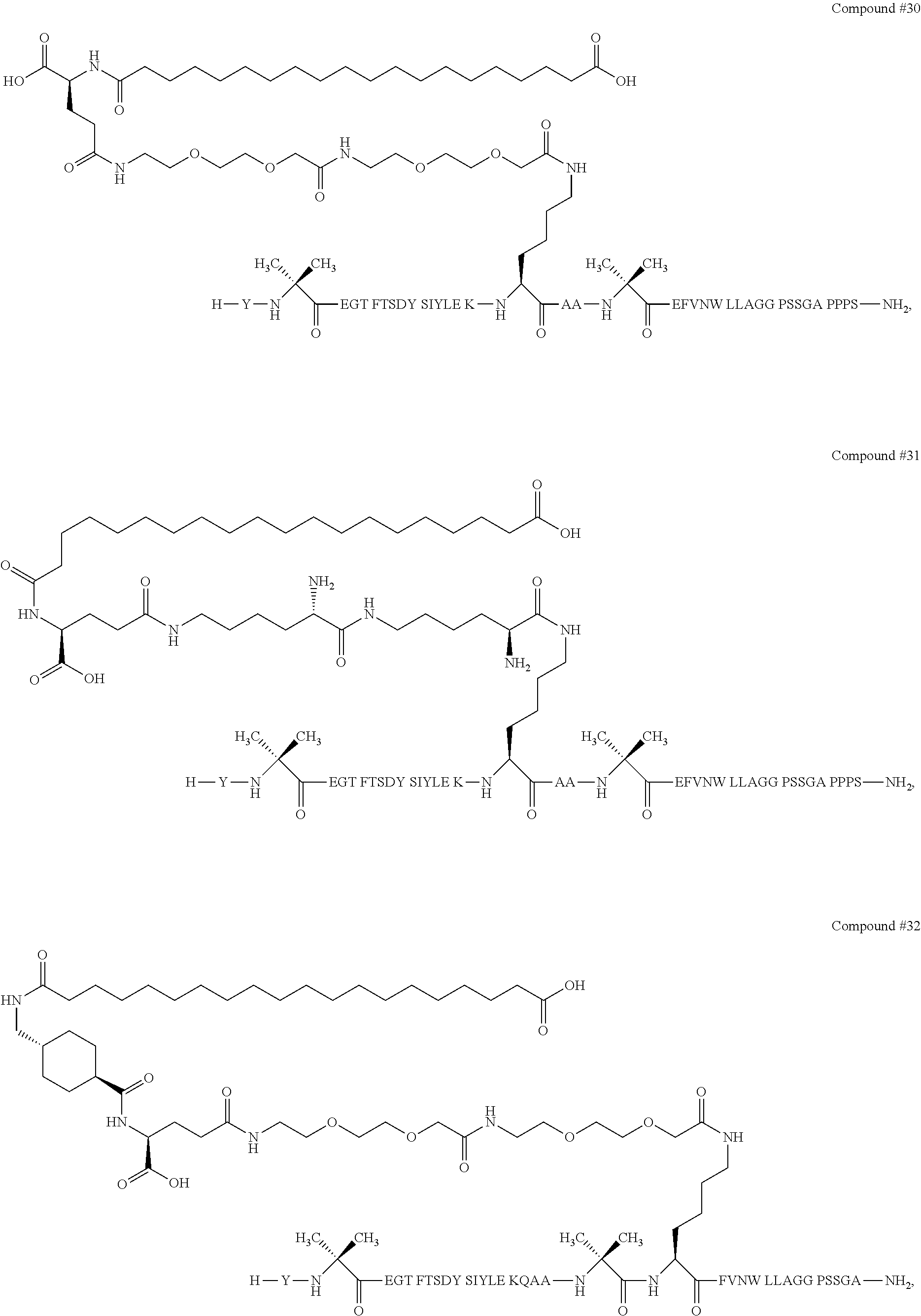

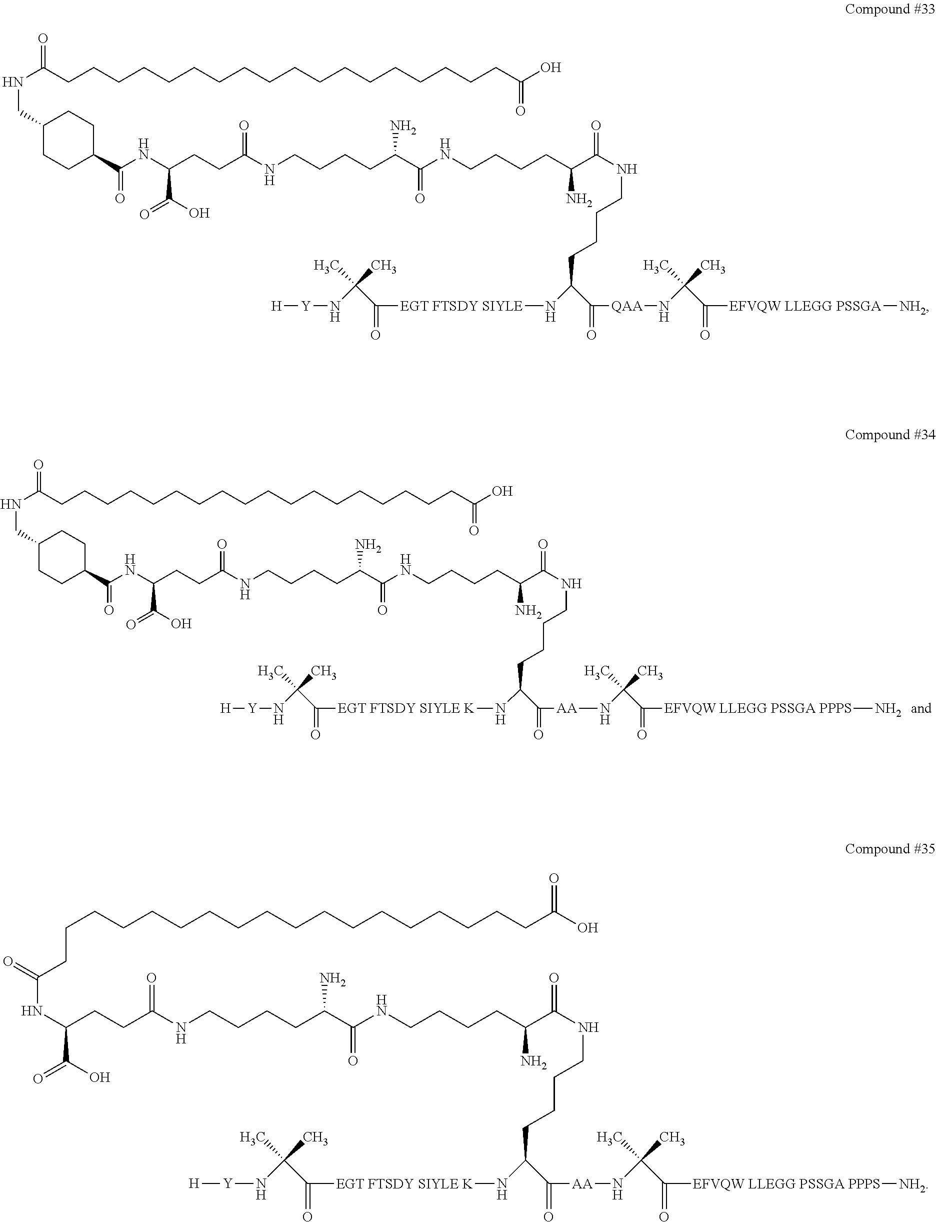

In one embodiment, the compound is selected from the group consisting of compounds #16, #17 and #19-35.

In one embodiment, the compound is selected from the group consisting of compounds #20, #21, #28, #29 and #33.

In one embodiment, the compound is selected from the group consisting of compounds #22, #23, #30, #31, #34 and #35.

In one embodiment, the compound is selected from the group consisting of compound #34 and compound #35.

In one embodiment, the compound is selected from the group consisting of compounds #16, #17, #19, #24, #25, #26, #27 and #32.

In one embodiment, the compound is selected from the group consisting of compounds #19, #25, #26 and #27.

### Pharmaceutically Acceptable Salts

In some embodiments, the co-agonists as described herein are in the form of a pharmaceutically acceptable salt. Salts are e.g. formed by a chemical reaction between a base and an acid, e.g.: 2NH₃+H₂SO₄→(NH₄)₂SO₄. The salt may be a basic salt, an acid salt, or it may be neither (i.e. a neutral salt). Basic salts produce hydroxide ions and acid salts hydronium ions in water. The salts of the compounds may be formed with added cations or anions between anionic or cationic groups, respectively. These groups may be situated in the peptide, and/or in the substituent of the compounds. Non-limiting examples of anionic groups include any free carboxylic acid groups in the substituent, if any, as well as in the peptide. The peptide moiety may include a free carboxylic acid group at the C-terminus, if present, as well as any free carboxylic acid group of internal acidic amino acid residues such as Asp and Glu.

Non-limiting examples of cationic groups include any free amino groups in the substituent, if any, as well as in the peptide. The peptide may include a free amino group at the N-terminus, if present, as well as any free imidazole or amino group of internal basic amino acid residues such as His, Arg, and Lys.

In a particular embodiment, the peptide or derivative is in the form of a pharmaceutically acceptable salt.

### Production Processes

The co-agonists may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999; Florencio Zaragoza Dörwald, “Organic Synthesis on Solid Phase”, Wiley-VCH Verlag GmbH, 2000; and “Fmoc Solid Phase Peptide Synthesis”, Edited by W. C. Chan and P. D. White, Oxford University Press, 2000.

Alternatively, the compounds may be produced by recombinant methods, e.g. by culturing a host cell containing a DNA sequence encoding the peptide sequence and capable of expressing the peptide, in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

The co-agonists that include non-natural amino acids and/or covalently attached substituents may be produced as described in the experimental part.

Specific examples of methods of preparing a number of co-agonists are included in the experimental part.

A further aspect of the invention relates to a method for preparing the peptides described herein.

A further aspect of the invention relates to a method for preparing the GLP-1/GIP receptor co-agonists described herein.

In one embodiment, the method for preparing a compound as described herein comprises a step of solid phase peptide synthesis. The substituent may be built sequentially as part of the solid phase peptide synthesis or produced separately and attached via the lysine residue after peptide synthesis.

In one embodiment, the compounds are produced by a two-step process whereby two peptide fragments are ligated after attachment of the substituent to one of the peptide fragments.

### Pharmaceutical Compositions

In a further aspect the invention relates to a pharmaceutical composition comprising a GLP-1/GIP receptor co-agonist as described herein. Compositions comprising the compound or a pharmaceutically acceptable salt hereof, and optionally one or more a pharmaceutically acceptable excipients may be prepared as is known in the art.

Liquid compositions, suitable for injection, can be prepared using conventional techniques of the pharmaceutical industry which involve dissolving and mixing the ingredients as appropriate to give the desired end product. Thus, according to one procedure, a GLP-1/GIP receptor co-agonist as described herein is dissolved in a suitable buffer at a suitable pH. The composition may be sterilized, for example, by sterile filtration.

The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, tablet aid, and/or to improve administration, and/or to improve absorption of the active substance.

The formulation of pharmaceutically active ingredients with various excipients is known in the art, see e.g. Remington: The Science and Practice of Pharmacy (e.g. 19th edition (1995), and any later editions).

In one embodiment, the pharmaceutical composition is a liquid formulation, such as an aqueous formulation.

### Pharmaceutical Indications

A further aspect of the invention relates to the use of GLP-1/GIP receptor co-agonist compounds as described herein as a medicament.

In one embodiment, the compounds described herein are for use in the following medical treatments:
(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(ii) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(iii) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(iv) weight maintenance after successful weight loss (either drug induced or by diet and exercise)—i.e. prevention of weight gain after successful weight loss.
(v) prevention and/or treatment of liver disorders, such as hepatic steatosis, non-alcoholic fatty liver disease (NAFLD), non-alcoholic steatohepatitis (NASH), liver inflammation or fatty liver;

In one embodiment, the compounds are for use in a method for prevention and/or treatment of diabetes and/or obesity.

In one embodiment, the compounds are for use in a method for treatment of diabetes and/or obesity.

In one embodiment, the compounds are for use in a method for treatment or prevention of type 2 diabetes.

In one embodiment, the compounds are for use in a method for treatment of type 2 diabetes.

In one embodiment, the compounds are for use in a method for treatment or prevention of obesity.

In one embodiment, the compounds are for use in a method for treatment of obesity.

In one embodiment, the compounds are for use in a method for weight management. In one embodiment, the compounds are for use in a method for reduction of appetite. In one embodiment, the compounds are for use in a method for reduction of food intake.

### EMBODIMENTS

1. A compound comprising a peptide and a substituent; wherein the amino acid sequence of the peptide is:

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉,

with an optional amide modification of the C-terminal amino acid residue; wherein
X₂ is Aib
X₁₅ is D or E
X₁, is E or K
X₁₇ is Q or K
X₂₀ is Aib
X₂₁ is E or K
X₂₄ is N or Q
X₂₈ is A or E
X₃₂ is S or absent
X₃₃ is S or absent
X₃₄ is G or absent
X₃₅ is A or absent
X₃₆ is P or absent
X₃₇ is P or absent
X₃₈ is P or absent
X₃₉ is S or absent;
and wherein the substituent is attached to the peptide via a Lysine (K) residue in position 16, 17 or 21;
or a pharmaceutically acceptable salt hereof.

2. The compound according to embodiment 1, wherein X₃₆, X₃₇, X₃₈ and X₃₉ are absent.

3. The compound according to embodiment 1 or 2, wherein X₃₂X₃₃X₃₄X₃₅ is SSGA.

4. The compound according to any one of embodiments 1-3, wherein the peptide has the amide modification of the C-terminus.

5. The compound according to embodiment 1, wherein the amino acid sequence of the peptide is

(SEQ ID NO.: 16)
YX₂EGTFTSDYSIYLX₁₅X₁₆X₁₇AAX₂₀X₂₁FVX₂₄WLLX₂₈GGPSSGA

wherein
   X₂ is Aib
   X₁₅ is D or E
   X₁₆ is E or K
   X₁₇ is Q or K
   X₂₀ is Aib
   X₂₁ is E or K
   X₂₄ is N or Q
   X₂₈ is A or E.

6. The compound according to any of the previous embodiments, wherein X₁₆X₁₇AAX₂₀X₂₁ is selected from the group consisting of: KQAAAibE, KKAAAibE, KQAAAibK and EQAAAibK.

7. The compound according to embodiment 1, wherein the amino acid sequence of the peptide is any one of SEQ ID NO.: 2, 3, 7, 8, 9, 10, 11, 12, 13 and 14.

8. The compound according to embodiment 0, wherein the peptide has the amide modification of the C-terminus.

9. The compound according to any of the previous embodiments, wherein the compound activates the human GLP-1 and GIP receptors in vitro with an EC₅₀ of less than 30 pM when measured without HSA in a CRE luciferase reporter assays as described in Example 2.

10. The compound according to any of the previous embodiments, wherein the compound has a half-life in mini-pigs of at least 60 hours.

11. The compound according to any of the previous embodiments, wherein the compound reduces bodyweight at least 20% in DIO mice by once daily administration of 3 nmol/kg over 10 days.

12. The compound according to any of the previous embodiments 1-11, wherein the substituent is attached via 16Lys.

13. The compound according to any of the previous embodiments 1-11, wherein the substituent is attached via 17Lys.

14. The compound according to any of the previous embodiments 1-11, wherein the substituent is attached via 21 Lys.

15. The compound according to any of the previous embodiments wherein the substituent is attached to the peptide via the epsilon-amino group of a Lysine (K).

16. The compound according to any of the previous embodiments, wherein the substituent comprises at least one protractor.

17. The compound according to embodiment 16, wherein the protractor is a fatty acid group.

18. The compound according to embodiment 16, wherein the protractor is a diacid, defined by Chem. 1: HOOC—(CH₂)ₙ—CO—, wherein n is an integer in the range of 12-20, such as n=16 or 18.

19. The compound according to any of the previous embodiments, wherein the substituent comprises at least one linker element.

20. The compound according to embodiment 19, wherein the substituent comprises at most four linker elements.

21. The compound according to embodiment 19, wherein the substituent comprises at most four linker elements referred to as -Z1-Z2-Z3-Z4-, where -Z1- is connected with the protractor and -Z4- is connected to the peptide.

22. The compound according to any of the embodiments 1-14, wherein the substituent is:
Prot-Z1-Z2-Z3-Z4-
wherein
Prot is C18 diacid or C20 diacid
Z1 is Trx or a bond
Z2 is γGlu, Glu, or a bond
Z3 is ε-Lys, γGlu, Gly or Ado and
Z4 is ε-Lys, γGlu, Gly or Ado.

23. The compound according to embodiment 22, wherein -Z1- is -Trx-.

24. The compound according to embodiment 22, wherein -Z2- is -γGlu-.

25. The compound according to embodiment 22, wherein -Z3-Z4- is -Ado-Ado-.

26. The compound according to embodiment 22, wherein -Z3-Z4- is -ELys-ELys-.

27. The compound according to any of the embodiments 1-14, wherein the substituent is selected from the group consisting of:

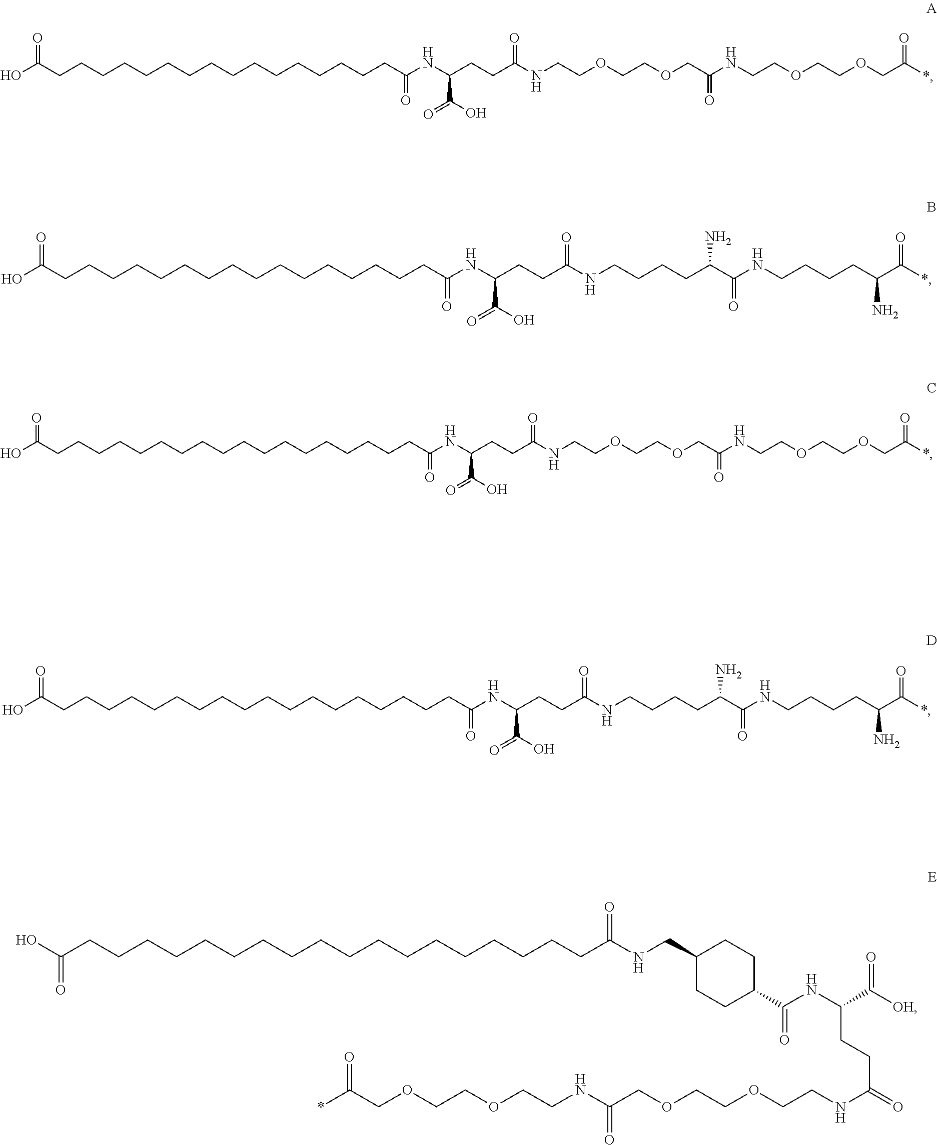

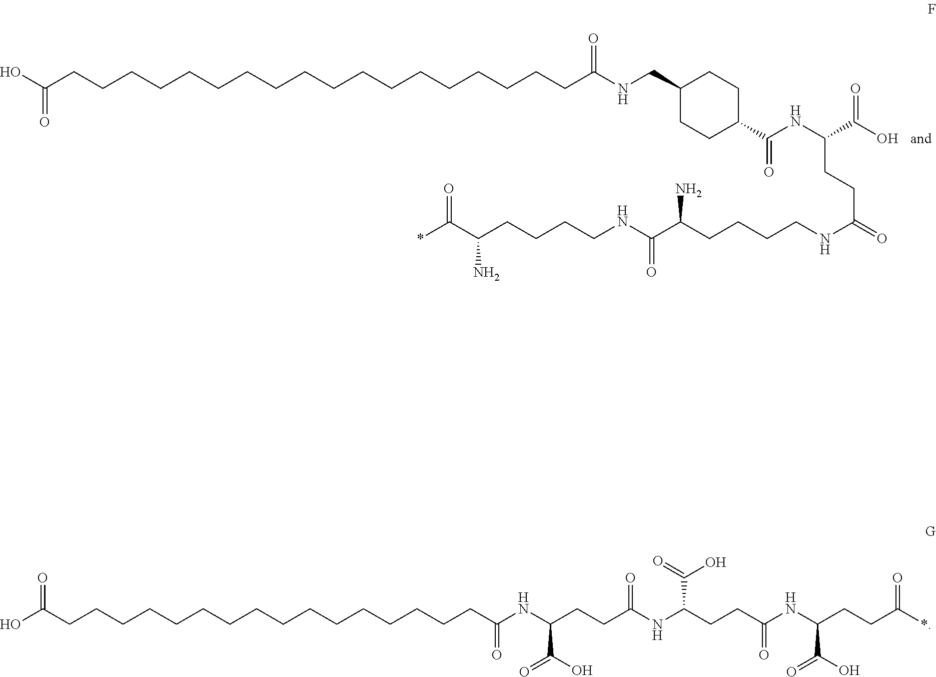

28. The compound according to embodiment 1, wherein the compound is selected from the group consisting of:

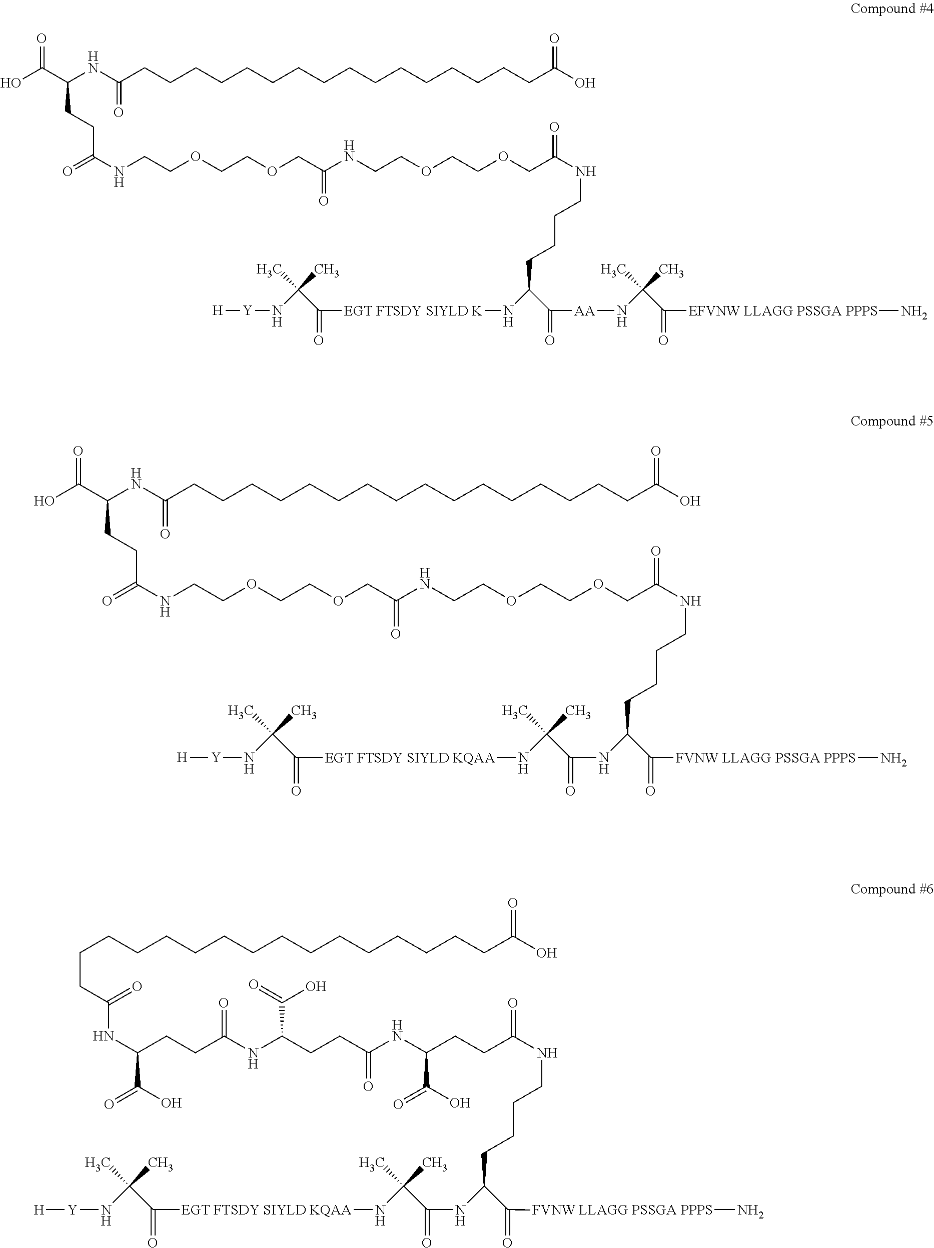

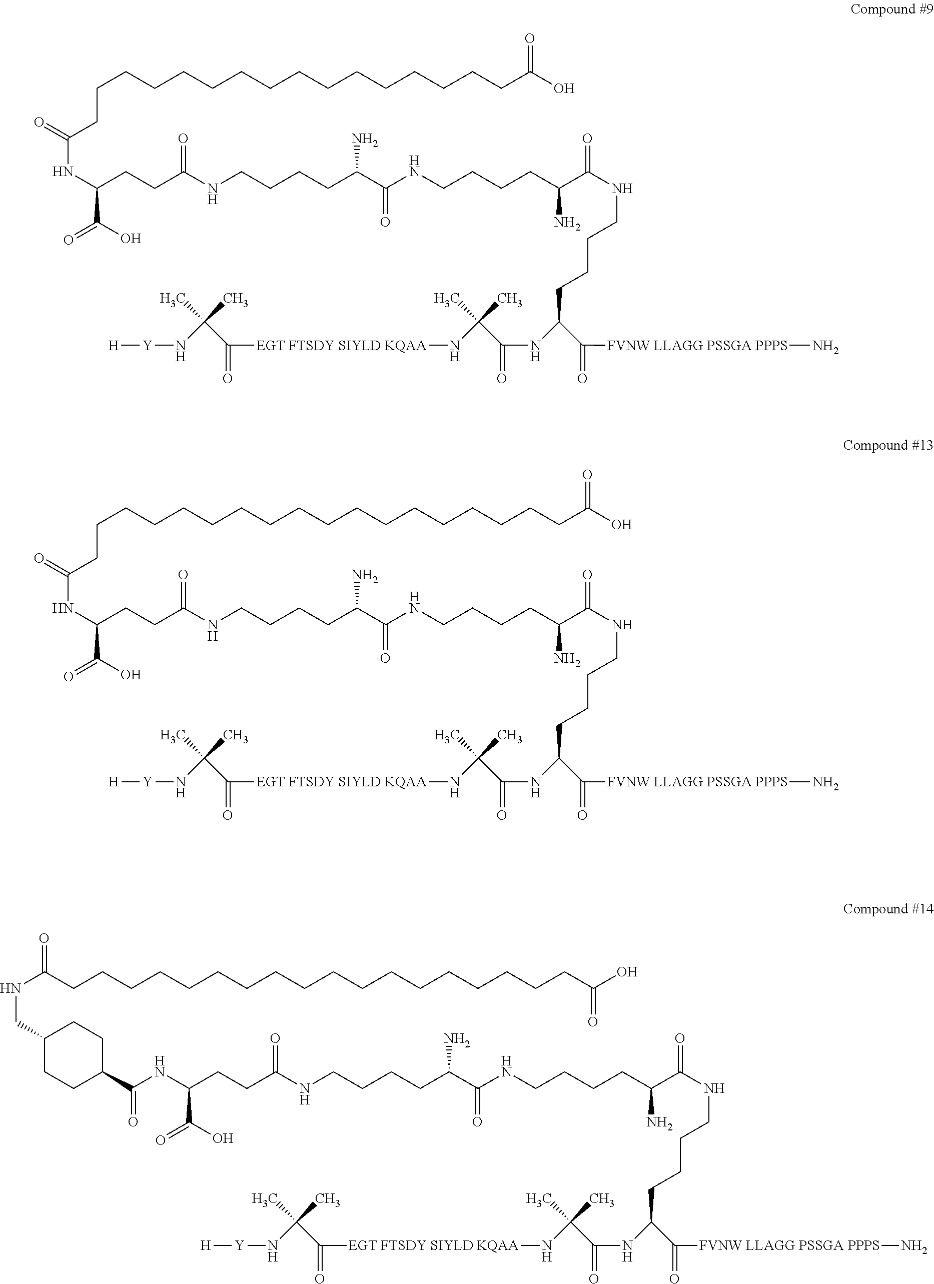

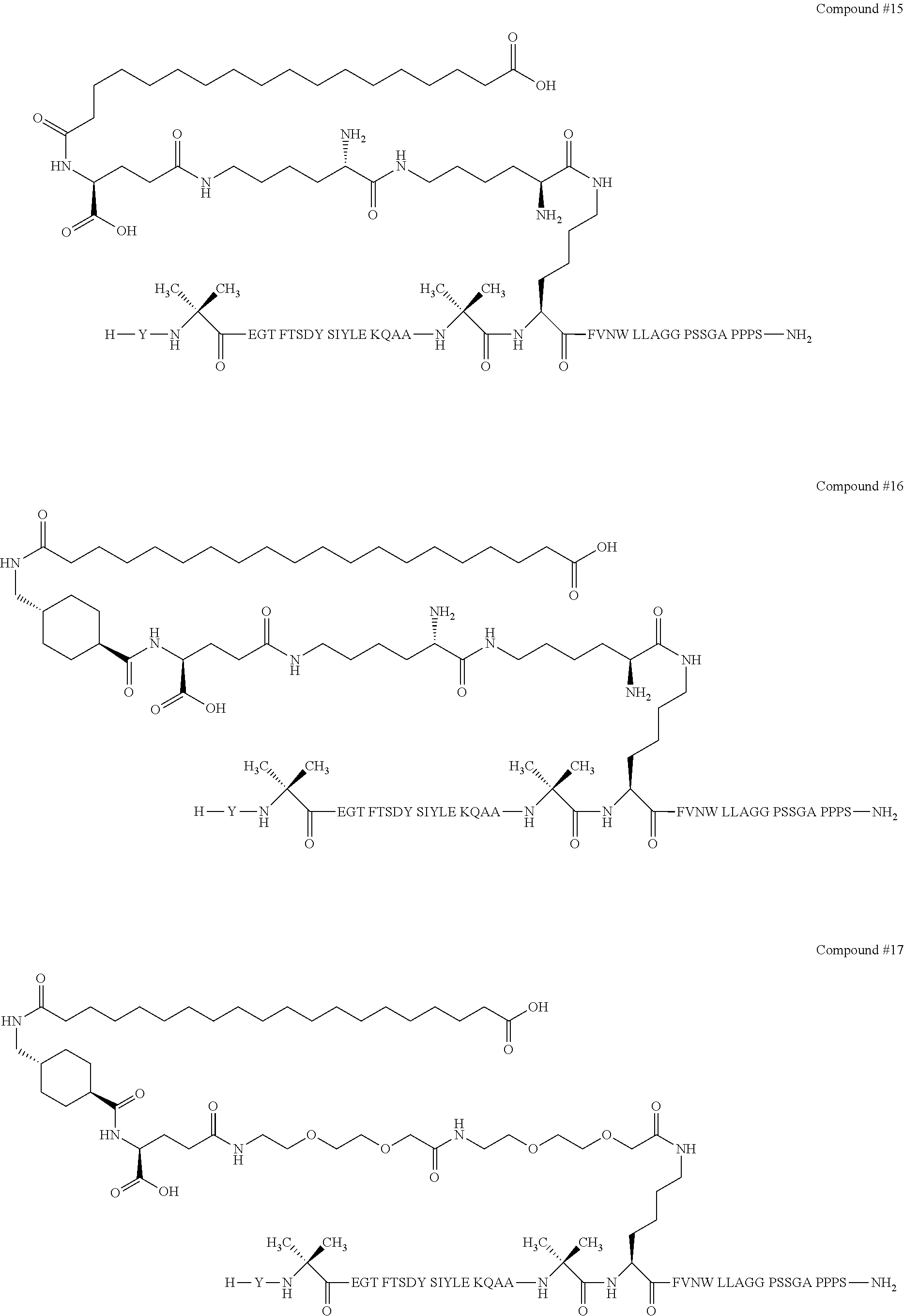

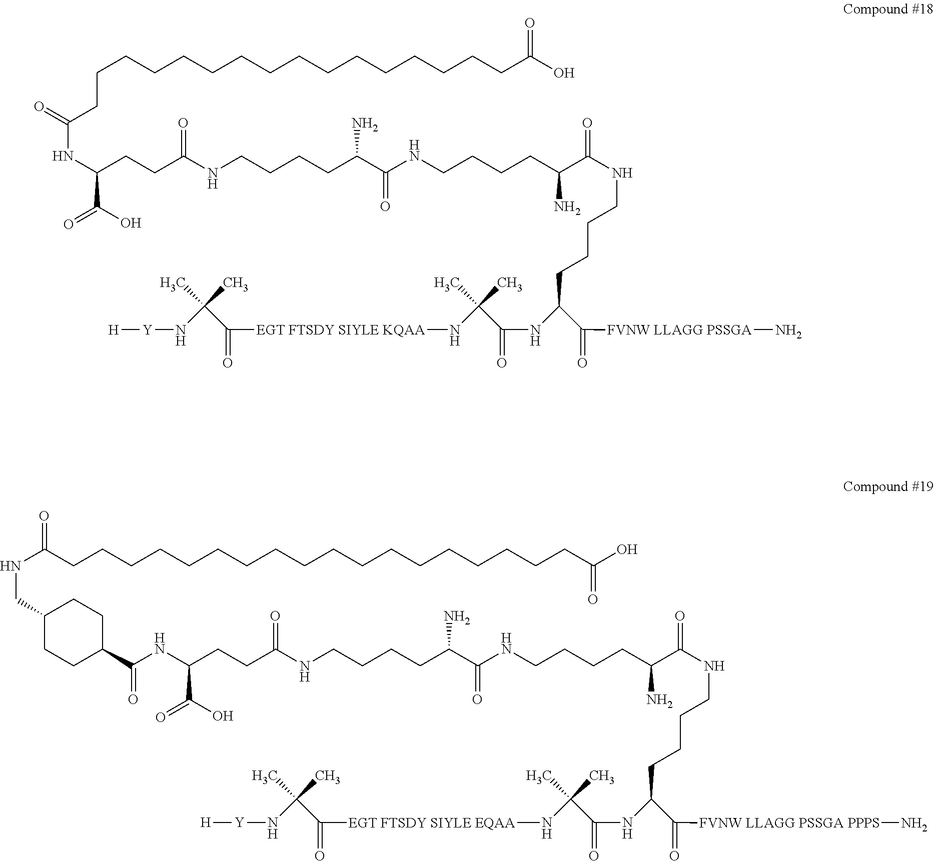

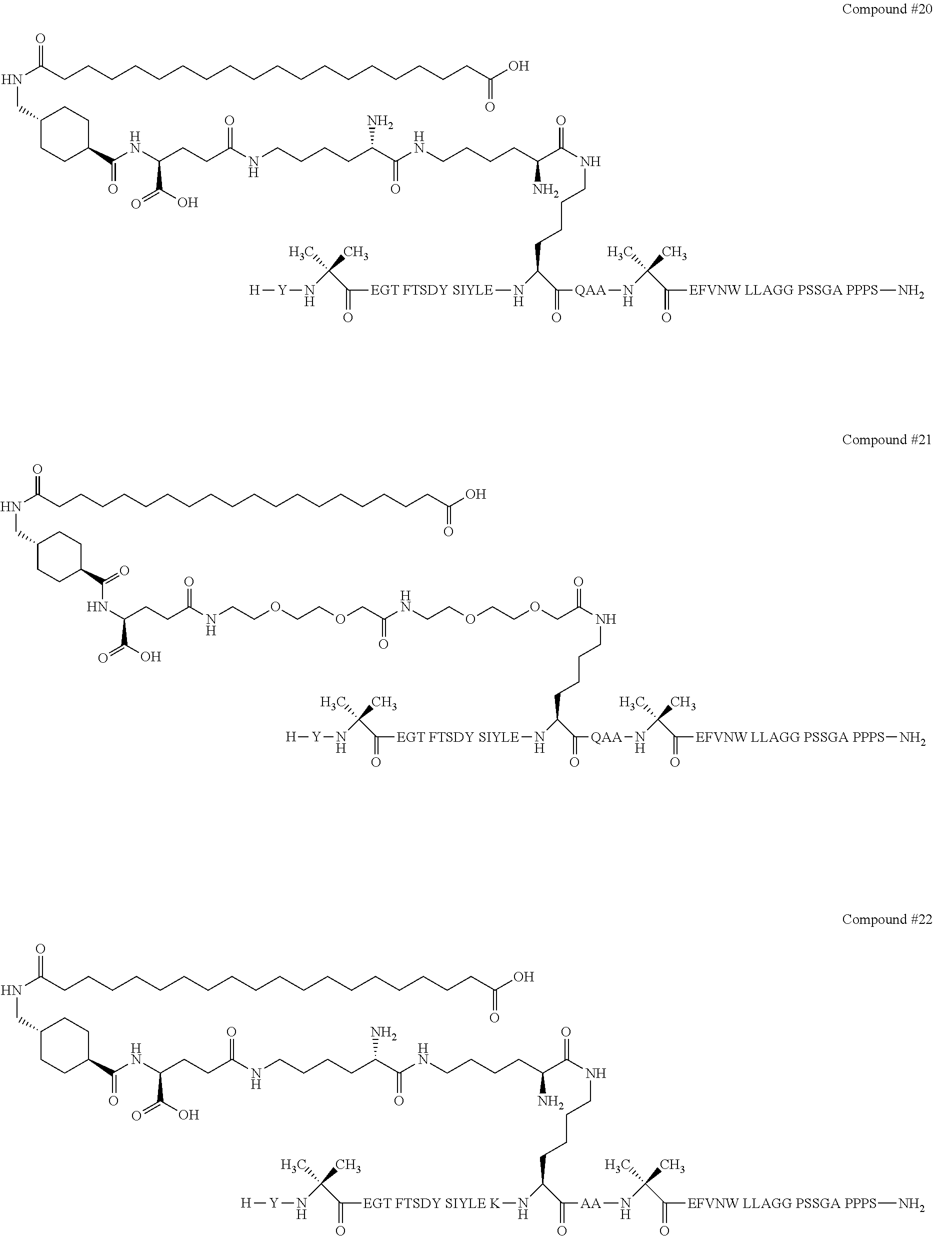

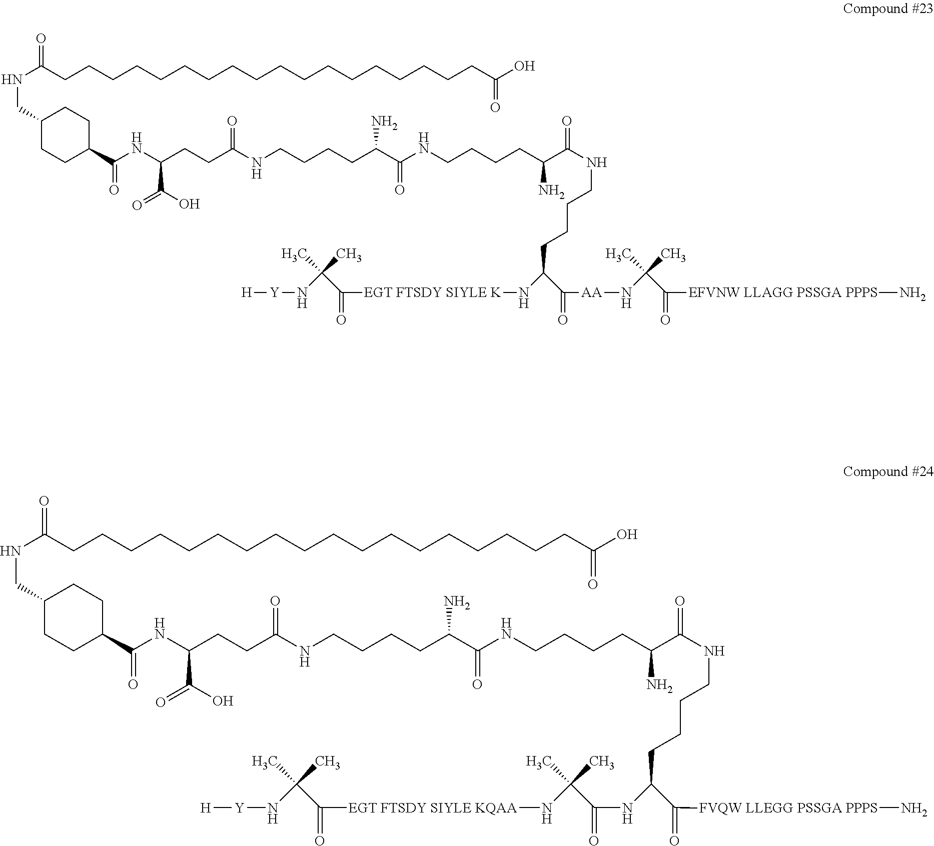

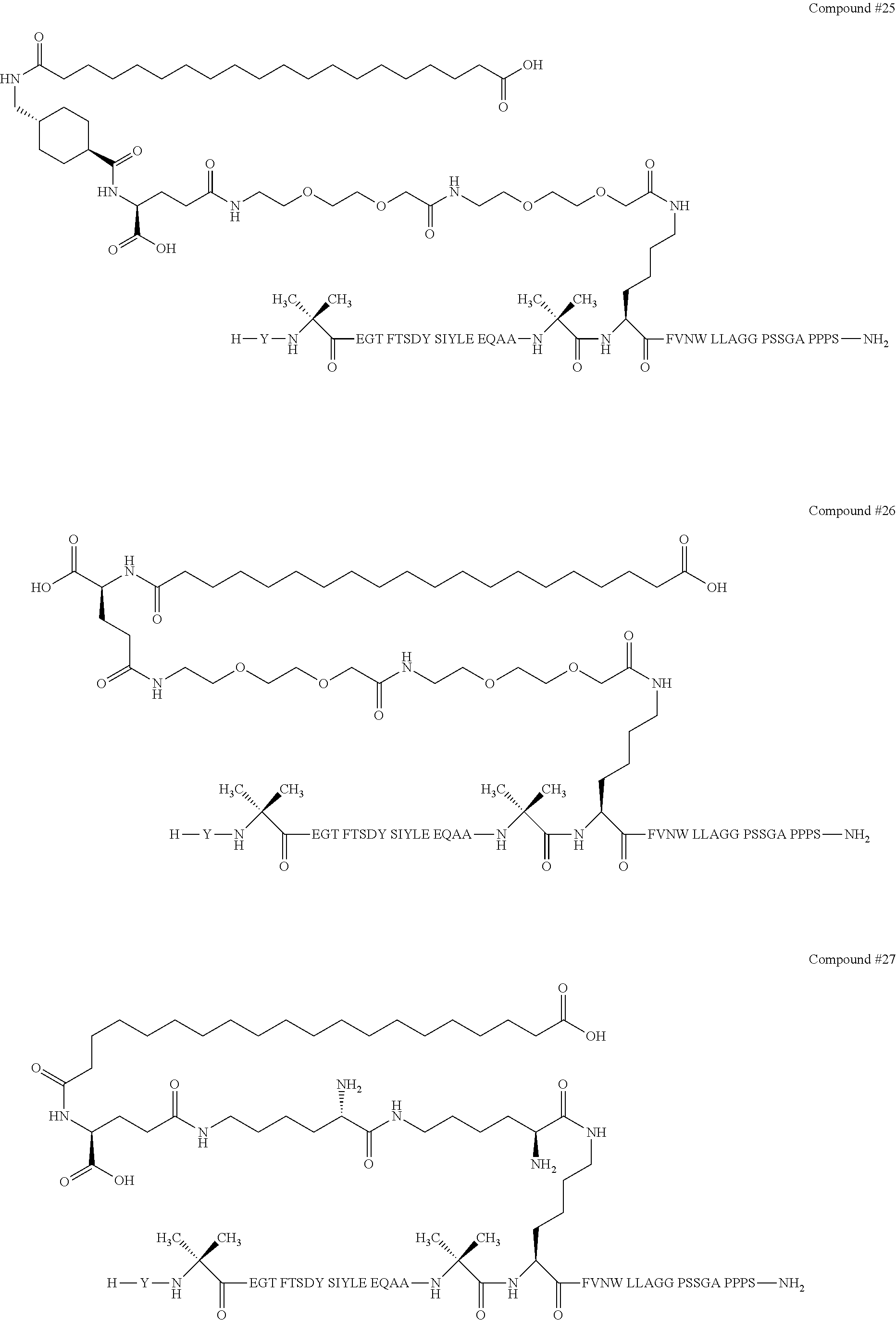

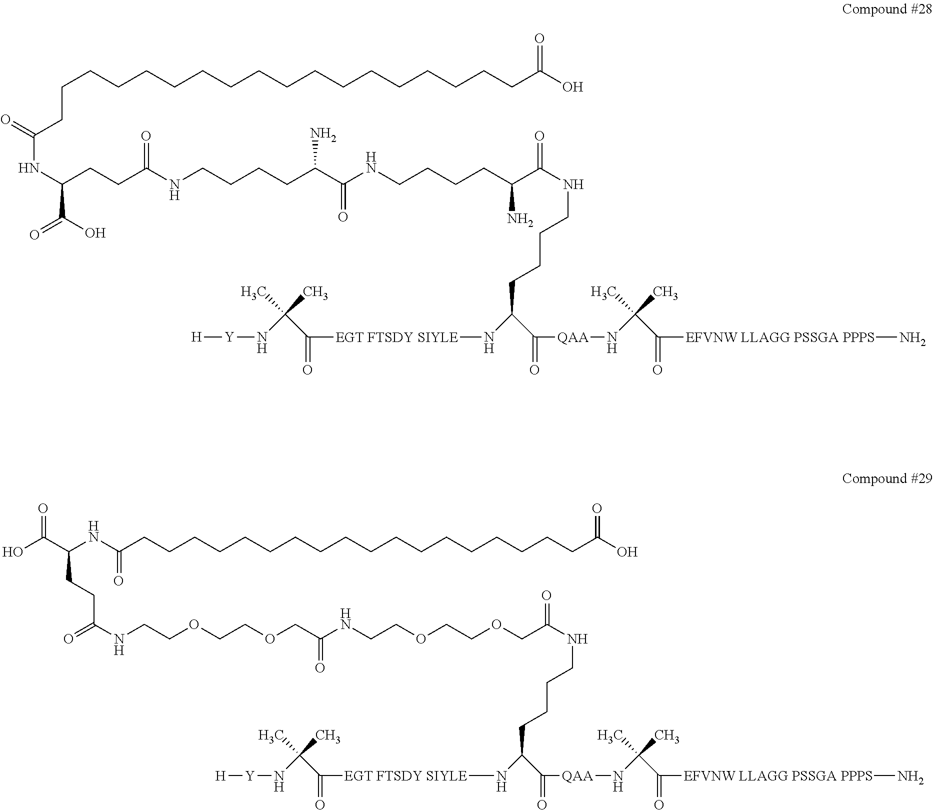

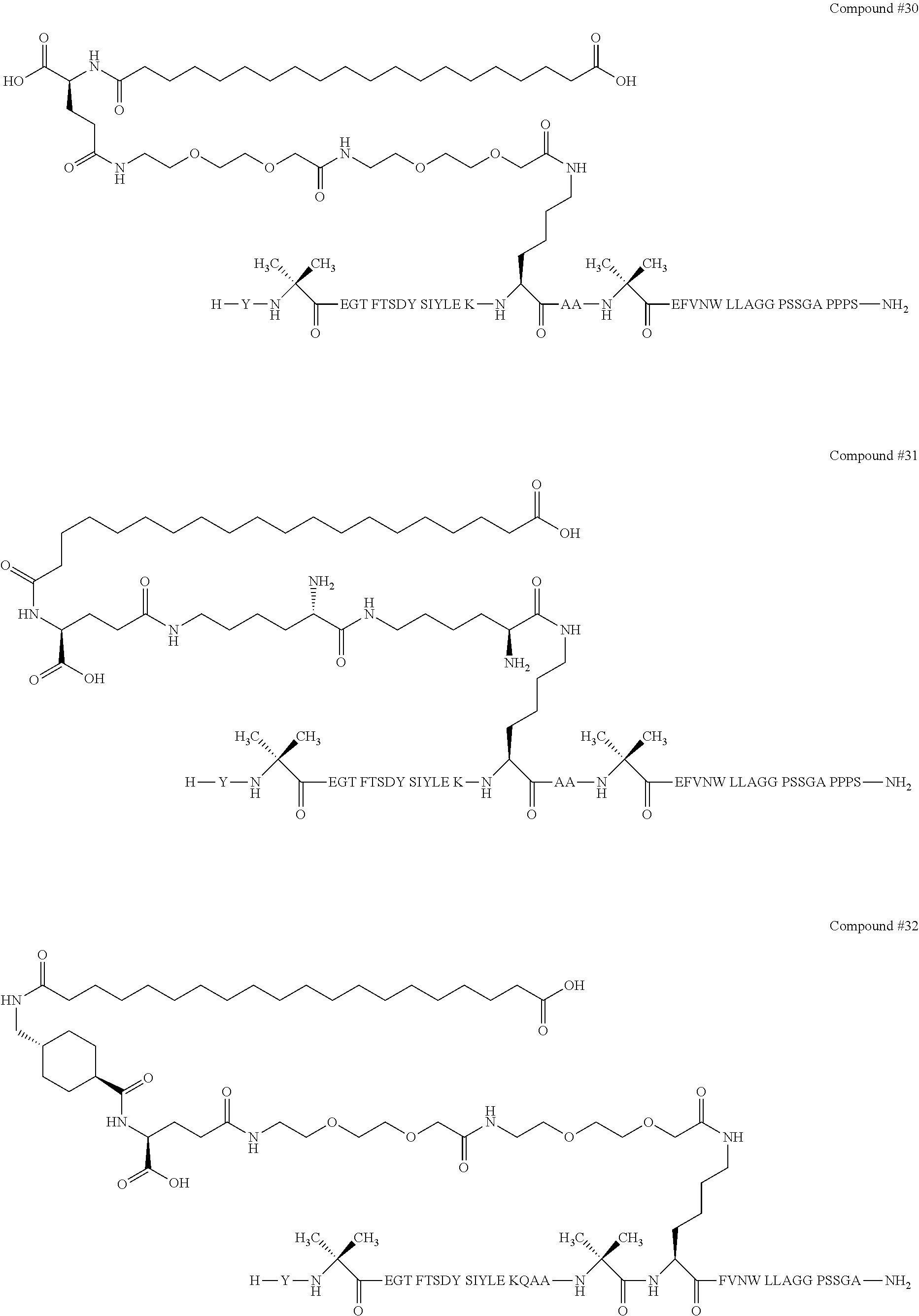

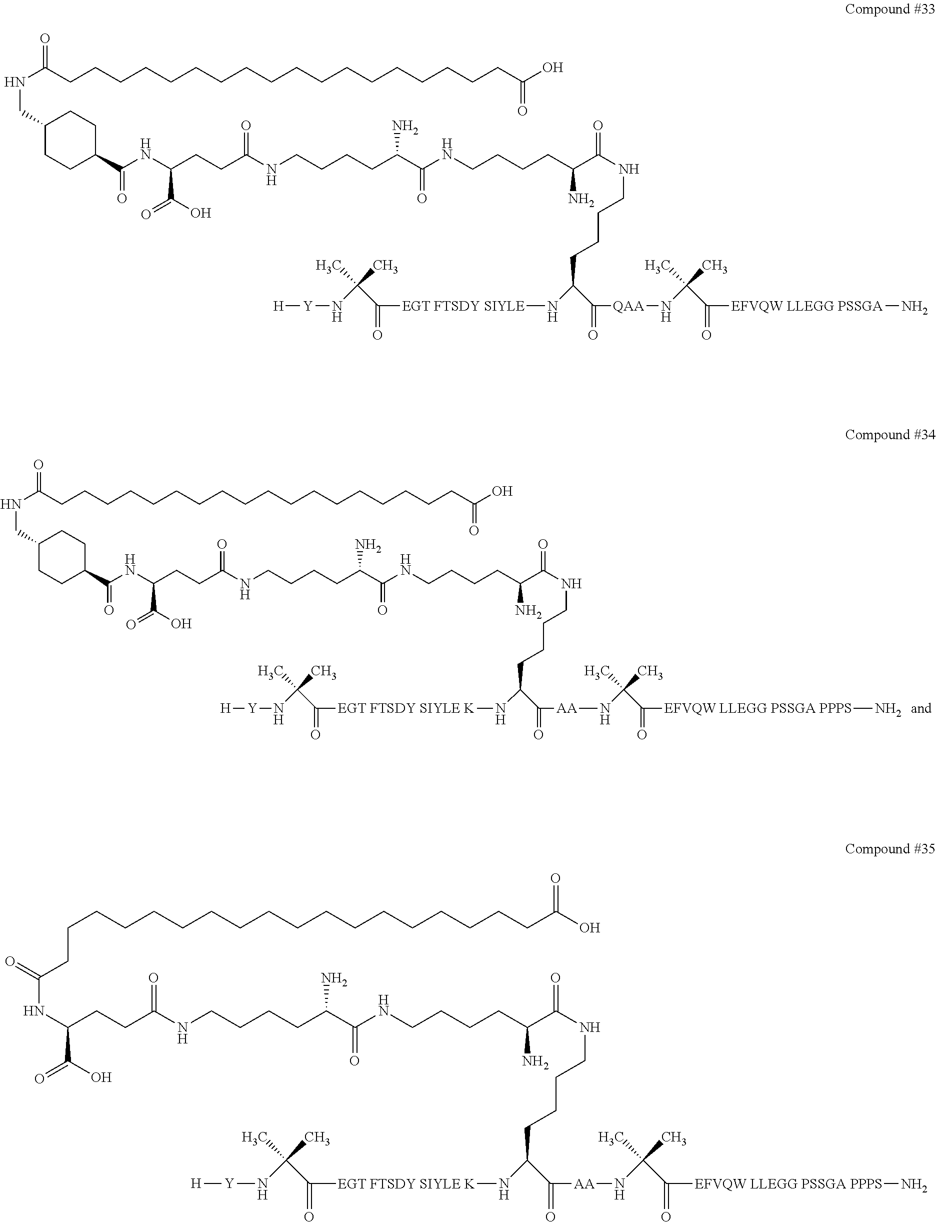

29. The compound according to embodiment 1, wherein the compound is selected from the group consisting of:

| | |
|---|---|
| 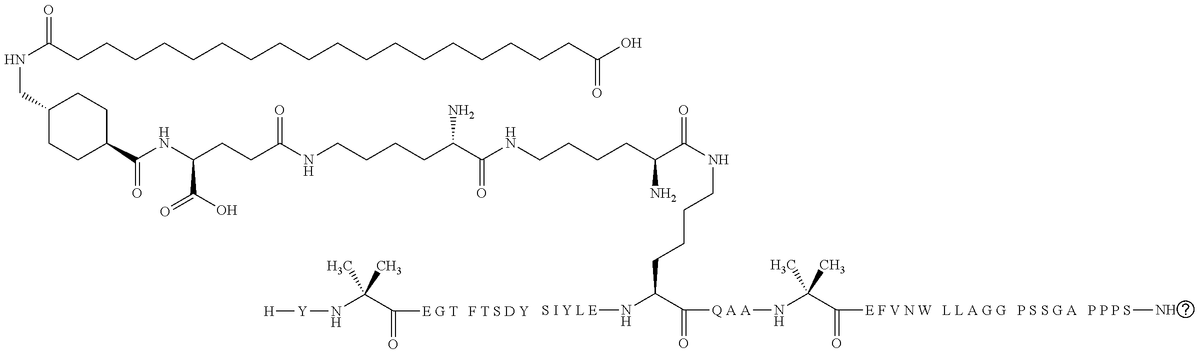 | Compound #20 |
| 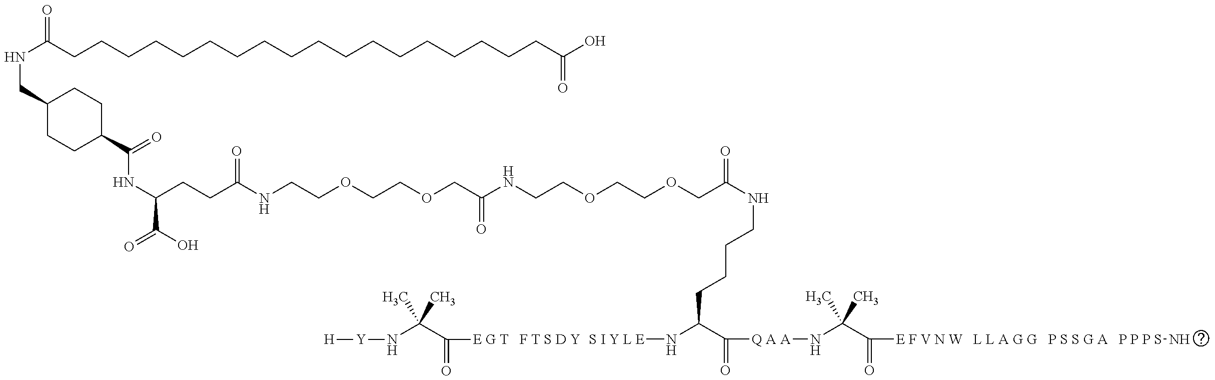 | Compound #21 |
| 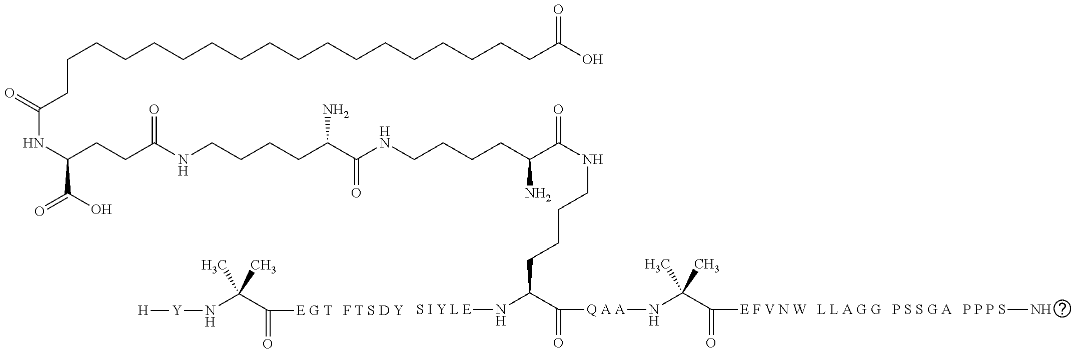 | Compound #28 |
| 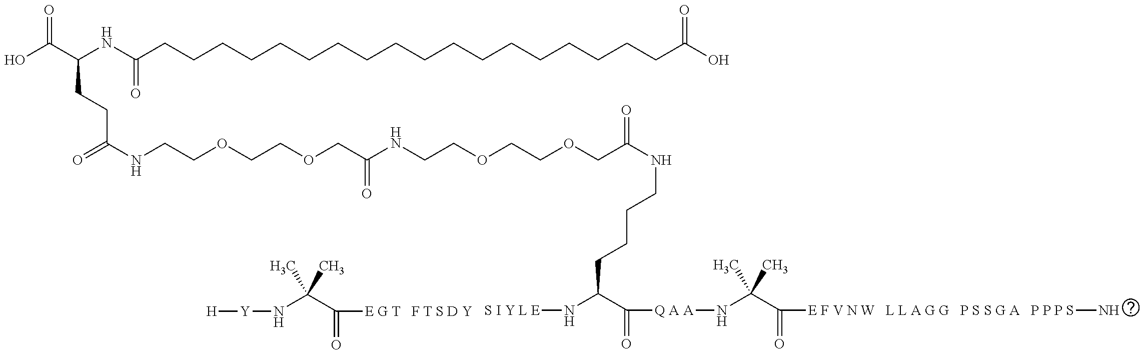 | Compound #29 |
| and | |
| 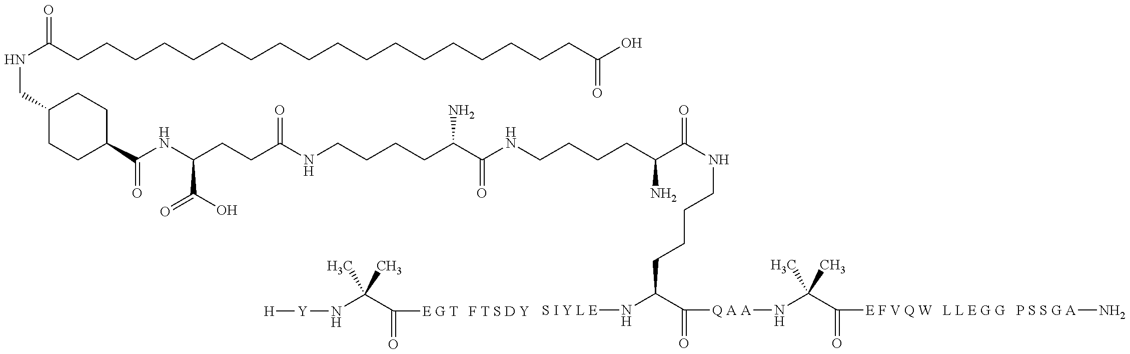 | Compound #33 |

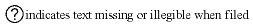

30. A compound according to any of the previous embodiments for use as a medicament.

31. A pharmaceutical composition comprising a compound according to any of the previous embodiments 0-29.

32. The composition according to embodiment 31, wherein said composition is an aqueous liquid formulation.

33. A pharmaceutical composition according to embodiment 31 and 32 for prevention and/or treatment of diabetes and/or obesity.

34. A pharmaceutical composition according to embodiment 31 and 32 for prevention and/or treatment of liver disorders, such as hepatic steatosis, non-alcoholic fatty liver disease (NAFLD), non-alcoholic steatohepatitis (NASH) liver inflammation and/or fatty liver.

35. A method for prevention and/or treatment of diabetes and/or obesity comprising administering to a patient a pharmaceutically active amount of the compound according to any one of embodiment 1-29.

36. A method for prevention and/or treatment of liver disorders, such as hepatic steatosis, non-alcoholic fatty liver disease (NAFLD), non-alcoholic steatohepatitis (NASH) liver inflammation and/or fatty liver comprising administering to a patient a pharmaceutically active amount of the compound according to any one of embodiment 1-29.

37. A peptide having the amino acid sequence:

(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉,

with an optional amide modification of the C-terminus wherein
   X₂ is Aib
   X₁₅ is D or E
   X₁₆ is E or K
   X₁₇ is Q or K
   X₂₀ is Aib
   X₂₁ is E or K
   X₂₄ is N or Q
   X₂₈ is A or E
   X₃₂ is S or absent
   X₃₃ is S or absent
   X₃₄ is G or absent
   X₃₅ is A or absent
   X₃₆ is P or absent
   X₃₇ is P or absent
   X₃₈ is P or absent
   X₃₉ is S or absent.

38. The peptide according to embodiment 37, wherein X₃₆, X₃₇, X₃₈ and X₃₉ are absent.

39. The peptide according to embodiment 37, wherein X₃₂X₃₃X₃₄X₃₅ is SSGA.

40. The peptide according to embodiment 37, wherein the peptide has the amide modification of the C-terminus.

41. The peptide according to embodiment 37, wherein the amino acid sequence of the peptide is

(SEQ ID NO.: 16)
YX₂EGTFTSDYSIYLX₁₅X₁₆X₁₇AAX₂₀X₂₁FVX₂₄WLLX₂₈GGPSSGA

wherein
X₂ is Aib
X₁₅ is D or E
X₁, is E or K
X₁₇ is Q or K
X₂₀ is Aib
X₂₁ is E or K
X₂₄ is N or Q
X₂₈ is A or E.

42. The peptide according to embodiment 37, wherein the amino acid sequence of the peptide is any one of SEQ ID NO.: 2, 3, 7, 8, 9, 10, 11, 12, 13 and 14.

43. The peptide according to embodiment 37, wherein the peptide has the amide modification of the C-terminus.

44. The peptide according to any of the previous embodiments 37-43, wherein X₁₆X₁₇AAX₂₀X₂₁ is selected from the group consisting of: KQAAAibE, KKAAAibE, KQAAAibK and EQAAAibK.

45. The peptide according to any of the previous embodiments 37-44, wherein the peptide activates the human GLP-1 and GIP receptors in vitro with an EC₅₀ of less than 20 pM when measured without HSA in a CRE luciferase reporter assays as described in Example 2.

46. The peptide according to embodiment 37, wherein the amino acid sequence of the peptide is any one of SEQ ID NO.: 10 and 14.

47. The peptide according to any of the previous embodiments 37-45, wherein X₁₆ is K.

48. The peptide according to any of the previous embodiments 37-45, wherein X₁₇ is K.

49. The peptide according to any of the previous embodiments 37-45, wherein X₂₀ is K.

50. A method for preparing a compound according to any of the embodiments 1-29.

51. A method for preparing a peptide according to any of the embodiments 37-49.

### Methods and Examples

### List of Abbreviations

The following abbreviations are used in the following, in alphabetical order:
Ac: acetyl
Ado (also called OEG): 8-amino-3,6-dioxaoctanoic acid
Aib: α-aminoisobutyric acid
API: active pharmaceutical ingredient
API-ES: atmospheric pressure ionization—electrospray
BHK: baby hamster kidney
Boc: tert-butyloxycarbonyl
BW: body weight
Cl-HOBt: 6-chloro-1-hydroxybenzotriazole
DCM: dichloromethane
DIC: diisopropylcarbodiimide
DIPEA: N,N-diisopropylethylamine
DMEM: Dulbecco's Modified Eagle's Medium
DPBS: Dulbecco's phosphate buffered saline
EDTA: ethylenediaminetetraacetic acid
ELISA: enzyme linked immunosorbent assay
equiv: molar equivalent
FBS: fetal bovine serum
Fmoc: 9-fluorenylmethyloxycarbonyl
GcgR: glucagon receptor
GIP: glucose-dependent insulinotropic polypeptide
GIPR: glucose-dependent insulinotropic polypeptide receptor
GLP-1: glucagon-like peptide 1
GLP-1R: glucagon-like peptide 1 receptor
h: hours
HEPES: 4-(2-hydroxyethyl)-1-piperazineethanesulfonic acid
HFIP: 1,1,1,3,3,3-hexafluoro-2-propanol or hexafluoroisopropanol
hGcgR: human glucagon receptor
hGIPR: human glucose-dependent insulinotropic polypeptide receptor
hGLP-1R: human glucagon-like peptide 1 receptor
HPLC: high performance liquid chromatography
HSA: human serum albumin
iAUC: baseline-subtracted area under the curve
i.p.: intraperitoneal
IPGTT: intraperitoneal glucose tolerance test
i.v. intravenously
LCMS: liquid chromatography mass spectroscopy
MeCN: acetonitrile
mGIPR: mouse glucose-dependent insulinotropic polypeptide receptor
mGLP-1R: mouse glucagon-like peptide 1 receptor
mM: millimolar
mmol: millimoles
min: minutes
Mtt: 4-methyltrityl
MW: molecular weight
NMP: 1-methyl-pyrrolidin-2-one
OEG: 8-amino-3,6-dioxaoctanoic acid (also called Ado)
OtBu: tert-butyl ester
Oxyma Pure®: cyano-hydroxyimino-acetic acid ethyl ester
Pbf: 2,2,4,6,7-pentamethyldihydrobenzofuran-5-sulfonyl
PBS: phosphate buffered saline
PK: pharmacokinetic
pM: picomolar
RP: reverse phase
rpm: rounds per minute
Rt: retention time
s.c.: subcutaneous
SEM: standard error of the mean
SPPS: solid phase peptide synthesis
tBu: tert-butyl
TFA: trifluoroacetic acid
TIS: triisopropylsilane
Trt: triphenylmethyl or trityl
Trx: tranexamic acid

### General Methods of Preparation

Methods for solid phase peptide synthesis (SPPS methods, including methods for de-protection of amino acids, methods for cleaving the peptide from the resin, and for its purification), as well as methods for detecting and characterising the resulting peptide (LCMS methods) are described here below.

Resins employed for the preparation of C-terminal peptide amides were H-Rink Amide-ChemMatrix resin (loading e.g. 0.5 mmol/g). The Fmoc-protected amino acid derivatives used, unless specifically stated otherwise, were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Fmoc-Aib-OH, Fmoc-D-Tyr-(tBu)-OH, etc. supplied from e.g. AAPPTEC, Anaspec, Bachem, ChemImpex, Iris Biotech, Midwest Biotech, Gyros Protein Technologies or Novabiochem. Where nothing else is specified, the natural L-form of the amino acids are used. When the N-terminal amino acid was not acetylated, the N-terminal amino acid was Boc protected at the alpha-amino group, either by using a reagent with the Boc group pre-installed (e.g. Boc-Tyr(tBu)-OH for peptides with Tyr at the N-terminus) or by exchanging the N-terminal Fmoc protective group for the Boc protective group after installation of the amino acid at the peptide N-terminus.

In case of modular albumin binding moiety attachment using SPPS, the following suitably protected building blocks such as but not limited to Fmoc-8-amino-3,6-dioxaoctanoic acid (Fmoc-Ado-OH), Fmoc-tranexamic acid (Fmoc-Trx-OH), Boc-Lys(Fmoc)-OH, Fmoc-Glu-OtBu, octadecanedioic acid mono-tert-butyl ester, nonadecanedioic acid mono-tert-butyl ester, eicosanedioic acid mono-tert-butyl ester, tetradecanedioic acid mono-tert-butyl ester, or 4-(9-carboxynonyloxy) benzoic acid tert-butyl ester were used. All operations stated below were performed within a 0.1-0.2 mmol synthesis scale range.

### 1. Synthesis of Resin-Bound Protected Peptide Backbone:

### Method: SPPS_A

SPPS was performed using Fmoc based chemistry on a Protein Technologies SymphonyX solid phase peptide synthesizer, using the manufacturer supplied protocols with minor modifications. Mixing was accomplished by occasional bubbling with nitrogen. The step-wise assembly was performed using the following steps: 1) pre-swelling of resin in DMF; 2) Fmoc-deprotection by the use of 20% (v/v) piperidine in DMF for two treatments of 10 min each; 3) washes with DMF to remove piperidine; 4) coupling of Fmoc-amino acid by the addition of Fmoc-amino acid (12 equiv) and Oxyma Pure® (12 equiv) as a 0.6 M solution each in DMF, followed by addition of DIC (12 equiv) as a 1.2 M solution in DMF, followed by the addition of DMF to reduce the final concentration of each component to 0.3 M, then mixing for 0.5-4 h; 4) washes with DMF to remove excess reagents; 5) final washes with DCM at the completion of the assembly. Some amino acids such as, but not limited to, those following a sterically hindered amino acid (e.g. Aib) were coupled with an extended reaction time (e.g. 4 h) to ensure reaction completion. For peptides bearing acetylation on the α-amine of the N-terminal amino acid, the N-terminal Fmoc group was removed by treatment with 20% (v/v) piperidine in DMF as described above in step 2. Then the peptidyl resin was removed from the synthesizer and manually treated with 10% (v/v) acetic anhydride/10% (v/v) DIPEA in DMF for 30-60 min, then washed with DMF and DCM.

### Method: SPPS_B

The protected peptidyl resin was synthesized according to the Fmoc strategy on an Applied Biosystems 431A solid-phase peptide synthesizer using the manufacturer supplied general Fmoc protocols. Mixing was accomplished by vortexing and occasional bubbling with nitrogen. The step-wise assembly was done using the following steps: 1) activation of Fmoc-amino acid by dissolution of solid Fmoc-acid acid (10 equiv) in Cl-HOBt (10 equiv) as a 1 M solution in NMP, then addition of DIC (10 equiv) as a 1 M solution in NMP, then mixing simultaneous to steps 2-3; 2) Fmoc-deprotection by the use of 20% (v/v) piperidine in NMP for one treatment of 3 min then a second treatment of 15 min; 3) washes with NMP to remove piperidine; 4) addition of activated Fmoc-amino acid solution to resin, then mixing for 45-90 min; 4) washes with NMP to remove excess reagents; 5) final washes with DCM at the completion of the assembly. The standard protected amino acid derivatives listed above were supplied in pre-weighed cartridges (from e.g. Midwest Biotech), and non-standard derivatives were weighed by hand. Some amino acids such as, but not limited to, those following a sterically hindered amino acid (e.g. Aib) were “double coupled” to ensure reaction completion, meaning that after the first coupling (e.g. 45 min) the resin is drained, more reagents are added (Fmoc-amino acid, DIC, Cl-HOBt), and the mixture allowed to react again (e.g. 45 min). For peptides bearing acetylation on the α-amine of the N-terminal amino acid, the N-terminal Fmoc group was removed by treatment with 20% (v/v) piperidine in NMP as described above in step 2. Then the peptidyl resin was removed from the synthesizer and manually treated with 10% (v/v) acetic anhydride/10% (v/v) pyridine in DMF for 30-60 min, then washed with DMF and DCM.

### 2. Attachment of Substituent to Resin-Bound Protected Peptide Backbone

### Method: SC_A

The N-epsilon-lysine protection Mtt protection group was removed by washing the resin with 30% HFIP in DCM for two treatments of 45 min each, following by washing with DCM and DMF. Acylation was performed on a Protein Technologies SymphonyX solid phase peptide synthesizer using the protocols described in method SPPS_A using stepwise addition of building blocks, such as, but not limited to, Boc-Lys(Fmoc)-OH, Fmoc-8-amino-3,6-dioxaoctanoic acid, Fmoc-tranexamic acid, Fmoc-Glu-OtBu, octadecanedioic acid mono-tert-butyl ester, and eicosanedioic acid mono-tert-butyl ester.

### Method: SC_B

The N-epsilon-lysine protection Mtt protection group was removed by washing the resin with 30% HFIP in DCM for two treatments of 45 min each, following by washing with DCM and DMF. Acylation was performed on an Applied Biosystems 431A solid-phase peptide synthesizer using the protocols described in method SPPS_B using stepwise addition of building blocks, such as, but not limited to, Boc-Lys(Fmoc)-OH, Fmoc-8-amino-3,6-dioxaoctanoic acid, Fmoc-tranexamic acid, Fmoc-Glu-OtBu, octadecanedioic acid mono-tert-butyl ester, and eicosanedioic acid mono-tert-butyl ester.

### 3. Cleavage of Resin Bound Peptide and Purification:

### Method: CP_A

Following completion of the sidechain synthesis, the peptidyl resin was washed with DCM and dried, then treated with TFA/water/TIS (95:2.5:2.5 v/v/v) for approximately 2 h, followed by precipitation with diethyl ether. The precipitate was washed with diethyl ether, dissolved in a suitable solvent (e.g. 2:1 water/MeCN), and let stand until all labile adducts decomposed. Purification was performed by reversed-phase preparative HPLC (Waters 2545 binary gradient module, Waters 2489 UV/Visible detector, Waters fraction collector Ill) on a Phenomenex Luna C8(2) column (10 μM particle size, 100 Å pore size, 250×21.2 mm dimensions). Separation of impurities and product elution was accomplished using an increasing gradient of MeCN in water containing 0.1% TFA. Relevant fractions were checked for identity and purity by analytical LCMS. Fractions containing the pure desired product were pooled and freeze-dried to afford the peptide TFA salt as a white solid.

4. Salt Exchange from TFA to Sodium Salt:

### Method: SX_A

The freeze-dried peptide isolated from method CP_A was dissolved to 5-20 mg/mL in an appropriate aqueous buffer (e.g. 4:1 water/MeCN, 0.2 M sodium acetate) and adjusted to pH 7-8 with 1 M NaOH if necessary to achieve full solubility. The buffered solutions containing the peptide were salt-exchanged using a Sep-Pak C18 cartridge (0.5-2 g): The cartridge was first equilibrated with 4 column volumes of isopropanol, then 4 column volumes of MeCN, then 8 column volumes of water. The peptide solution was applied to the cartridge, and the flow through was reapplied to ensure complete retention of peptide. The cartridge was washed with 4 column volumes of water, then 10 column volumes of a buffer solution (e.g. pH 7.5) containing such as, but not limited to, NaHCO₃, NaOAc, or Na₂HPO₄. The column was washed with 4 column volumes of water, and the peptide was eluted with 5-20 column volumes of 50-80% MeCN in water. The peptide-containing eluent was freeze-dried to afford the peptide sodium salt as a white solid, which was used as such.

### General Methods of Detection and Characterisation

### LCMS Methods:

### Method: LCMS_A

LCMS_A was performed on a setup consisting of an Agilent 1260 Infinity series HPLC system and an Agilent Technologies 6120 Quadrupole MS. Eluents: A: 0.05% TFA in water; B: 0.05% TFA in 9:1 MeCN/water.

The analysis was performed at RT (column temp 37C) by injecting an appropriate volume of the sample onto the column which was eluted with a gradient of A and B. Column: Phenomenex Kinetex C8, 2.6 μm, 100 Å, 4.6×75 mm. Gradient run time: Linear 10-80% B over 10 min at a flow rate of 1.0 mL/min. Detection: diode array detector set to 214 nm. MS ionisation mode: API-ES, positive polarity. MS scan mass range: 500-2000 amu. The most abundant isotope of each m/z is reported.

### Method: LCMS_B

LCMS_B was performed on a setup consisting of an Agilent 1260 Infinity series HPLC system and an Agilent Technologies 6120 Quadrupole MS. Eluents: A: 0.05% TFA in water; B: 0.05% TFA in 9:1 MeCN/water.

The analysis was performed at RT (column temp 37C) by injecting an appropriate volume of the sample onto the column which was eluted with a gradient of A and B. Column: Phenomenex Kinetex C8, 2.6 μm, 100 Å, 4.6×75 mm. Gradient run time: Linear 20-100% B over 10 min at a flow rate of 1.0 mL/min. Detection: diode array detector set to 214 nm. MS ionisation mode: API-ES, positive polarity. MS scan mass range: 500-2000 amu. The most abundant isotope of each m/z is reported.

### Example 1: Synthesis of Compounds

The compounds are in the following described using single letter amino acid codes, except for Aib. The substituent is included after the lysine (K) residue to which it is attached.

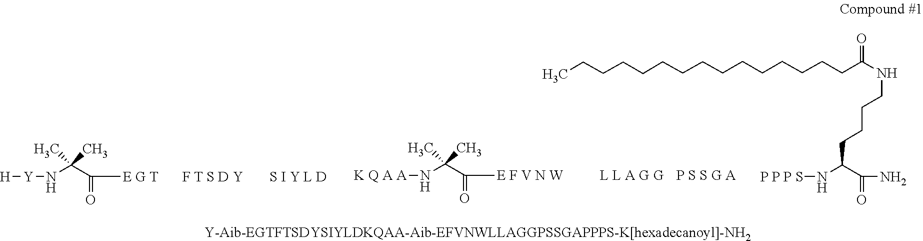

SEQ ID NO: 1 with substituent at position K40 and C-terminal amide modification.

Substituent: C16 monoacid also known as hexadecanoyl

Synthesis methods: SPPS_B; SC_B; CP_A

Molecular weight (average) calculated: 4473.0 Da

LCMS_B: Rt=7.1 min; found [M+3H]³⁺ 1491.7, [M+4H]⁴⁺ 1119.1

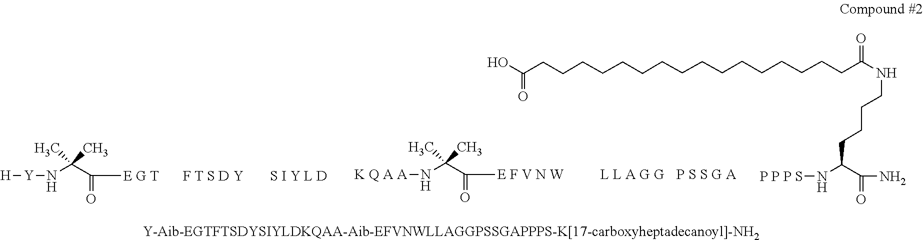

SEQ ID NO: 1 with substituent at position K40 and C-terminal amide modification.

Substituent: C18 diacid also known as 17-carboxyheptadecanoyl

Synthesis methods: SPPS_A; SC_A; CP_A

Molecular weight (average) calculated: 4531.1 Da

LCMS_B: Rt=6.5 min; found [M+3H]³⁺ 1511.0, [M+4H]⁴⁺ 1133.6 Compound #3

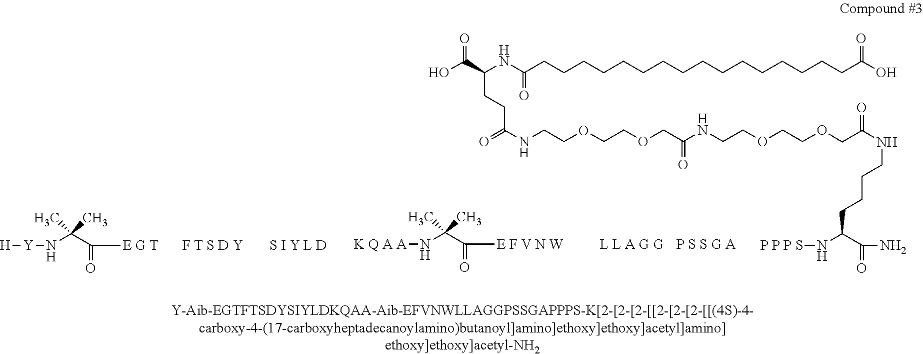

SEQ ID NO: 1 with substituent at position K40 and C-terminal amide modification.

Substituent: C18 diacid-γGlu-Ado-Ado (A)

Synthesis methods: SPPS_A; SC_A; CP_A

Molecular weight (average) calculated: 4950.5 Da

LCMS_B: Rt=6.1 min; found [M+3H]³⁺ 1651.0, [M+4H]⁴⁺ 1238.3

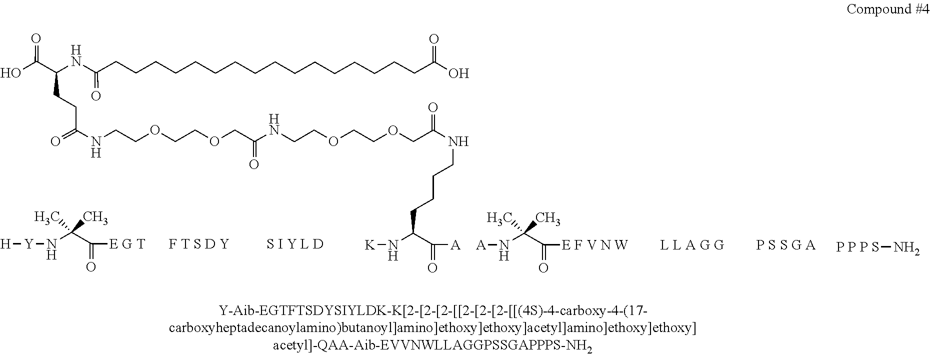

SEQ ID NO: 2 with substituent at position K17 and C-terminal amide modification.

Substituent: C18 diacid-γGlu-Ado-Ado (A)

Synthesis methods: SPPS_A; SC_B; CP_A

Molecular weight (average) calculated: 4822.4 Da

LCMS_B: Rt=6.1 min; found [M+3H]³⁺ 1608.3, [M+4H]⁴⁺ 1206.5 Compound #5

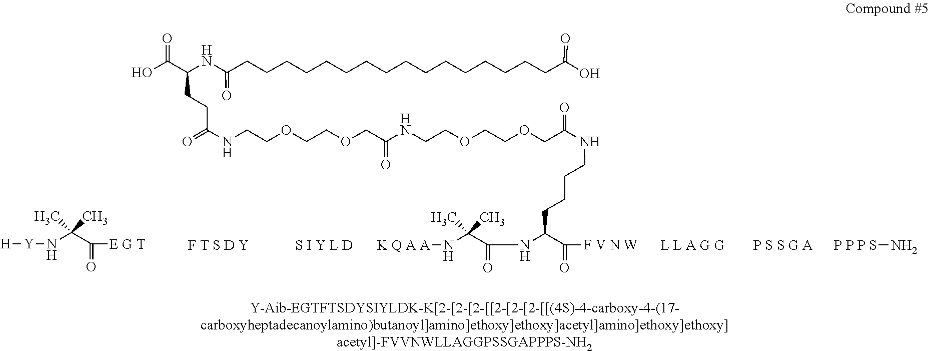

SEQ ID NO: 3 with substituent at position K21 and C-terminal amide modification.

Substituent: C18 diacid-γGlu-Ado-Ado (A)

Synthesis methods: SPPS_A; SC_B; CP_A

Molecular weight (average) calculated: 4821.4 Da

LCMS_B: Rt=6.0 min; found [M+3H]³⁺ 1607.9, [M+4H]⁴⁺ 1206.1

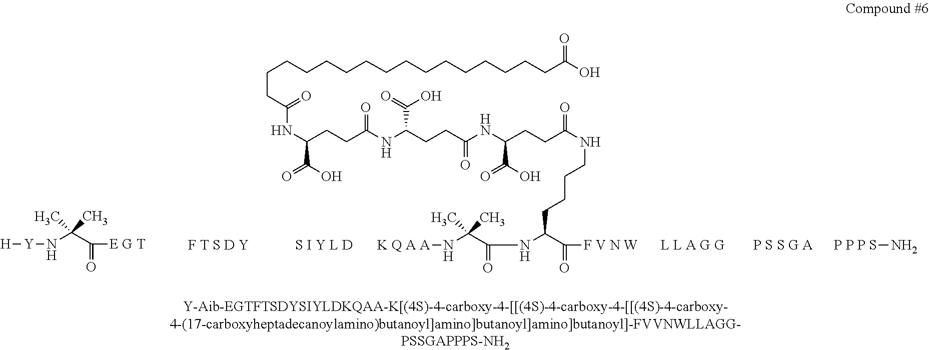

SEQ ID NO: 3 with substituent at position K21 and C-terminal amide modification.

Substituent: C18 diacid-γGlu-γGlu-γGlu (G)

Synthesis methods: SPPS_A; SC_B; CP_A

Molecular weight (average) calculated: 4789.3 Da

LCMS_A: Rt=7.9 min; found [M+3H]³⁺ 1597.1, [M+4H]⁴⁺ 1198.2

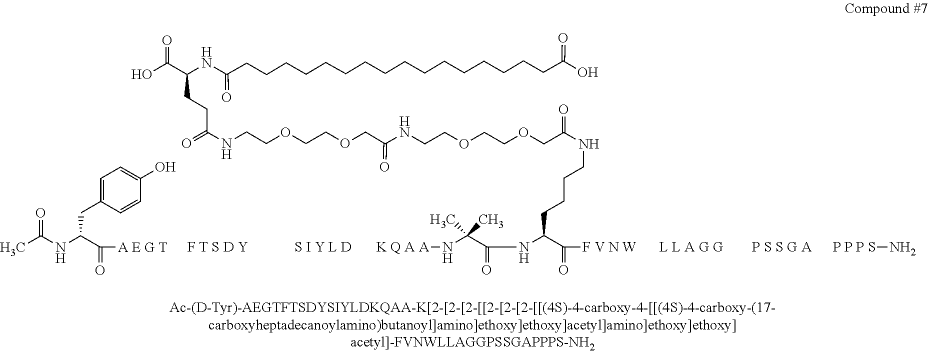

SEQ ID NO: 4 with substituent at position K21 and C-terminal amide modification.

Substituent: C18 diacid-γGlu-Ado-Ado (A)

Synthesis methods: SPPS_A; SC_A; CP_A

Molecular weight (average) calculated: 4849.4 Da

LCMS_A: Rt=8.3 min; found [M+3H]³⁺ 1617.1, [M+4H]⁴⁺ 1213.1

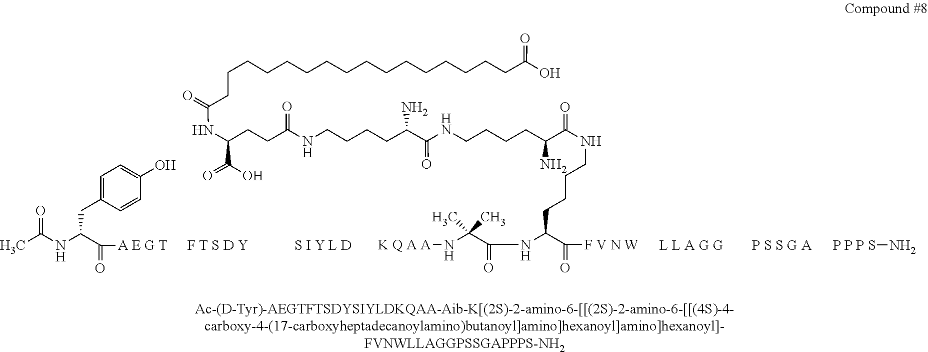

SEQ ID NO: 4 with substituent at position K21 and C-terminal amide modification.

Substituent: C18 diacid-γGlu-ELys-ELys (B)

Synthesis methods: SPPS_A; SC_B; CP_A

Molecular weight (average) calculated: 4815.4 Da

LCMS_A: Rt=7.9 min; found [M+3H]³⁺ 1605.9, [M+4H]⁴⁺ 1204.6

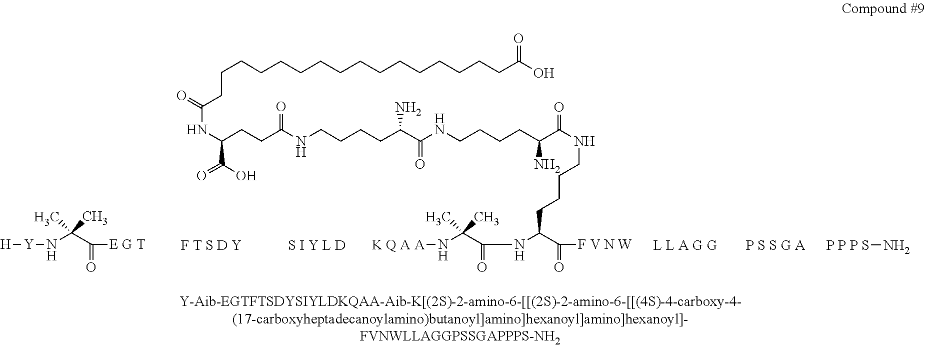

SEQ ID NO: 3 with substituent at position K21 and C-terminal amide modification.

Substituent: C18 diacid-γGlu-ELys-ELys (B)

Synthesis methods: SPPS_A; SC_B; CP_A

Molecular weight (average) calculated: 4787.4 Da

LCMS_A: Rt=7.7 min; found [M+3H]³⁺ 1596.5, [M+4H]⁴⁺ 1197.6

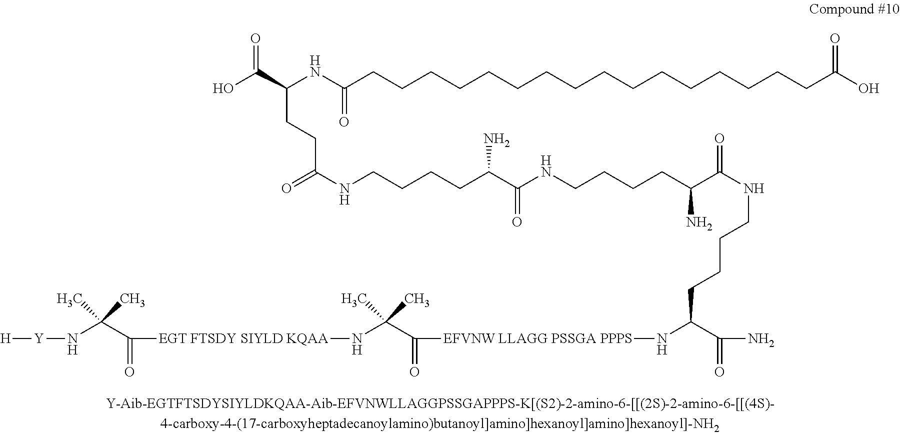

SEQ ID NO: 1 with substituent at position K40 and C-terminal amide modification.

Substituent: C18 diacid-γGlu-ELys-ELys (B)

Synthesis methods: SPPS_A; SC_B; CP_A

Molecular weight (average) calculated: 4916.5 Da

LCMS_A: Rt=7.7 min; found [M+3H]³⁺ 1639.5, [M+4H]⁴⁺ 1229.9

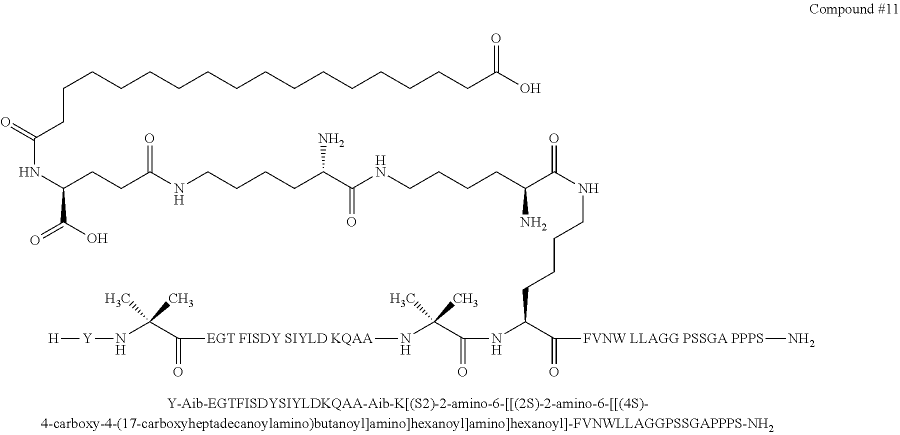

SEQ ID NO: 5 with substituent at position K21 and C-terminal amide modification.

Substituent: C18 diacid-γGlu-ELys-ELys (B)

Synthesis methods: SPPS_B; SC_B; CP_A

Molecular weight (average) calculated: 4799.5 Da

LCMS_A: Rt=8.0 min; found [M+3H]³⁺ 1600.5, [M+4H]⁴⁺ 1200.8

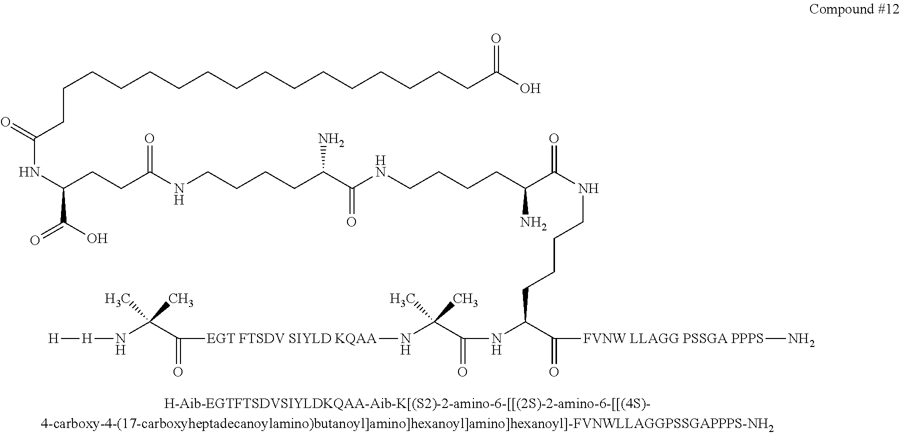

SEQ ID NO: 6 with substituent at position K21 and C-terminal amide modification.

Substituent: C18 diacid-γGlu-ELys-ELys (B)

Synthesis methods: SPPS_B; SC_B; CP_A

Molecular weight (average) calculated: 4697.3 Da

LCMS_A: Rt=7.5 min; found [M+3H]³⁺ 1566.6, [M+4H]⁴⁺ 1175.2

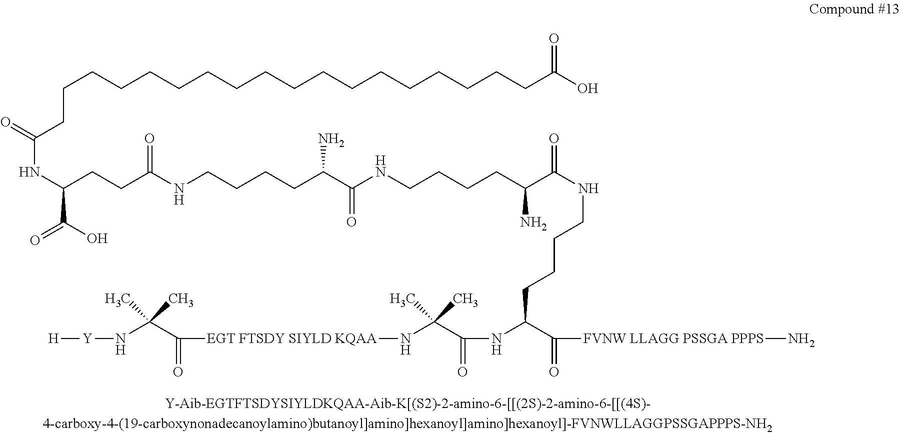

SEQ ID NO: 3 with substituent at position K21 and C-terminal amide modification.

Substituent: C20 diacid-γGlu-ELys-ELys (D)

Synthesis methods: SPPS_A; SC_B; CP_A

Molecular weight (average) calculated: 4815.5 Da

LCMS_A: Rt=8.0 min; found [M+3H]³⁺ 1605.8, [M+4H]⁴⁺ 1204.7

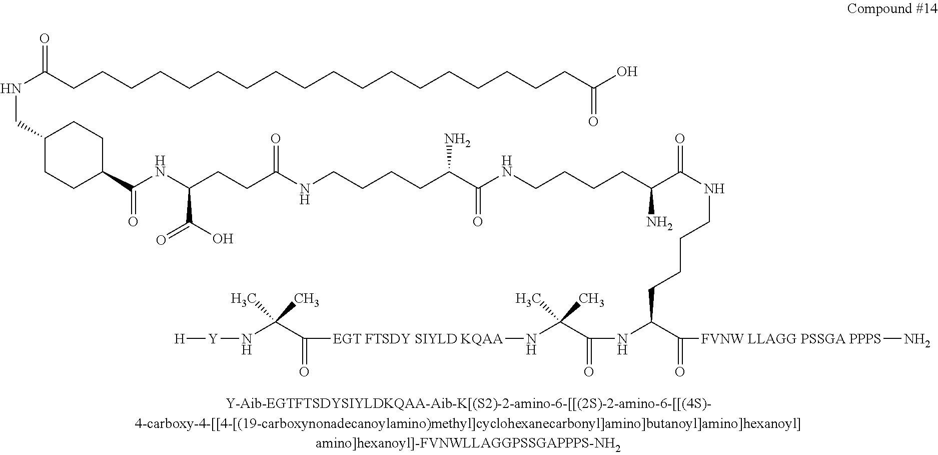

SEQ ID NO: 3 with substituent at position K21 and C-terminal amide modification.

Substituent: C20 diacid-Trx-γGlu-ELys-ELys (F)

Synthesis methods: SPPS_B; SC_B; CP_A

Molecular weight (average) calculated: 4954.7 Da

LCMS_A: Rt=8.3 min; found [M+3H]³⁺ 1652.3, [M+4H]⁴⁺ 1239.5

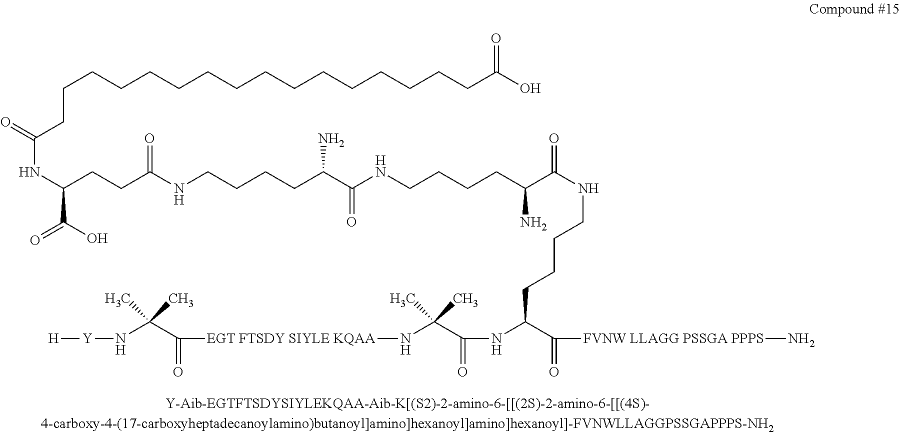

SEQ ID NO: 7 with substituent at position K21 and C-terminal amide modification.

Substituent: C18 diacid-γGlu-ELys-ELys (B)

Synthesis methods: SPPS_A; SC_B; CP_A

Molecular weight (average) calculated: 4801.5 Da

LCMS_A: Rt=7.7 min; found [M+3H]³⁺ 1601.2, [M+4H]⁴⁺ 1201.1

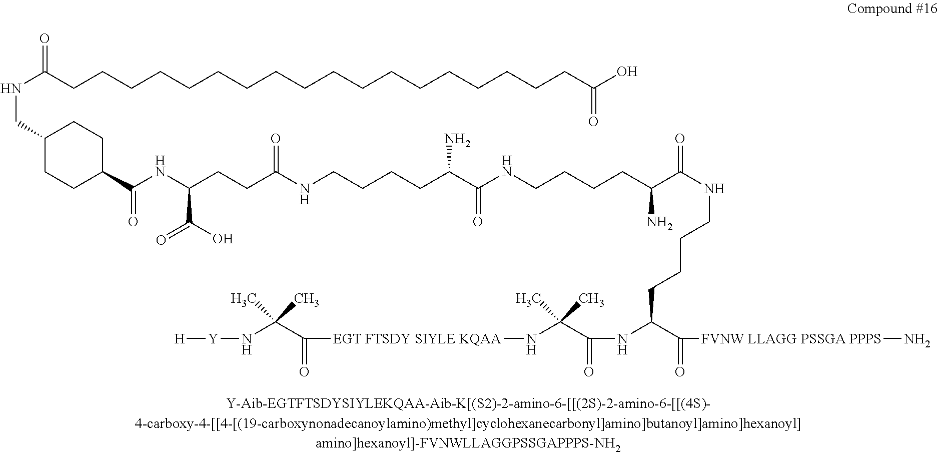

SEQ ID NO: 7 with substituent at position K21 and C-terminal amide modification.

Substituent: C20 diacid-Trx-γGlu-ELys-ELys (F)

Synthesis methods: SPPS_A; SC_B; CP_A

Molecular weight (average) calculated: 4968.7 Da

LCMS_A: Rt=8.3 min; found [M+3H]³⁺ 1657.1, [M+4H]⁴⁺ 1243.1

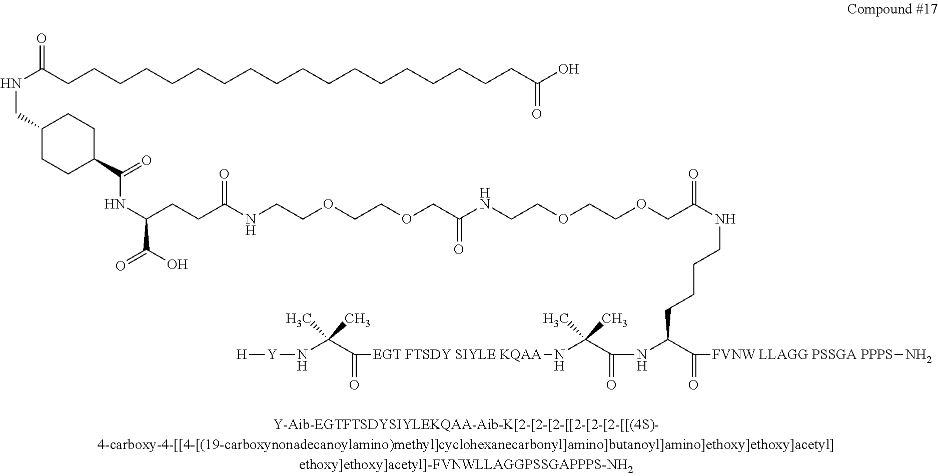

SEQ ID NO: 7 with substituent at position K21 and C-terminal amide modification.

Substituent: C20 diacid-Trx-γGlu-Ado-Ado (E)

Synthesis methods: SPPS_A; SC_B; CP_A

Molecular weight (average) calculated: 5002.7 Da

LCMS_B: Rt=6.5 min; found [M+3H]³⁺ 1668.3, [M+4H]⁴⁺ 1251.5

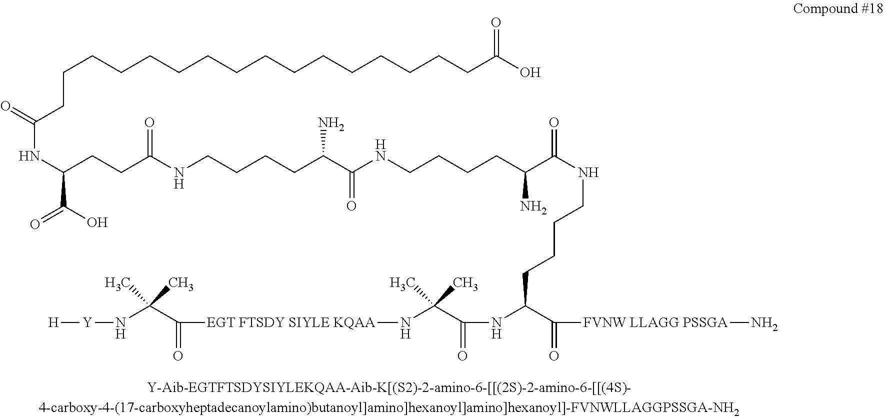

SEQ ID NO: 8 with substituent at position K21 and C-terminal amide modification.

Substituent: C18 diacid-γGlu-ELys-ELys (B)

Synthesis methods: SPPS_A; SC_B; CP_A

Molecular weight (average) calculated: 4423.0 Da

LCMS_A: Rt=7.9 min; found [M+3H]³⁺ 1475.0, [M+4H]⁴⁺ 1106.6

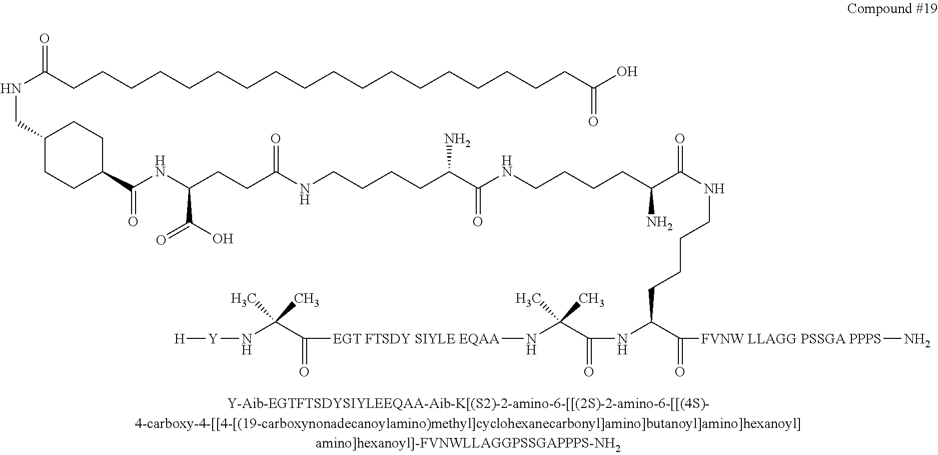

SEQ ID NO: 9 with substituent at position K21 and C-terminal amide modification.

Substituent: C20 diacid-Trx-γGlu-ELys-ELys (F)

Synthesis methods: SPPS_A; SC_B; CP_A; SX_A

Molecular weight (average) calculated: 4969.6 Da

LCMS_B: Rt=6.8 min; found [M+3H]³⁺ 1657.3, [M+4H]⁴⁺ 1243.0

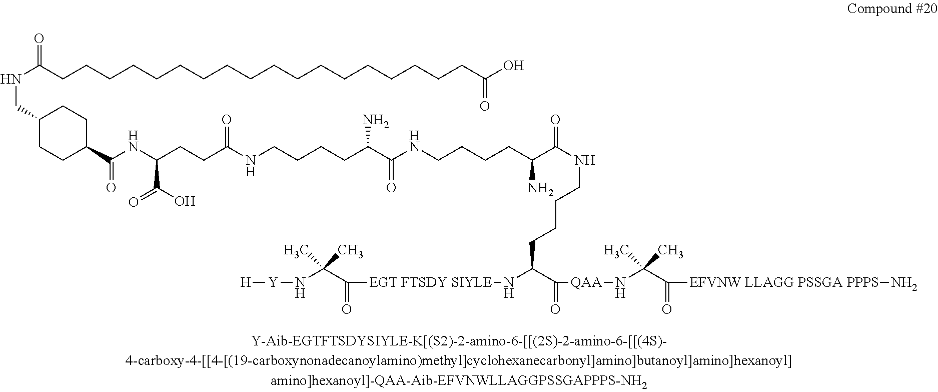

SEQ ID NO: 10 with substituent at position K16 and C-terminal amide modification.

Substituent: C20 diacid-Trx-γGlu-ELys-ELys (F)

Synthesis methods: SPPS_A; SC_B; CP_A; SX_A

Molecular weight (average) calculated: 4969.6 Da

LCMS_B: Rt=6.3 min; found [M+3H]³⁺ 1657.2, [M+4H]⁴⁺ 1243.3

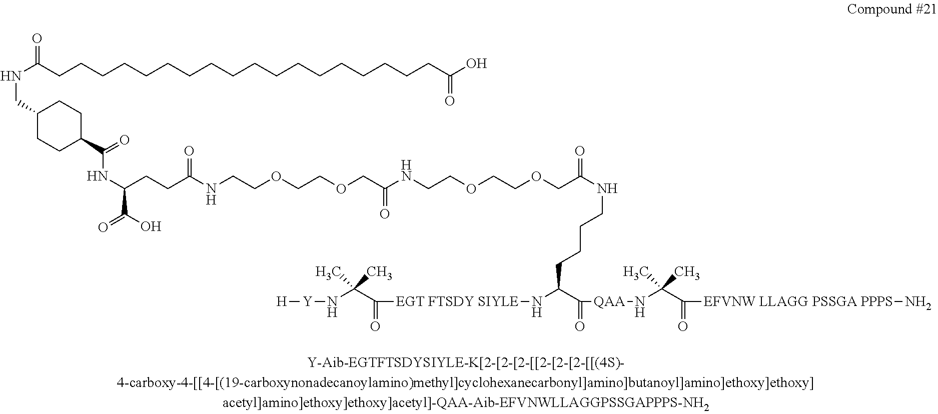

SEQ ID NO: 10 with substituent at position K16 and C-terminal amide modification.

Substituent: C20 diacid-Trx-γGlu-Ado-Ado (E)

Synthesis methods: SPPS_A; SC_B; CP_A; SX_A

Molecular weight (average) calculated: 5003.6 Da

LCMS_B: Rt=7.0 min; found [M+3H]³⁺ 1668.6, [M+4H]⁴⁺ 1251.6

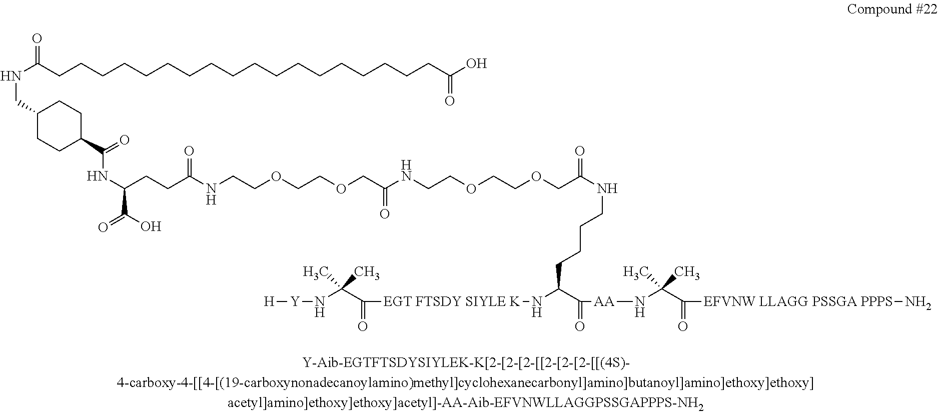

SEQ ID NO: 11 with substituent at position K17 and C-terminal amide modification.

Substituent: C20 diacid-Trx-γGlu-Ado-Ado (E)

Synthesis methods: SPPS_A; SC_B; CP_A; SX_A

Molecular weight (average) calculated: 5003.7 Da

LCMS_B: Rt=6.5 min; found [M+3H]³⁺ 1668.7, [M+4H]⁴⁺ 1251.8

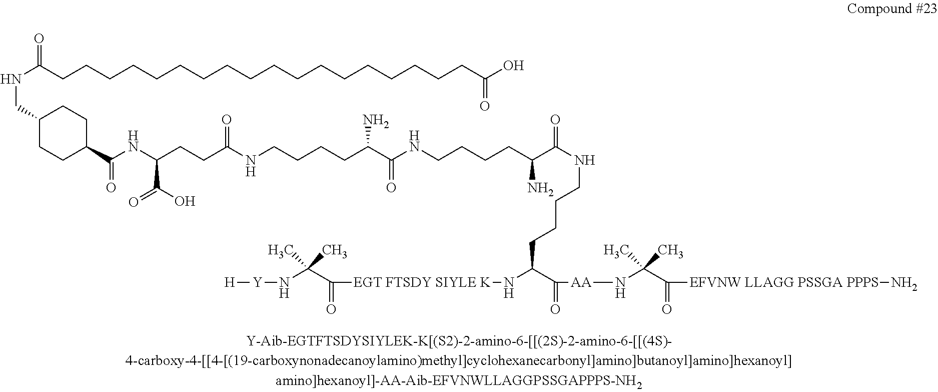

SEQ ID NO: 11 with substituent at position K17 and C-terminal amide modification.

Substituent: C20 diacid-Trx-γGlu-ELys-ELys (F)

Synthesis methods: SPPS_A; SC_A; CP_A

Molecular weight (average) calculated: 4969.7 Da

LCMS_B: Rt=6.2 min; found [M+3H]³⁺ 1657.3, [M+4H]⁴⁺ 1243.3

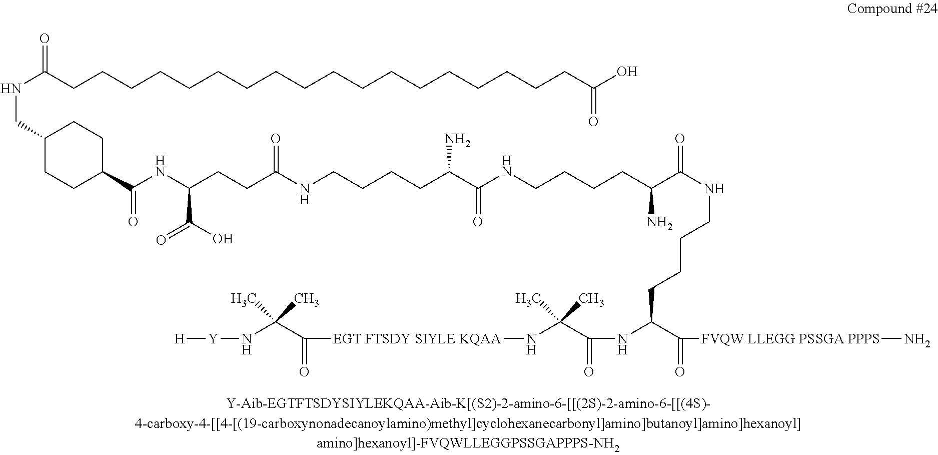

SEQ ID NO: 12 with substituent at position K21 and C-terminal amide modification.

Substituent: C20 diacid-Trx-γGlu-ELys-ELys (F)

Synthesis methods: SPPS_A; SC_A; CP_A

Molecular weight (average) calculated: 5040.8 Da

LCMS_B: Rt=6.3 min; found [M+3H]³⁺ 1680.9, [M+4H]⁴⁺ 1261.0

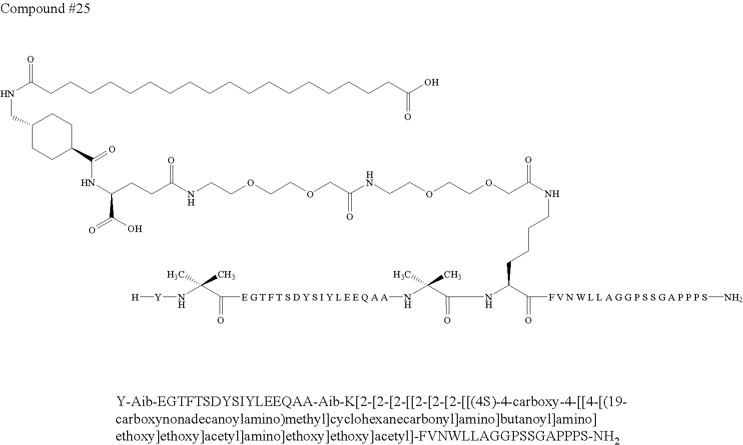

SEQ ID NO: 9 with substituent at position K21 and C-terminal amide modification.

Substituent: C20 diacid-Trx-γGlu-Ado-Ado (E)

Synthesis methods: SPPS_A; SC_B; CP_A

Molecular weight (average) calculated: 5003.6 Da

LCMS_B: Rt=7.0 min; found [M+3H]³⁺ 1668.6, [M+4H]⁴⁺ 1251.7

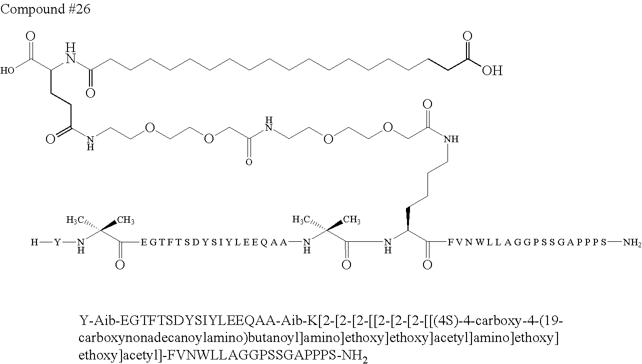

SEQ ID NO: 9 with substituent at position K21 and C-terminal amide modification.

Substituent: C20 diacid-γGlu-Ado-Ado (C)

Synthesis methods: SPPS_A; SC_B; CP_A

Molecular weight (average) calculated: 4864.4 Da

LCMS_B: Rt=6.9 min; found [M+3H]³⁺ 1622.1, [M+4H]⁴⁺ 1217.0

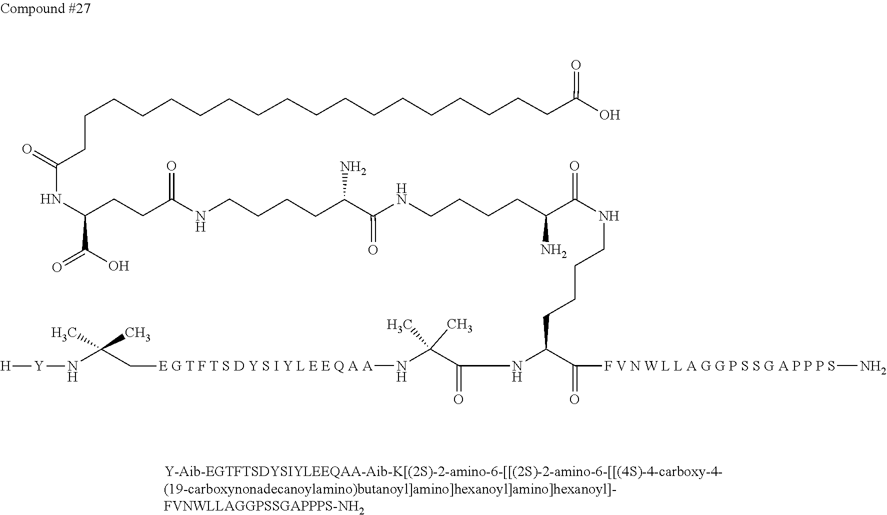

SEQ ID NO: 9 with substituent at position K21 and C-terminal amide modification.

Substituent: C20 diacid-γGlu-ELys-ELys (D)

Synthesis methods: SPPS_A; SC_B; CP_A; SX_A

Molecular weight (average) calculated: 4830.4 Da

LCMS_B: Rt=6.7 min; found [M+3H]³⁺ 1611.0, [M+4H]⁴⁺ 1206.4

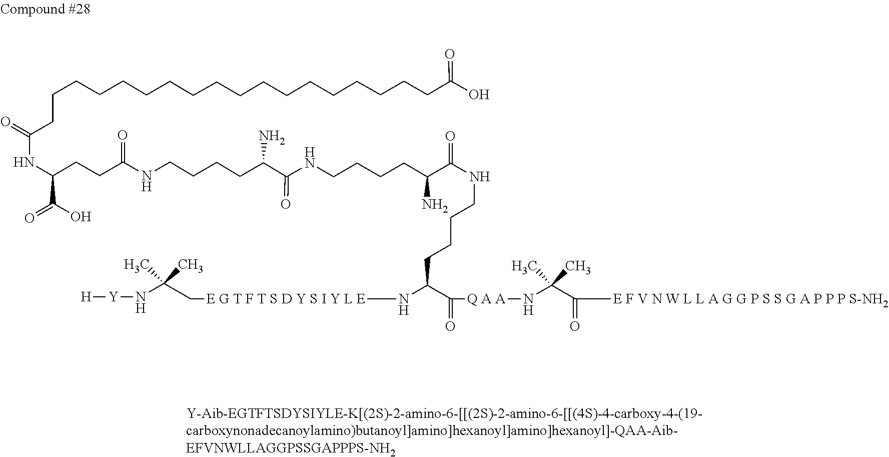

SEQ ID NO: 10 with substituent at position K16 and C-terminal amide modification.

Substituent: C20 diacid-γGlu-ELys-ELys (D)

Synthesis methods: SPPS_A; SC_B; CP_A; SX_A

Molecular weight (average) calculated: 4830.4 Da

LCMS_B: Rt=6.2 min; found [M+3H]³⁺ 1610.5, [M+4H]⁴⁺ 1208.6

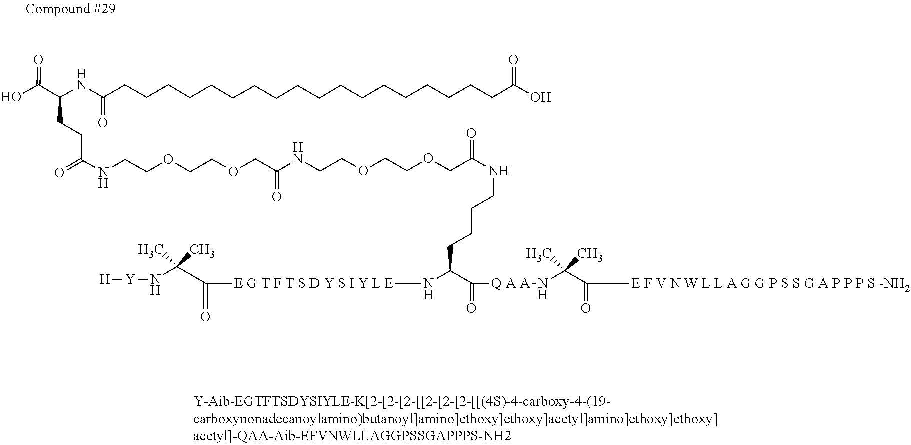

SEQ ID NO: 10 with substituent at position K16 and C-terminal amide modification.

Substituent: C20 diacid-γGlu-Ado-Ado (C)

Synthesis methods: SPPS_A; SC_A; CP_A

Molecular weight (average) calculated: 4864.4 Da

LCMS_B: Rt=7.0 min; found [M+3H]³⁺ 1622.3, [M+4H]⁴⁺ 1216.8

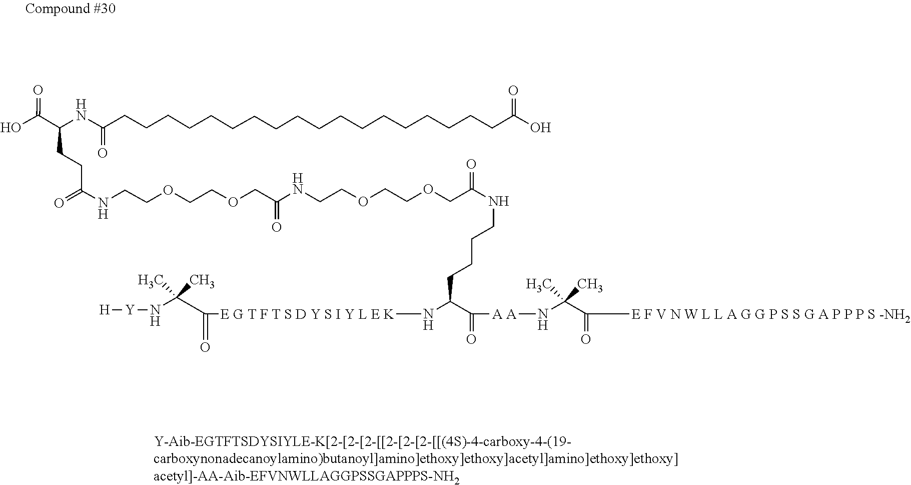

SEQ ID NO: 11 with substituent at position K17 and C-terminal amide modification Substituent: C20 diacid-γGlu-Ado-Ado (C)

Synthesis methods: SPPS_A; SC_A; CP_A

Molecular weight (average) calculated: 4864.5 Da

LCMS_B: Rt=6.3 min; found [M+3H]³⁺ 1622.1, [M+4H]⁴⁺ 1217.1

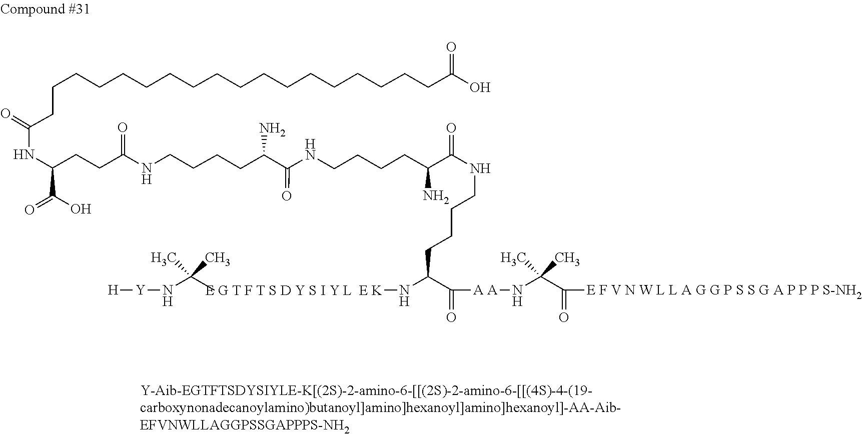

SEQ ID NO: 11 with substituent at position K17 and C-terminal amide modification Substituent: C20 diacid-γGlu-ELys-ELys (D)

Synthesis methods: SPPS_A; SC_A; CP_A

Molecular weight (average) calculated: 4830.5 Da

LCMS_B: Rt=6.0 min; found [M+3H]³⁺ 1611.0, [M+4H]⁴⁺ 1208.7

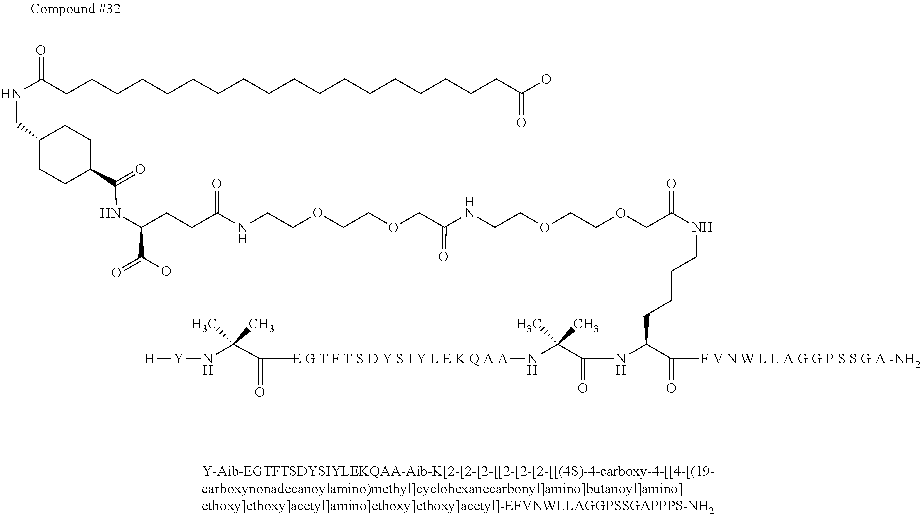

SEQ ID NO: 8 with substituent at position K21 and C-terminal amide modification Substituent: C20 diacid-Trx-γGlu-Ado-Ado (E)

Synthesis methods: SPPS_A; SC_B; CP_A

Molecular weight (average) calculated: 4624.2 Da

LCMS_B: Rt=6.6 min; found [M+3H]³⁺ 1542.2, [M+4H]⁴⁺ 1156.9

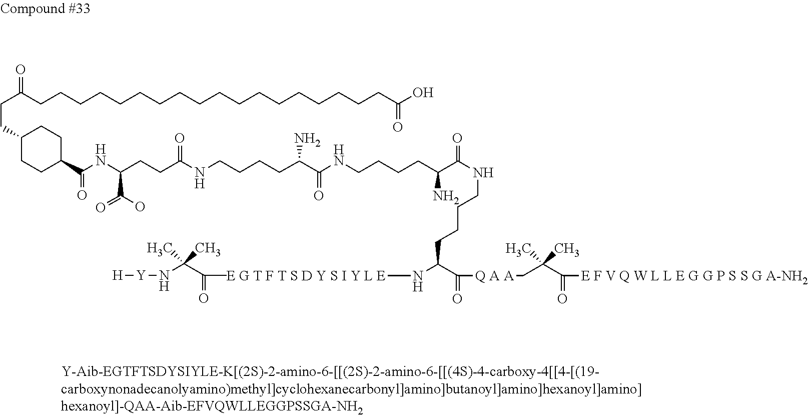

SEQ ID NO: 13 with substituent at position K16 and C-terminal amide modification Substituent: C20 diacid-Trx-γGlu-ELys-ELys (F)

Synthesis methods: SPPS_A; SC_B; CP_A

Molecular weight (average) calculated: 4663.3 Da

LCMS_B: Rt=6.4 min; found [M+3H]³⁺ 1555.1, [M+4H]⁴⁺ 1166.8

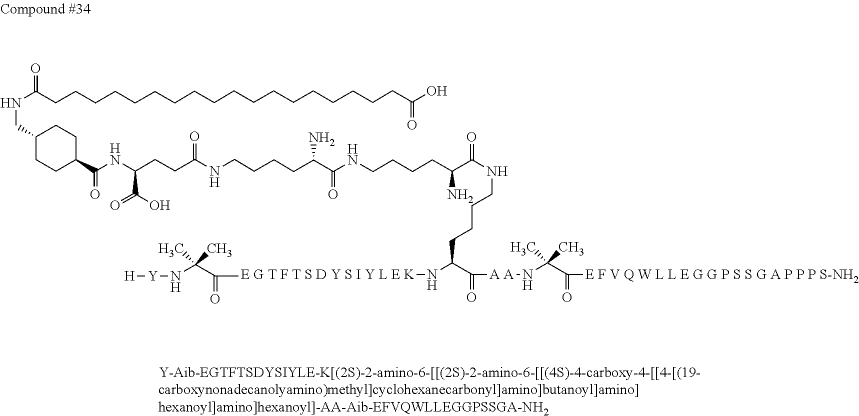

SEQ ID NO: 14 with substituent at position K17 and C-terminal amide modification Substituent: C20 diacid-Trx-γGlu-ELys-ELys (F)

Synthesis methods: SPPS_A; SC_B; CP_A

Molecular weight (average) calculated: 5041.7 Da

LCMS_B: Rt=6.3 min; found [M+3H]³⁺ 1681.4, [M+4H]⁴⁺ 1261.3

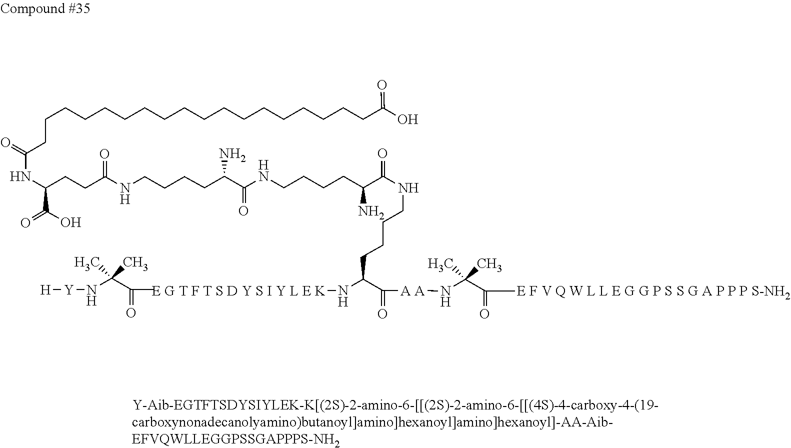

SEQ ID NO: 14 with substituent at position K17 and C-terminal amide modification Substituent: C20 diacid-γGlu-ELys-ELys (D)

Synthesis methods: SPPS_A; SC_B; CP_A; SX_A

Molecular weight (average) calculated: 4902.6 Da

LCMS_B: Rt=6.2 min; found [M+3H]³⁺ 1634.9, [M+4H]⁴⁺ 1226.6

### Example 2: In Vitro Functional Potency (CRE Luciferase; Whole Cells)

The purpose of this example is to test the functional activity, or potency, of the compounds in vitro at the human and mouse GLP-1 and GIP receptors, as well as at the human glucagon receptor. The in vitro functional potency is the measure of target receptor activation in a whole cell assay. The potencies of derivatives of Example 1 were determined as described below. Human GLP-1(7-37) (identical to mouse GLP-1(7-37)), human GIP, mouse GIP, and human glucagon were included in appropriate assays for comparison.

### Principle

In vitro functional potency was determined by measuring the response of the target receptor in a reporter gene assay in individual cell lines. The assay was performed in stably transfected BHK cell lines that expresses one of the following G-protein coupled receptors: human GLP-1 receptor, human GIP receptor, mouse GLP-1 receptor, mouse GIP receptor, or human glucagon receptor; and where each cell line contains the DNA for the cAMP response element (CRE) coupled to a promoter and the gene for firefly luciferase (CRE luciferase). When the respective receptor is activated, it results in the production of cAMP, which in turn results in expression of the luciferase protein. When assay incubation is completed, luciferase substrate (luciferin) is added resulting in the enzymatic conversion of luciferin to oxyluciferin and producing bioluminescence. The luminescence is measured as the readout for the assay.

### Cell Culture and Preparation

The cells lines used in these assays were BHK cells with BHKTS13 as a parent cell line. The cell lines were derived from a clone containing the CRE luciferase element and were established by further transfection with the respective receptor to obtain the relevant cell line. The following cell lines were used:

| | human | mouse |
|---|---|---|
| GLP-1 receptor assay | BHK CRE luc2P hGLP-1R | BHK CRE luc2P |
| | | mGLP-1R |
| GIP receptor assay | BHK CRE luc2P hGIPR | BHK CRE luc2P |
| | | mGIPR |
| Glucagon receptor assay | BHK CRE luc2P hGCGR | — |

The cells were cultured at 37° C. with 5% CO₂ in Cell Culture Medium. They were aliquoted and stored in liquid nitrogen. The cells were kept in continuous culture and were seeded out the day before each assay.

### Materials

The following chemicals were used in the assay: Pluronic F-68 10% (Gibco 2404), human serum albumin (HSA; Sigma A9511), 10% fetal bovine serum (FBS; Invitrogen 16140-071), chicken egg white ovalbumin (Sigma A5503), DMEM w/o phenol red (Gibco 21063-029), DMEM (Gibco 12430-054), 1 M Hepes (Gibco 15630), Glutamax 100×(Gibco 35050), G418 (Invitrogen 10131-027), hygromycin (Invitrogen 10687-010), and steadylite plus (PerkinElmer 6016757).

### Buffers

GLP-1R and GcgR Cell Culture Medium consisted of DMEM medium with 10% FBS, 500 μg/mL G418, and 300 μg/mL hygromycin. GIPR Cell Culture Medium consisted of DMEM medium with 10% FBS, 400 μg/mL G418, and 300 μg/mL hygromycin. Assay Buffer consisted of DMEM w/o phenol red, 10 mM Hepes, 1×Glutamax, 1% ovalbumin, and 0.1% Pluronic F-68 with the addition of HSA at twice the final assay concentration. The Assay Buffer was mixed 1:1 with an equal volume of the test compound in Assay Buffer to give the final assay concentration of HSA.

### Procedure

1) Cells were plated at 5000 cells/well and incubated overnight in the assay plate.
2) Cells were washed once in DPBS.
3) Stocks of the test compounds and reference compounds in concentrations ranging from 100-300 μM were diluted 1:150 in Assay Buffer. Compounds were then diluted 1:10 in column 1 of a 96 deep well dilution plate and then carried across the row creating a 3.5 fold, 12 point dilution curve.
4) Assay Buffer (50 μl aliquot) with or without HSA was added to each well in the assay plate.
5) A 50 μl aliquot of compound or blank was transferred from the dilution plate to the assay plate containing the Assay Buffer with or without HSA.
6) The assay plate was incubated for 3 h in a 5% CO₂ incubator at 37° C. 7) The cells were washed once with DPBS.
8) A 100 μl aliquot of DPBS was added to each well of the assay plate.
9) A 100 μl aliquot of steadylite plus reagent (light sensitive) was added to each well of the assay plate.
10) Each assay plate was covered with aluminum foil to protect it from light and shaken at 250 RPM for 30 min at room temperature.
11) Each assay plate was read in a microtiter plate reader.

### Calculations and Results

The data from the microtiter plate reader was first regressed in an Excel in order to calculate the x-axis, log scale concentrations based on the individual test compound's stock concentration and the dilutions of the assay. This data was then transferred to GraphPad Prism software for graphing and statistical analysis. The software performs a non-linear regression (log(agonist) vs response). EC₅₀ values which were calculated by the software and reported in pM are shown in Tables 1 and 2 below. A minimum of two replicates was measured for each sample. The reported values are averages of the replicates.

TABLE 1
Functional potencies at human GLP-1R and
GIPR in the presence of 0% and 1% HSA.

| | hGLP-1R, CRE | hGLP-1R, CRE | hGIPR, CRE | hGIPR, CRE |
|---|---|---|---|---|
| Compound | Luc 0% HSA | Luc 1% HSA | Luc 0% HSA | Luc 1% HSA |
| No. | EC₅₀ (pM) | EC₅₀ (pM) | EC₅₀ (pM) | EC₅₀ (pM) |
| hGLP-1(7-37) | 3.8 | 2.5 | nd | nd |
| hGIP | nd | nd | 8.5 | 3.8 |
| 1. | 2.8 | 6.5 | 4.6 | 5.8 |
| 2. | 67.5 | 438.5 | 27.8 | 550.2 |
| 3. | 13.4 | 606.0 | 19.2 | 513.8 |
| 4. | 10.6 | 231.5 | 27.3 | 217.3 |
| 5. | 9.2 | 161.1 | 11.3 | 212.1 |
| 6. | 5.9 | 244.1 | 8.1 | 101.2 |
| 7. | 55.7 | 1730.0 | 3.3 | 49.3 |
| 8. | 29.9 | 699.0 | 4.7 | 34.1 |
| 9. | 6.7 | 109.7 | 11.6 | 134.7 |
| 10. | 18.2 | 297.9 | 22.1 | 286.9 |
| 11. | 393.8 | nd | 4.1 | nd |
| 12. | 2.9 | nd | >10000 | nd |
| 13. | 12.4 | 103.4 | 14.1 | 71.4 |
| 14. | 11.4 | 377.3 | 15.8 | 98.1 |
| 15. | 6.1 | 127.7 | 3.8 | 73.6 |
| 16. | 6.1 | 201.6 | 4.5 | 141.4 |
| 17. | 4.1 | 229.2 | 3.2 | 223.2 |
| 18. | 3.3 | 89.9 | 3.3 | 85.3 |
| 19. | 4.6 | 297.1 | 4.8 | 236.9 |
| 20. | 3.3 | 260.6 | 2.3 | 125.8 |
| 21. | 3.0 | 465.3 | 2.3 | 182.0 |
| 22. | 3.3 | 1134.1 | 3.9 | 626.9 |
| 23. | 4.0 | 177.9 | 3.4 | 175.1 |
| 24. | 4.7 | 114.3 | 4.7 | 132.5 |
| 25. | 4.8 | 602.1 | 4.5 | 358.2 |
| 26. | 4.7 | 369.7 | 5.8 | 420.3 |
| 27. | 3.3 | 195.5 | 5.7 | 328.9 |
| 28. | 5.0 | 356.3 | 2.9 | 139.4 |
| 29. | 6.2 | 429.3 | 2.8 | 231.7 |
| 30. | 8.4 | 352.8 | 8.8 | 315.2 |
| 31. | 1.9 | 54.3 | 2.7 | 90.3 |
| 32. | 11.7 | 221.1 | 5.0 | 168.8 |
| 33. | 5.9 | 741.4 | 5.1 | 358.5 |
| 34. | 3.2 | 379.1 | 3.1 | 180.8 |
| 35. | 2.2 | 168.9 | 3.7 | 155.4 |

nd = not determined.

TABLE 2
Functional potencies at mouse GLP-1R and
GIPR in the absence of plasma proteins.

| | mGLP-1R, | mGIPR, |
|---|---|---|
| | CRE Luc | CRE Luc |
| Compound | EC₅₀ (pM) | EC₅₀ (pM) |
| mGLP-1(7-37) | 3.5 | nd |
| mGIP | nd | 35.4 |
| 1. | 2.0 | 8.0 |
| 2. | nd | nd |
| 3. | nd | nd |
| 4. | 3.9 | 1552.0 |
| 5. | 2.5 | 522.6 |
| 6. | 2.3 | 1267.5 |
| 7. | 17.4 | 42.5 |
| 8. | 17.0 | 23.3 |
| 9. | 2.5 | 68.0 |
| 10. | 2.1 | 258.6 |
| 11. | 280.8 | 13.7 |
| 12. | 2.2 | >10000.0 |
| 13. | 5.4 | 57.0 |
| 14. | 6.0 | 18.6 |
| 15. | 1.7 | 27.3 |
| 16. | 3.5 | 16.9 |
| 17. | 2.3 | 21.9 |
| 18. | 2.0 | 24.3 |
| 19. | 5.5 | 133.5 |
| 20. | 2.6 | 12.0 |
| 21. | 2.6 | 36.6 |
| 22. | 2.1 | 123.6 |
| 23. | 3.7 | 17.5 |
| 24. | 9.9 | 40.5 |
| 25. | 2.9 | 339.7 |
| 26. | 2.8 | 776.5 |
| 27. | 4.2 | 544.6 |
| 28. | 2.3 | 26.0 |
| 29. | 1.8 | 51.0 |
| 30. | 1.9 | 201.2 |
| 31. | 1.3 | 13.4 |
| 32. | 2.5 | 80.2 |
| 33. | 4.0 | 31.1 |
| 34. | 1.8 | 19.5 |
| 35. | 1.3 | 21.3 |

nd = not determined.

The compounds of the present invention display potent functional activation of the human GLP-1R, human GIPR, mouse GLP-1R, and mouse GIP receptors under the given conditions. Alterations that allow for potency to be maintained between mouse-specific and human-specific receptors give more confidence in translation of in vivo results from mouse to human. Furthermore, the compounds display minimal to no measurable functional activation of the human glucagon receptor, as shown in Table 3 below.

TABLE 3
Potencies at human glucagon receptor
in the absence of plasma proteins.

| | hGcgR, |
|---|---|
| | CRE Luc |
| Compound | EC₅₀ (pM) |
| hGlucagon | 17.0 |
| 1. | 2855.5 |
| 9. | >10000.0 |
| 13. | >10000.0 |
| 14. | >10000.0 |
| 16. | >10000.0 |
| 17. | >10000.0 |
| 19. | >10000.0 |
| 20. | >10000.0 |
| 21. | >10000.0 |
| 22. | >10000.0 |
| 23. | >10000.0 |
| 24. | >10000.0 |
| 28. | >10000.0 |
| 29. | >10000.0 |
| 31. | 9601.0 |

The compounds of the present invention display minimal to no measurable functional activation of the human glucagon receptor, thus providing selective co-agonists of GLP-1R and GIPR.

### Example 3: Pharmacokinetic Study in Minipigs

The purpose of this example is to determine the half-life in vivo of the derivatives of the present invention after i.v. administration to minipigs, i.e. the prolongation of their time in the body and thereby their time of action. This is done in a pharmacokinetic (PK) study, where the terminal half-life of the derivative in question is determined. By terminal half-life is generally meant the period of time it takes to halve a certain plasma concentration, measured after the initial distribution phase.

### Study

Female Göttingen minipigs were obtained from Ellegaard Göttingen Minipigs (Dalmose, Denmark) approximately 7-14 months of age and weighing from approximately 16-35 kg were used in the studies. The minipigs were housed individually and fed restrictedly once daily with SDS minipig diet (Special Diets Services, Essex, UK).

After at 3 weeks of acclimatisation two permanent central venous catheters were implanted in vena cava caudalis in each animal. The animals were allowed 1 week recovery after the surgery, and were then used for repeated pharmacokinetic studies with a suitable wash-out period between successive derivative dosing.

The animals were fasted for approximately 18 hours before dosing and from 0 to 4 hours after dosing, but had adlibitum access to water during the whole period.

The sodium salts of compounds of Examples 1 were dissolved to a concentration of 20-40 nmol/mL in a buffer containing 0.025% polysorbate 20, 10 mM sodium phosphate, 250 mM glycerol, pH 7.4. Intravenous injections (the volume corresponding to usually 1.5-2 nmol/kg, for example 0.1 mL/kg) of the compounds were given through one catheter, and blood was sampled at predefined time points for up to 14 days post dosing (preferably through the other catheter). Blood samples (for example 0.8 mL) were collected in 8 mM EDTA buffer and then centrifuged at 4° C. and 1942 g for 10 minutes.

### Sampling and Analysis

Plasma was pipetted into Micronic tubes on dry ice and kept at −20° C. until analysed for plasma concentration of the compounds using ELISA, or a similar antibody-based assay, or LCMS. Individual plasma concentration-time profiles were analysed by a non-compartmental model in Phoenix WinNonLin ver. 6.4. (Pharsight Inc., Mountain View, CA, USA), and the resulting terminal half-lives (harmonic mean) determined.

### Results

TABLE 4
Terminal half-life as measured after i.v. administration to minipigs

| Compound No. | t_{1/2} (h) |
|---|---|
| 19 | 112 |
| 20 | 88 |
| 21 | 62 |
| 22 | 95 |
| 27 | 88 |
| 28 | 111 |
| 35 | 90 |

The tested compounds of the present invention have very long half-lives as compared to the half-lives of hGLP-1 and hGIP measured in man to be approximately 2-4 min and 5-7 min, respectively (Meier et al., Diabetes, 2004, 53(3): 654-662). The measured half-lives in minipigs predict half-lives in humans sufficient for at least once-weekly administration via liquid injection.

### Example 4: Pharmacodynamic Study Studies in Diet-Induced Obese (DIO) Mice

The purpose of this example is to assess the in vivo effect of select compounds on pharmacodynamic parameters in diet-induced obese (DIO) mice. The animals were treated once daily via subcutaneous injection with a liquid formulation of the test compound to assess effects on body weight, foot intake, and glucose tolerance. For comparison, the known GLP-1R/GIPR co-agonist tirzepatide and a surrogate of the GLP-1R agonist semaglutide were used as references. The semaglutide surrogate has the same pharmacological properties of semaglutide but a slightly modified structure in which the γGlu element of the substituent has been changed from the L-isomer to the D-isomer. The semaglutide surrogate and tirzepatide were synthesised using methods known in the art, e.g. as described by methods of Example 1 above, WO 2006/097537 Example 4, or WO 2016/111971 Example 1.

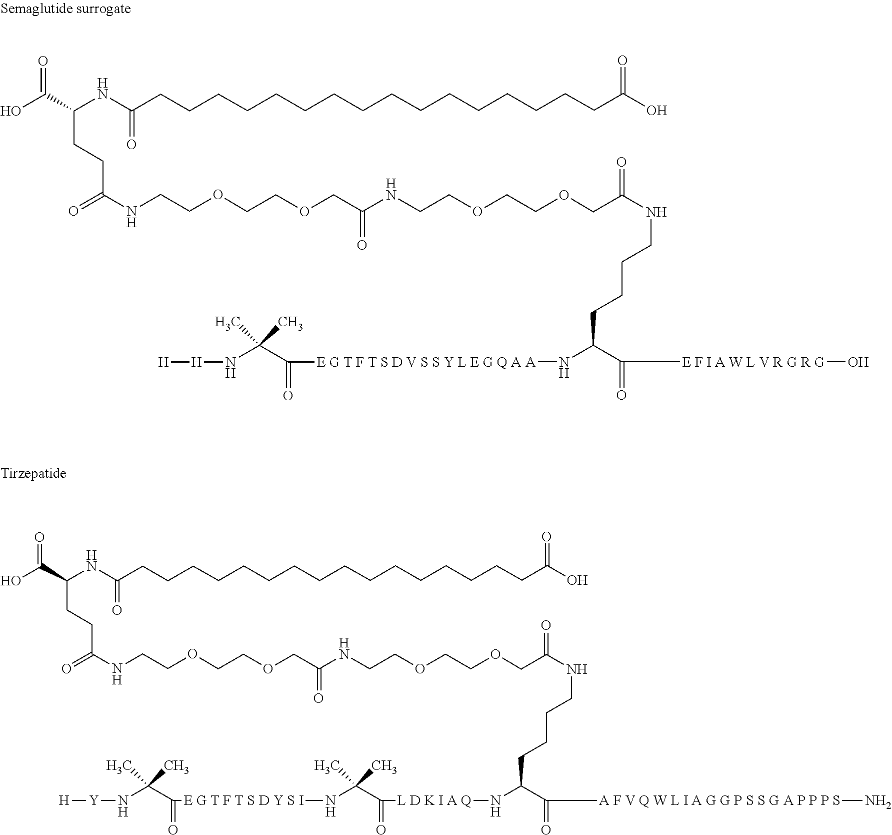

### Animals and Diet

C57BL/6J male mice were purchased from Jackson Laboratories at approximately 8 weeks of age. Mice were group housed and fed a high-fat, high-sugar diet from Research Diets (D12331). Mice were maintained on this diet for 12 weeks prior to initializing the pharmacology studies. Mice exceeding a measured body weight of 50 grams were considered diet-induced obese (DIO) and included in pharmacology studies. Mice were exposed to a controlled 12 h:12 h light:dark cycle at ambient room temperature (22° C.) with ab libitum access to food and water. Studies were approved by and performed according to the guidelines of the Institutional Animal Care and Use Committee of the University of Cincinnati.

### Dosing and Formulation

Animals were dosed once daily, subcutaneously with either vehicle or test compound. All injections occurred during the middle of the light phase at a fixed volume of 2-5 microliters per gram body weight.

All compounds in the study were formulated in the following buffer: 50 mM phosphate; 70 mM sodium chloride; 0.05% Tween-80, pH 7.4. Dosing solutions were formulated in glass vials and stored at 2-8° C. Dosing solutions were brought to room temperature before dosing and returned to 2-8° C. after dosing.

DIO mice were distributed into groups (n=8 per group) such that statistical variations in the mean and standard deviations of fat mass and body weight were minimized between groups. The animals were grouped to receive treatment as follows: Vehicle, tirzepatide, semaglutide surrogate or a GLP-1/GIP receptor co-agonists as described herein, where vehicle is 50 mM phosphate, 70 mM sodium chloride; 0.05% Tween-80, pH 7.4. The test compounds were dissolved in the vehicle, to stock concentrations of 100 μM, then diluted 50-200 fold in the vehicle to achieve the desired dosing solution concentrations. Animals were dosed subcutaneously once daily in the morning for each day of treatment with dosing solution at a volume of 2-5 μL per gram of body weight as necessary to achieve the desired dose (eg 0.3 nmol/kg, 1.0 nmol/kg, or 3.0 nmol/kg).

### Body Weight and Food Intake

Body weight (BW) and food intake were measured immediately prior to dosing each day. The percent change in body was calculated individually for each mouse based on initial body weight prior to the first injection.

### IPGTT (Intraperitoneal Glucose Tolerance Test)

On the day of the glucose tolerance test, animals were fasted for 4 h. Food was removed and animals were transferred to fresh cages. Animals had access to water but not to food. Tail blood glucose levels were measured, and mice were injected (t=0) with an intra-peritoneal (i.p.) glucose load of 2 g/kg (200 mg/ml glucose solution, dose volume 10 ml/kg). Tail blood glucose levels were measured at times 0, 15, 30, 60, 90, 120 minutes following the i.p. glucose load. Stratification of the animals during the IPGTT was such that for example two mice from group 1 are dosed followed by two mice from group 2, 3, 4, before the next two mice from group 1, 2, 3 etc. were handled. This was to allow for equal distribution of “time of day” throughout all groups.

### Results:

In one study, DIO mice received a daily subcutaneous dose of compound 9 or semaglutide surrogate at a dose of 0.3 nmol/kg, 1.0 nmol/kg, or 3.0 nmol/kg for 30 days. Results are shown in Table 5. Both compounds demonstrated dose-dependent response on all of food intake, body weight and glucose tolerance. Compound 9 demonstrated superior performance to semaglutide surrogate in all parameters at 1.0 nmol/kg and 3.0 nmol/kg doses, indicating the important effect of co-agonism on these outcomes.

TABLE 5
Effects on food intake, body weight and glucose tolerance in DIO mice treated
daily with compound 9 or semaglutide surrogate at indicated doses

| | Cumulative | | | Change | |
|---|---|---|---|---|---|
| | food intake | Absolute BW | | in BW | iAUC, IPGTT |
| | (grams) | (grams) | | (%) | (min*mg/dL) |
| Compound no. | Day 31 | Day 0 | Day 31 | Day 31 | Day 31 |
| Vehicle | 92.4 ± 12.1 | 66.5 ± 1.7 | 68.6 ± 2.9 | 3.8 ± 2.3 | 24928 ± 2309 |

0.3 nmol/kg

| | | | | | |
|---|---|---|---|---|---|
| 9 | 87.4 ± 5.9 | 66.5 ± 2.6 | 66.9 ± 3.2 | 3.4 ± 1.7 | 14979 ± 3423 |
| Semaglutide surrogate | 78.6 ± 3.9 | 66.8 ± 2.6 | 66.7 ± 2.2 | 0.1 ± 1.5 | 20761 ± 4931 |

1.0 nmol/kg

| | | | | | |
|---|---|---|---|---|---|
| 9 | 60.4 ± 4.5 | 70.0 ± 1.9 | 50.9 ± 2.2 | −23.9 ± 2.6 | 7714 ± 1722 |
| Semaglutide surrogate | 66.5 ± 1.6 | 67.9 ± 2.9 | 56.6 ± 2.6 | −16.8 ± 2.0 | 9535 ± 2855 |

3.0 nmol/kg

| | | | | | |
|---|---|---|---|---|---|
| 9 | 46.4 ± 6.6 | 65.8 ± 1.4 | 40.0 ± 2.4 | −39.1 ± 3.8 | 4073 ± 1764 |
| Semaglutide surrogate | 66.1 ± 5.3 | 66.0 ± 2.0 | 49.7 ± 2.6 | −24.8 ± 2.4 | 8167 ± 1824 |

Results expressed as mean ± SEM, n = 2 (food intake) or n = 4-8 (body weight, IPGTT). iAUC = baseline subtracted area under the curve.

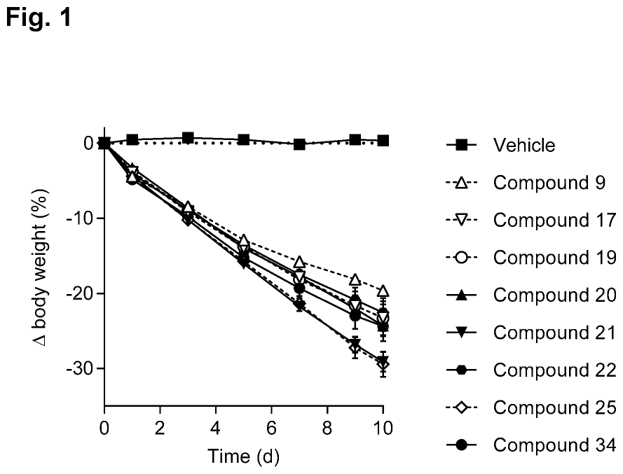

In another study, 010 mice received a daily subcutaneous dose of one of eight GLP-1/GIP receptor co-agonists at 3.0 nmol/kg for 10 days. Effects on food intake and body weight were observed. All tested co-agonists displayed a strong effect to reduce food intake and body weight compared to vehicle, as shown in FIG. 1 and Table 6 below. These results demonstrate that optimization of potency at mouse-specific receptors can result in improved efficacy in this pre-clinical model.

TABLE 6
Effects on food intake and body weight in DIO mice treated
daily with GLP-1/GIP receptor co-agonists at 3.0 nmol/kg.

| | Cumulative | | | Change |
|---|---|---|---|---|
| | food intake | Absolute BW | | in BW |
| Compound | (grams) | (grams) | | (%) |
| no. | Day 10 | Day 0 | Day 10 | Day 10 |
| Vehicle | 25.4 ± 0.9 | 63.2 ± 1.9 | 63.5 ± 2.0 | 0.3 ± 0.6 |
| 9 | 8.3 ± 1.0 | 63.8 ± 0.8 | 51.3 ± 0.9 | −19.6 ± 0.7 |
| 17 | 6.9 ± 1.3 | 63.4 ± 2.1 | 48.7 ± 2.2 | −23.2 ± 1.8 |
| 19 | 6.4 ± 1.1 | 63.7 ± 1.9 | 49.0 ± 2.4 | −23.3 ± 2.2 |
| 20 | 5.9 ± 0.3 | 64.5 ± 1.4 | 48.9 ± 1.5 | −24.3 ± 1.4 |
| 21 | 5.7 ± 0.5 | 61.9 ± 1.6 | 43.9 ± 1.5 | −29.1 ± 1.3 |
| 22 | 6.4 ± 2.6 | 63.2 ± 1.7 | 49.0 ± 2.0 | −22.6 ± 1.9 |
| 25 | 4.9 ± 1.8 | 63.0 ± 1.4 | 44.4 ± 1.2 | −29.4 ± 1.7 |
| 34 | 6.7 ± 0.2 | 63.3 ± 1.8 | 47.9 ± 2.0 | −24.4 ± 2.0 |

Results are expressed as mean ± SEM, n = 2 (food intake) or n = 8 (body weight).

Further studies using compounds 19 and 20 demonstrated dose dependent response on all of food intake, body weight and glucose tolerance in DIO mice after daily subcutaneous dosing over 14 days. Effects to reduce food intake and reduce body weight at 1.0 nmol/kg and 3.0 nmol/kg doses for both compounds surpassed Tirzepatide at equivalent doses, as shown in Table 7 below. These results demonstrate that optimization of potency at mouse-specific receptors can result in improved efficacy in this pre-clinical model.

TABLE 7
Effects on food intake, body weight and glucose tolerance in DIO mice treated daily
with compound 19, 20, or Tirzepatide at indicated doses

| | Cumulative | | | Change | |
|---|---|---|---|---|---|
| | food intake | Absolute BW | | in BW | iAUC, IPGTT |
| Compound | (grams) | (grams) | | (%) | (min*mg/dL) |
| no. | Day 14 | Day 0 | Day 14 | Day 14 | Day 15 |
| Vehicle | 38.6 ± 0.3 | 63.2 ± 1.6 | 62.6 ± 1.6 | −0.9 ± 0.2 | 21857 ± 3052 |

0.3 nmol/kg

| | | | | | |
|---|---|---|---|---|---|
| 19 | 27.0 ± 0.2 | 62.1 ± 2.0 | 54.7 ± 1.9 | −11.9 ± 0.5 | 11041 ± 1835 |
| 20 | 28.6 ± 0.2 | 63.5 ± 1.4 | 57.3 ± 1.2 | −9.8 ± 0.9 | 15599 ± 3145 |
| Tirzepatide | 32.3 ± 1.9 | 64.1 ± 1.7 | 58.2 ± 1.9 | −9.4 ± 1.2 | 13005 ± 2962 |

1.0 nmol/kg

| | | | | | |
|---|---|---|---|---|---|
| 19 | 15.9 ± 0.7 | 62.6 ± 2.2 | 45.6 ± 2.4 | −27.3 ± 2.3 | 10024 ± 1685 |
| 20 | 12.6 ± 1.5 | 63.7 ± 1.2 | 46.3 ± 1.5 | −27.4 ± 1.5 | 12117 ± 1680 |
| Tirzepatide | 18.4 ± 0.7 | 63.5 ± 1.6 | 49.8 ± 1.8 | −21.0 ± 1.2 | 10365 ± 3585 |

3.0 nmol/kg

| | | | | | |
|---|---|---|---|---|---|
| 19 | 8.6 ± 0.1 | 62.8 ± 1.8 | 39.2 ± 1.5 | −37.8 ± 0.8 | 9288 ± 2363 |
| 20 | 8.4 ± 0.3 | 63.4 ± 2.0 | 38.9 ± 1.1 | −38.6 ± 1.2 | 9191 ± 2262 |
| Tirzepatide | 16.4 ± 2.3 | 63.6 ± 2.5 | 45.1 ± 2.1 | −29.0 ± 2.4 | 8974 ± 1804 |

Results expressed as mean ± SEM, n = 2-3 (food intake) or n = 5-8 (body weight, IPGTT).

While certain features of the invention have been illustrated and described herein, many modifications, substitutions, changes, and equivalents will now occur to those of ordinary skill in the art. It is, therefore, to be understood that the appended embodiments are intended to cover all such modifications and changes as fall within the true spirit of the invention.

1. A compound comprising a peptide and a substituent; wherein the amino acid sequence of the peptide is:
(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉,
wherein
X₂ is Aib,
X₁₅ is D or E,
X₁₆ is E or K,
X₁₇ is Q or K,
X₂₀ is Aib,
X₂₁ is E or K,
X₂₄ is N or Q,
X₂₈ is A or E,
X₃₂ is S or absent,
X₃₃ is S or absent,
X₃₄ is G or absent,
X₃₅ is A or absent,
X₃₆ is P or absent,
X₃₇ is P or absent,
X₃₈ is P or absent, and
X₃₉ is S or absent;
wherein the substituent is attached to the peptide via a Lysine (K) residue in position 16, 17 or 21;
and wherein the substituent is selected from the group consisting of 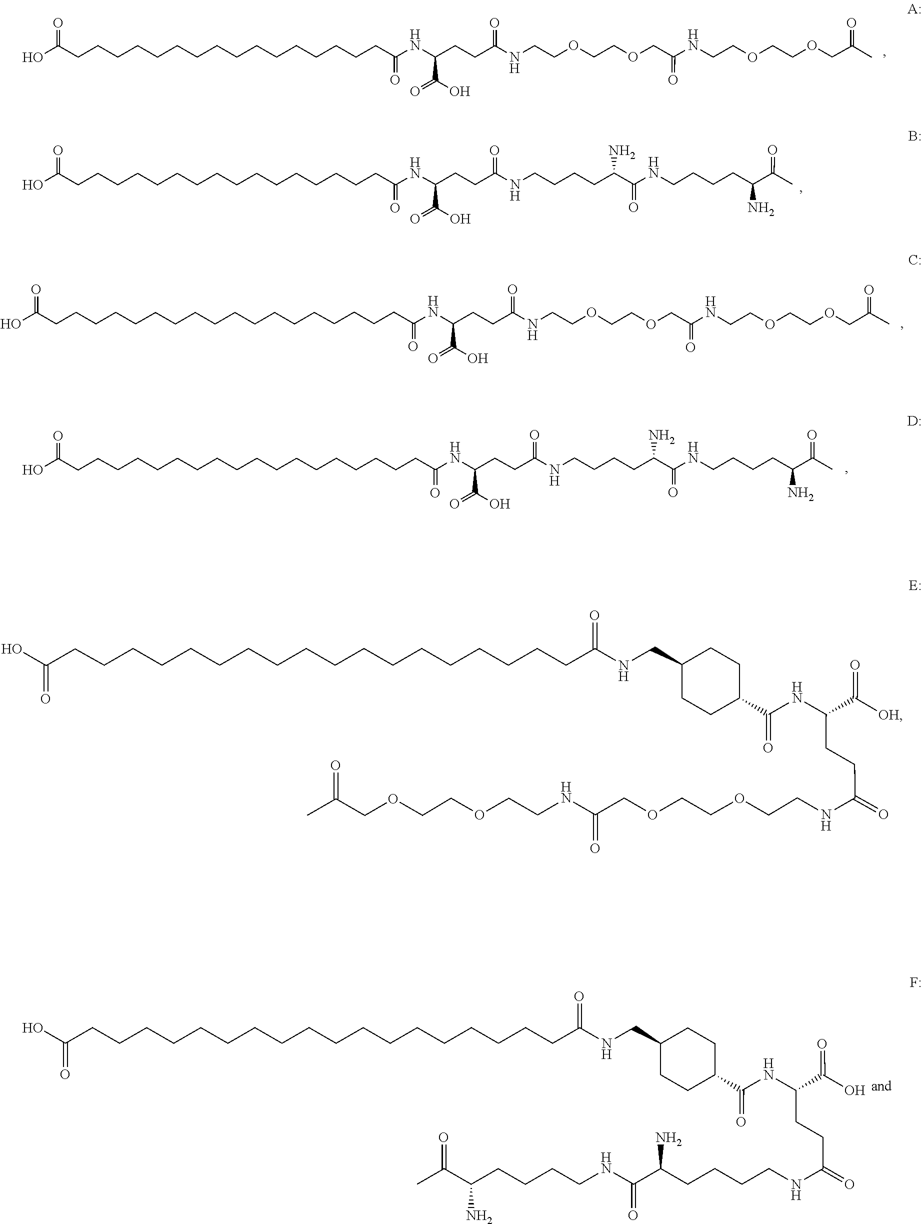 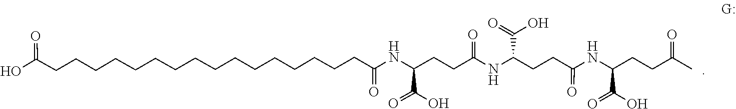 [SMILES: CC(=O)CC[C@H](NC(=O)CC[C@H](NC(=O)CC[C@H](NC(=O)CCCCCCCCCCCCCCCCC(=O)O)C(=O)O)C(=O)O)C(=O)O]
or a pharmaceutically acceptable salt hereof.

2. The compound according to claim 1, wherein X₃₆, X₃₇, X₃₈, and X₃₉ are absent.

3. The compound according to claim 1, wherein X₃₂X₃₃X₃₄X₃₅ is SSGA.

4. The compound according to claim 1, wherein the amino acid sequence of the peptide is
(SEQ ID NO.: 16)
YX₂EGTFTSDYSIYLX₁₅X₁₆X₁₇AAX₂₀X₂₁FVX₂₄WLLX₂₈GGPSSGA,
and wherein
X₂ is Aib,
X₁₅ is D or E,
X₁₆ is E or K,
X₁₇ is Q or K,
X₂₀ is Aib,
X₂₁ is E or K,
X₂₄ is N or Q, and
X₂₈ is A or E.

5. The compound according to claim 1, wherein X₁₆X₁₇AAX₂₀X₂₁ is selected from the group consisting of: KQAAAibE, KKAAAibE, KQAAAibK and EQAAAibK.

6. The compound according to claim 1, wherein the amino acid sequence of the peptide is selected from the group consisting of SEQ ID NO.: 2, SEQ ID NO.: 3, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, and SEQ ID NO.: 14.

7. The compound according to claim 6, wherein the amino acid sequence is SEQ ID NO.: 10.

8. The compound according to claim 1, wherein the substituent is attached at position 16.

9. (canceled)

10. The compound according to claim 1, wherein the compound is selected from the group consisting of:

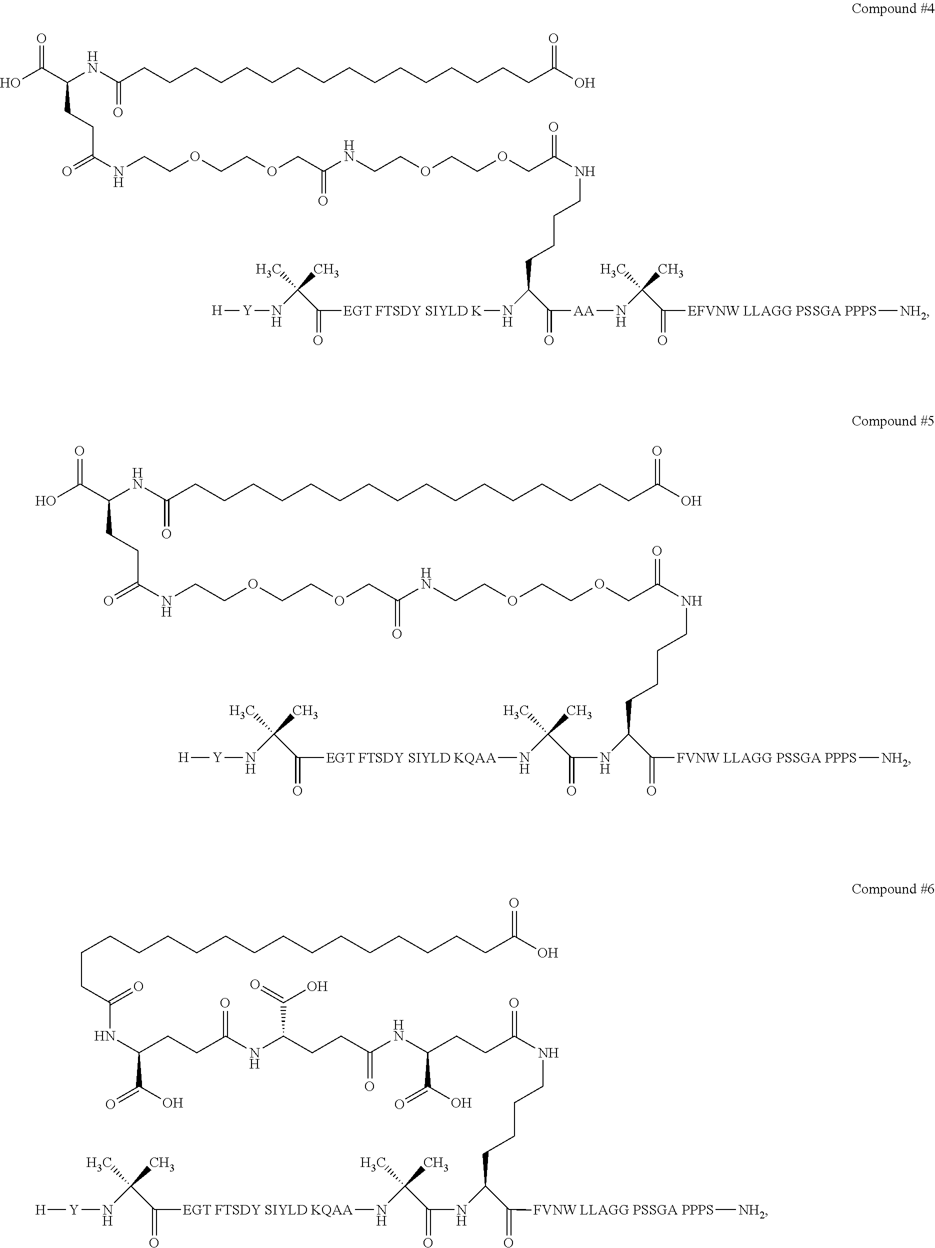

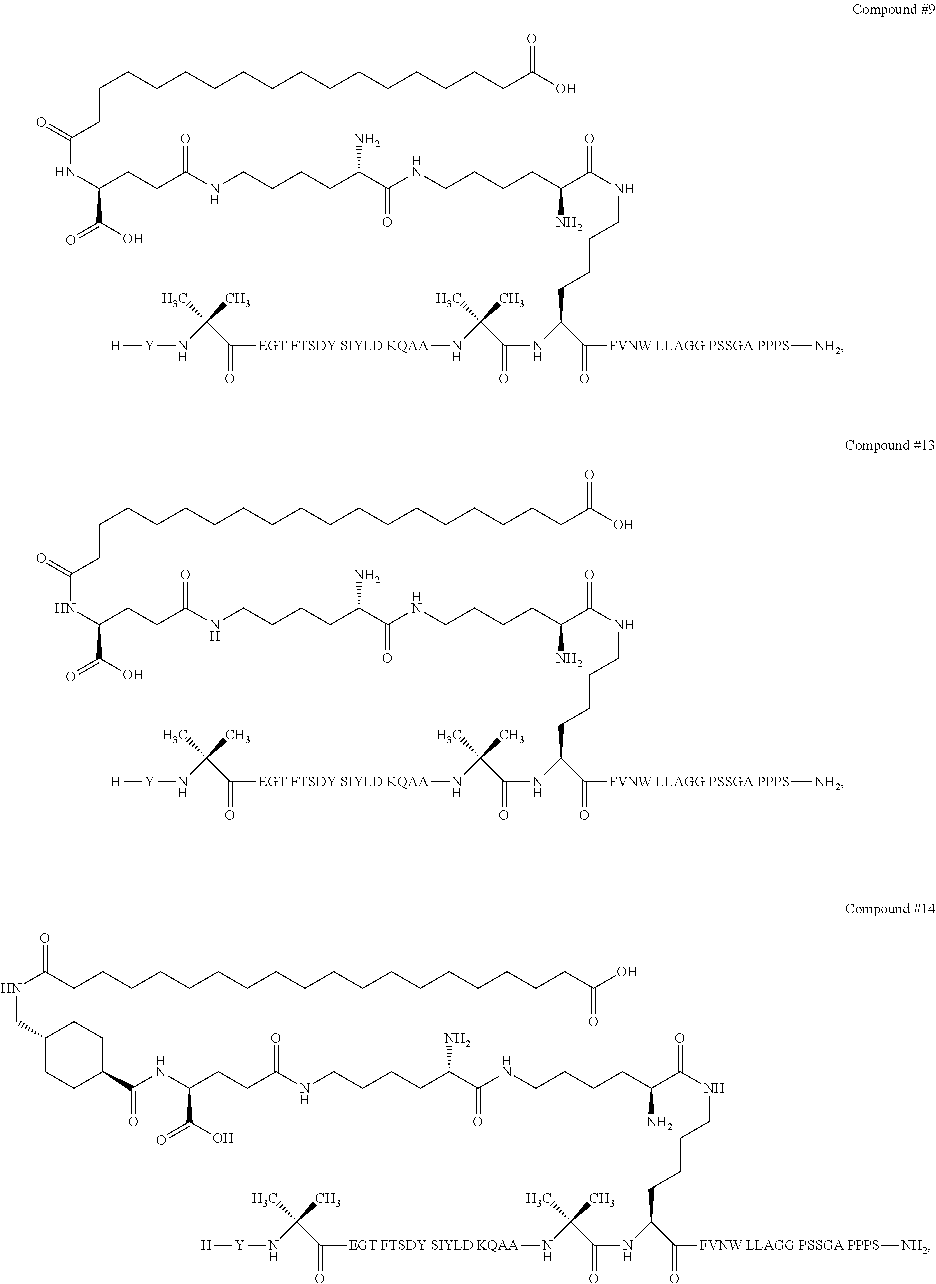

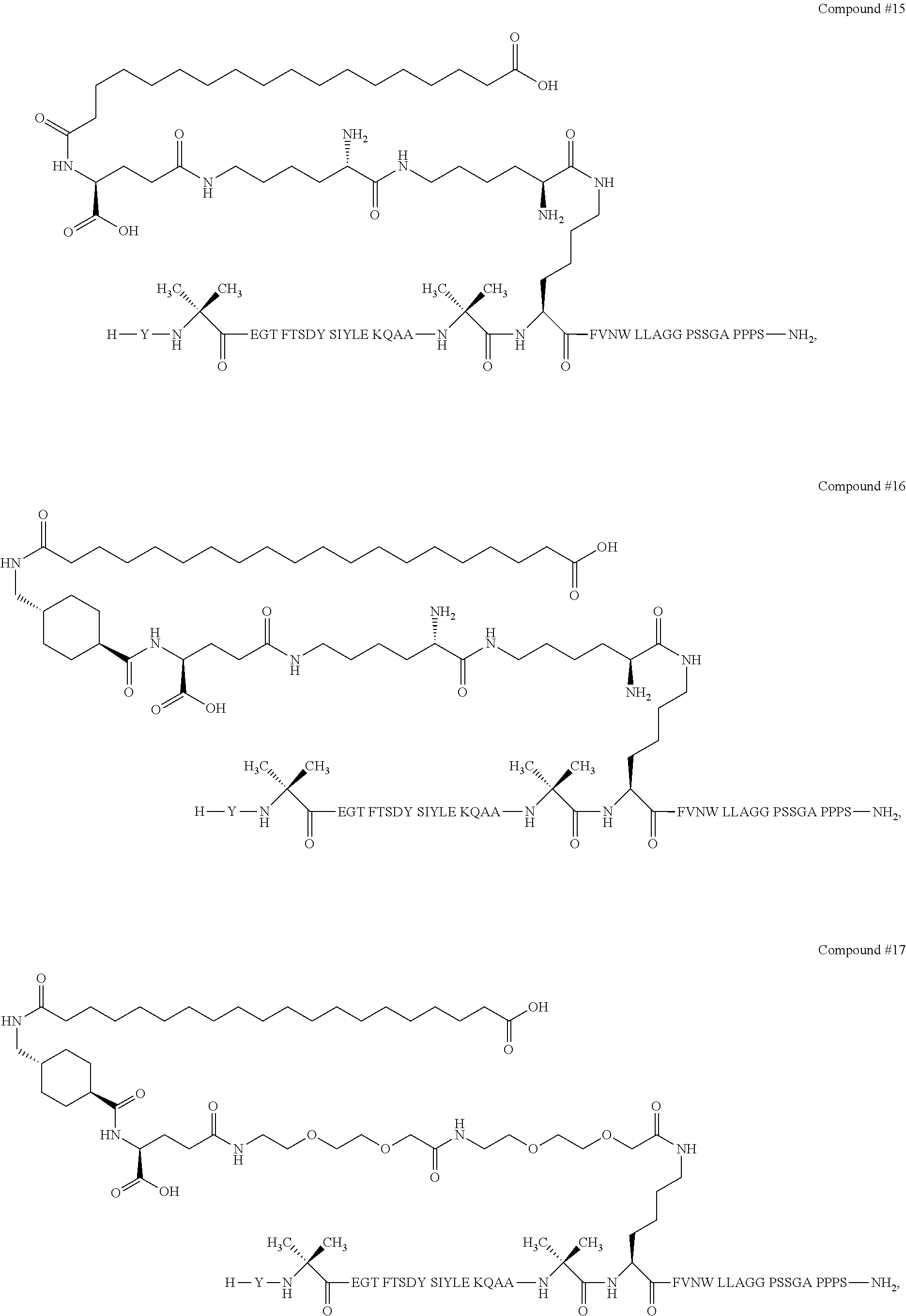

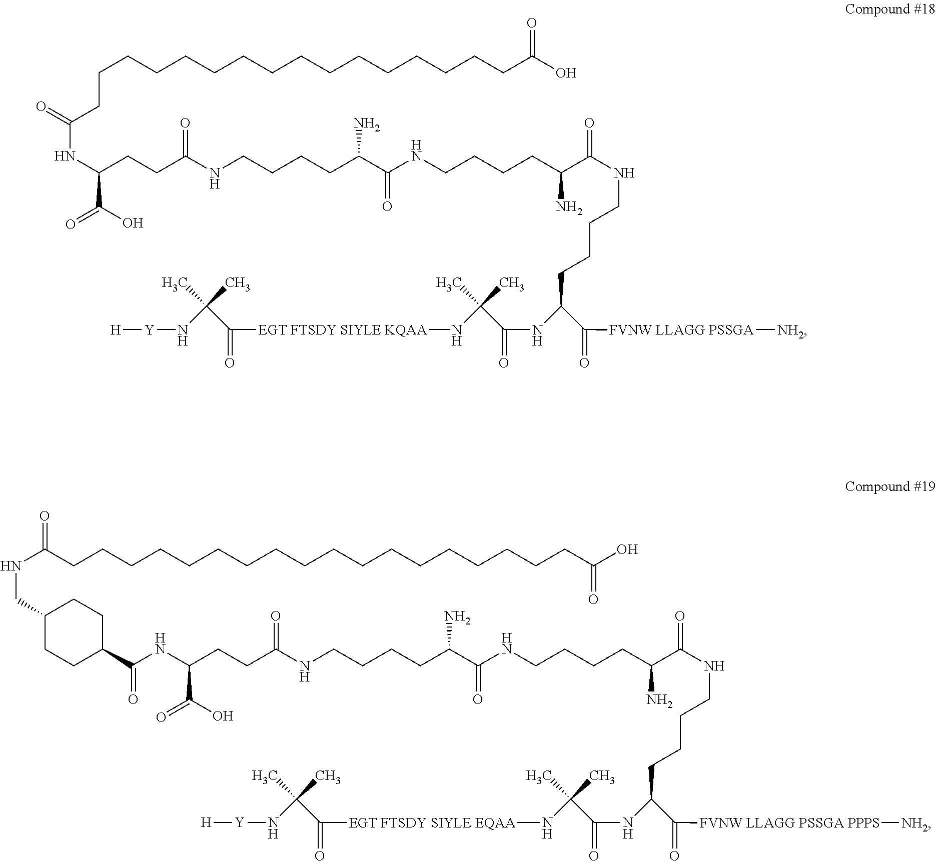

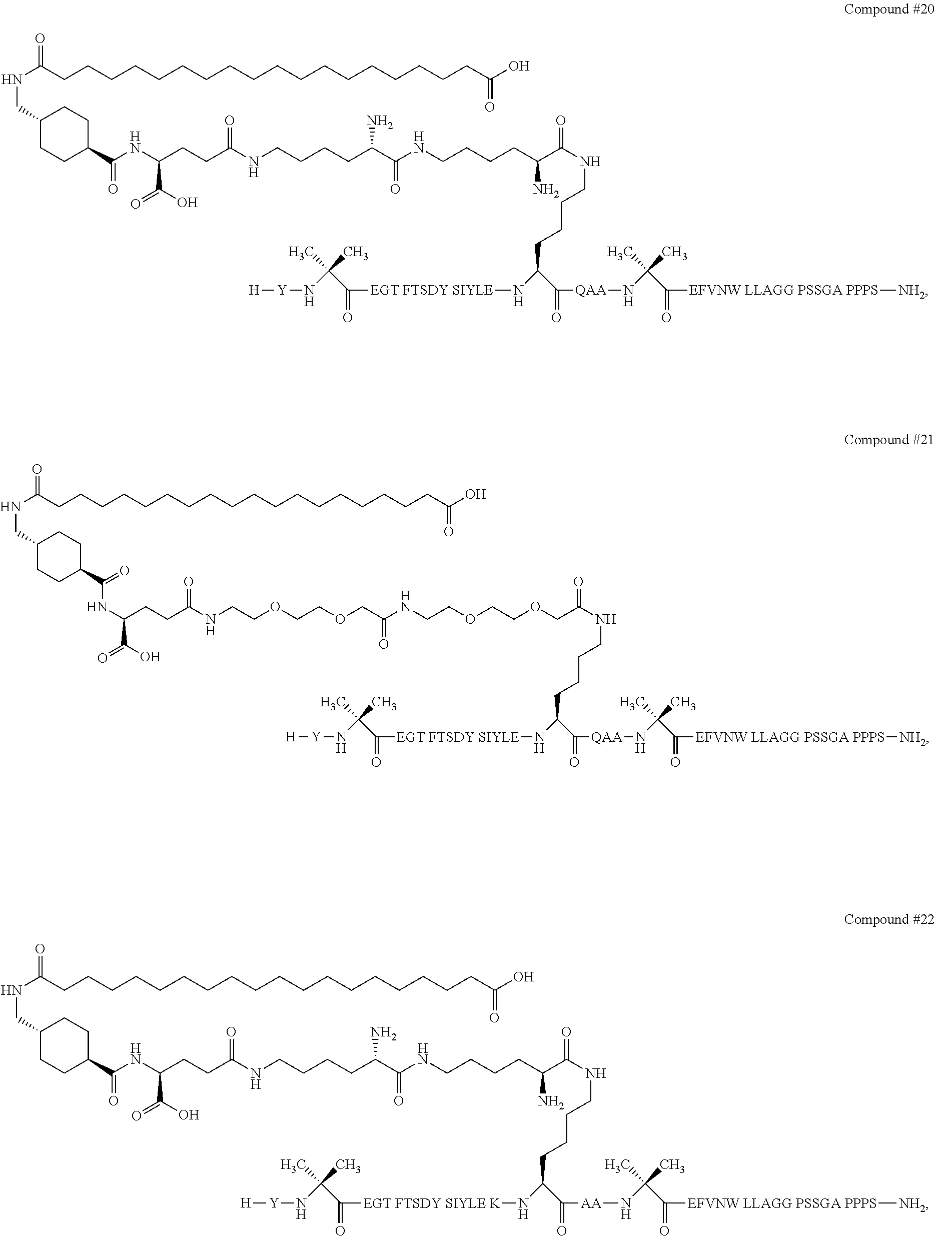

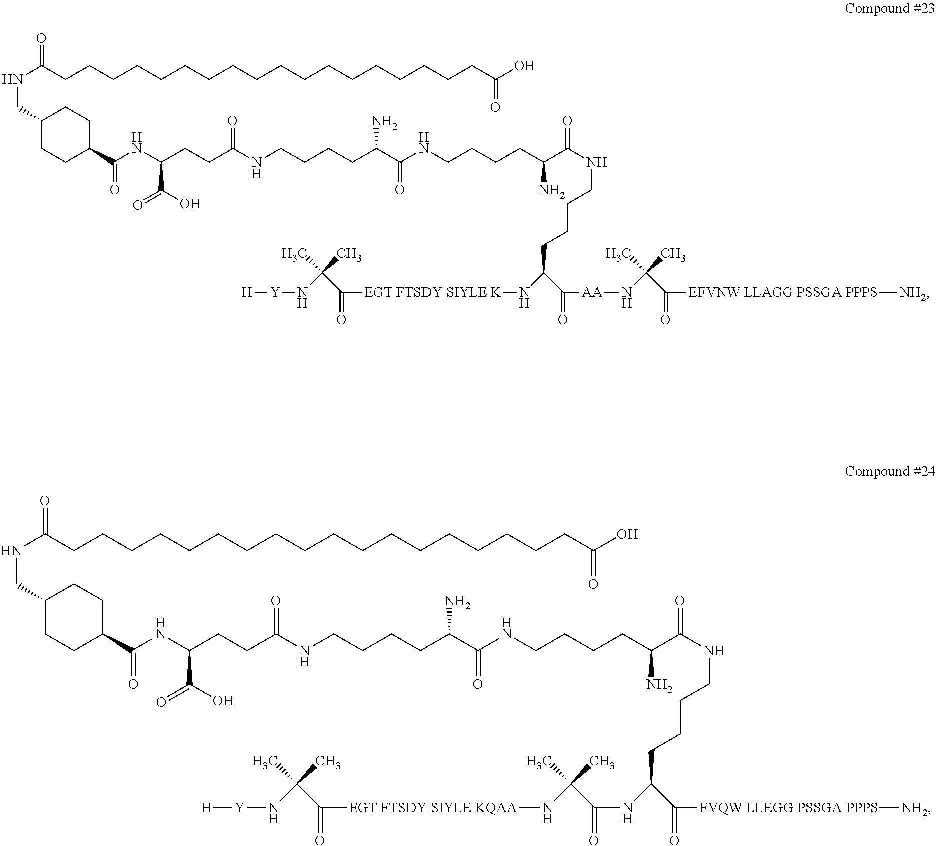

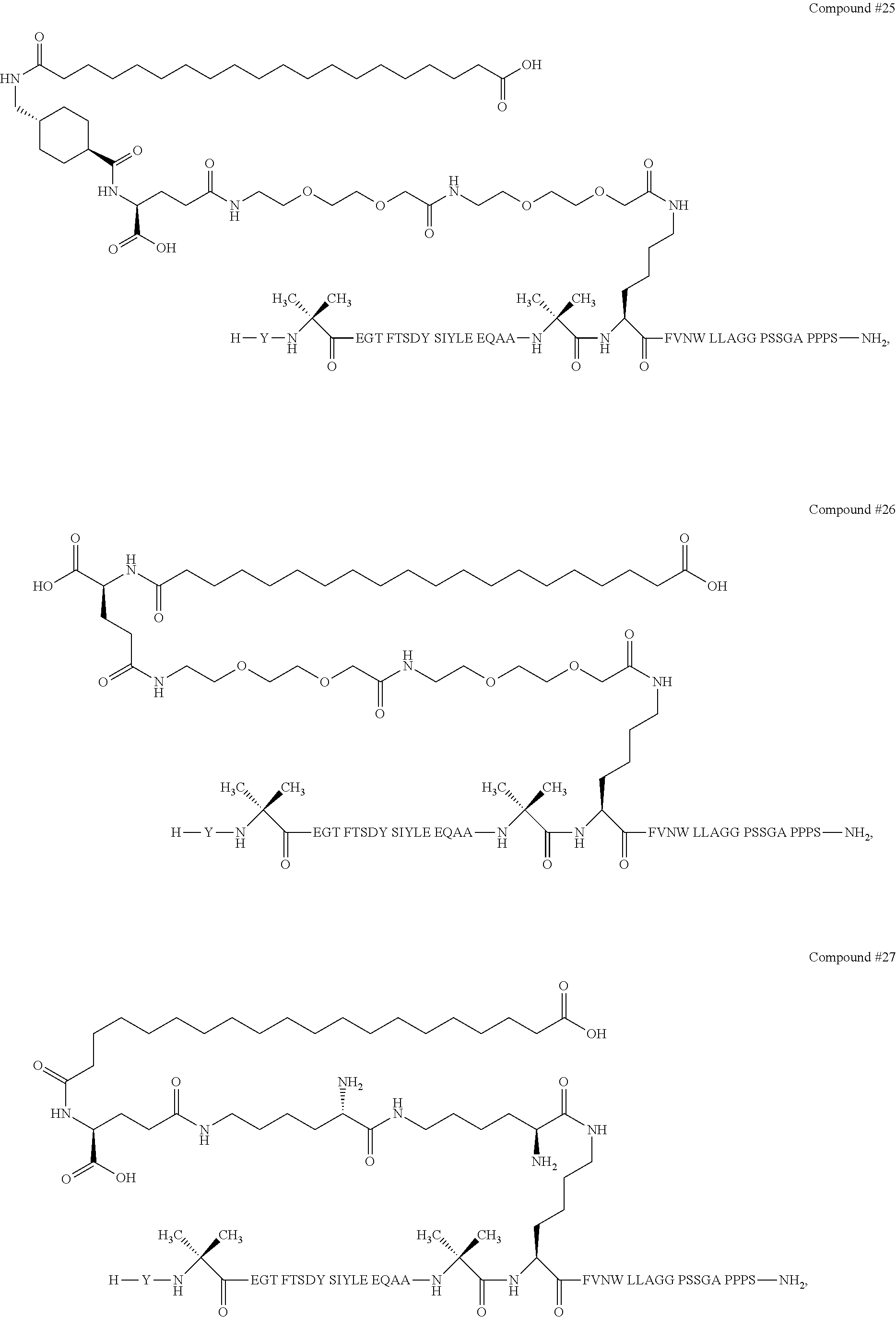

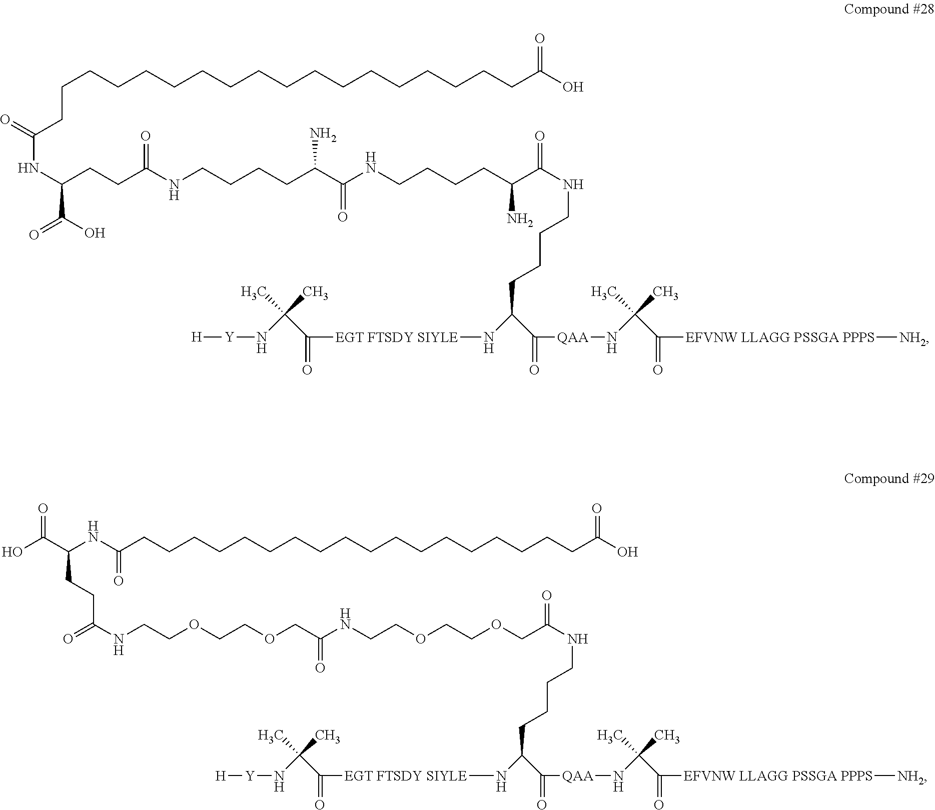

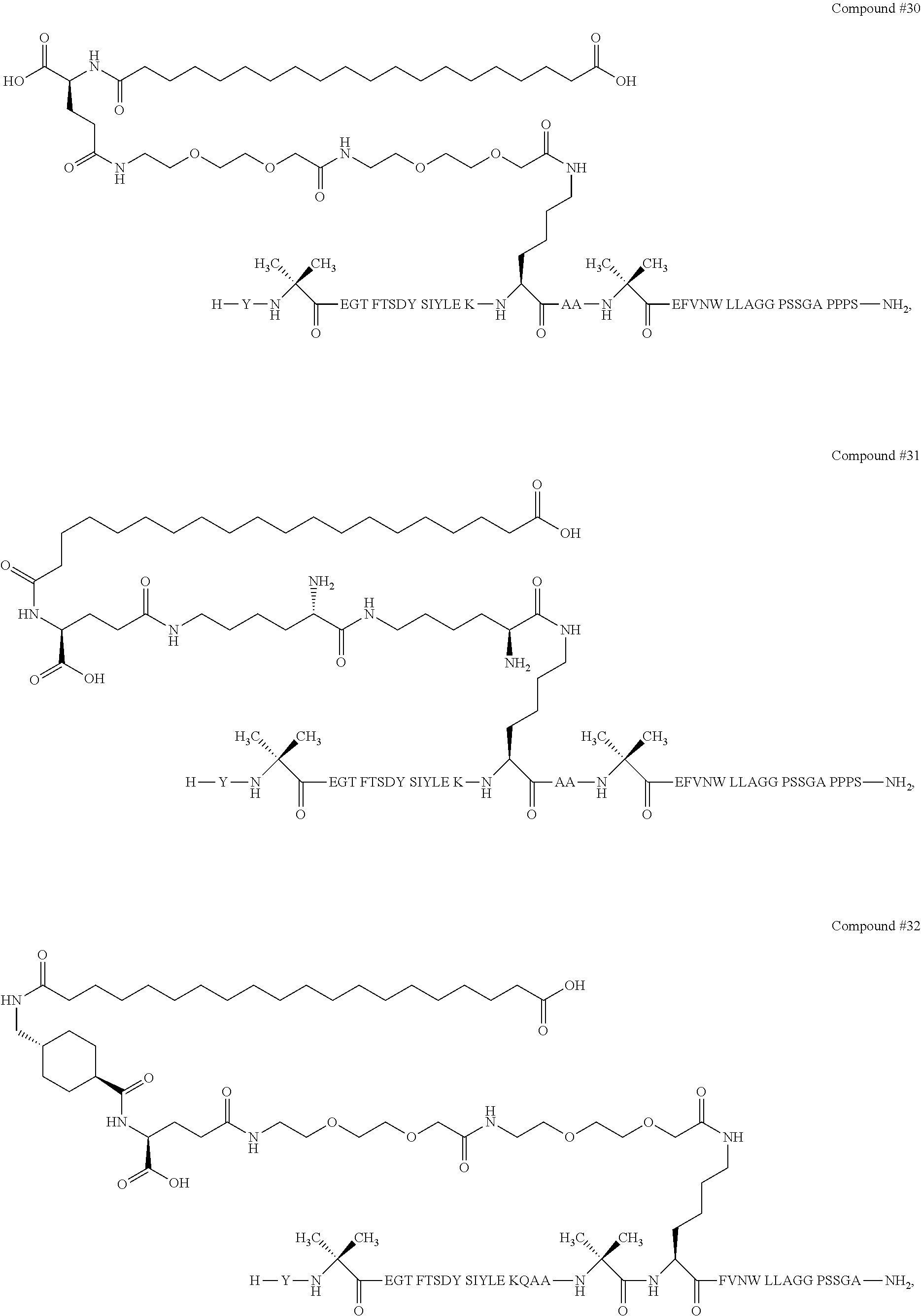

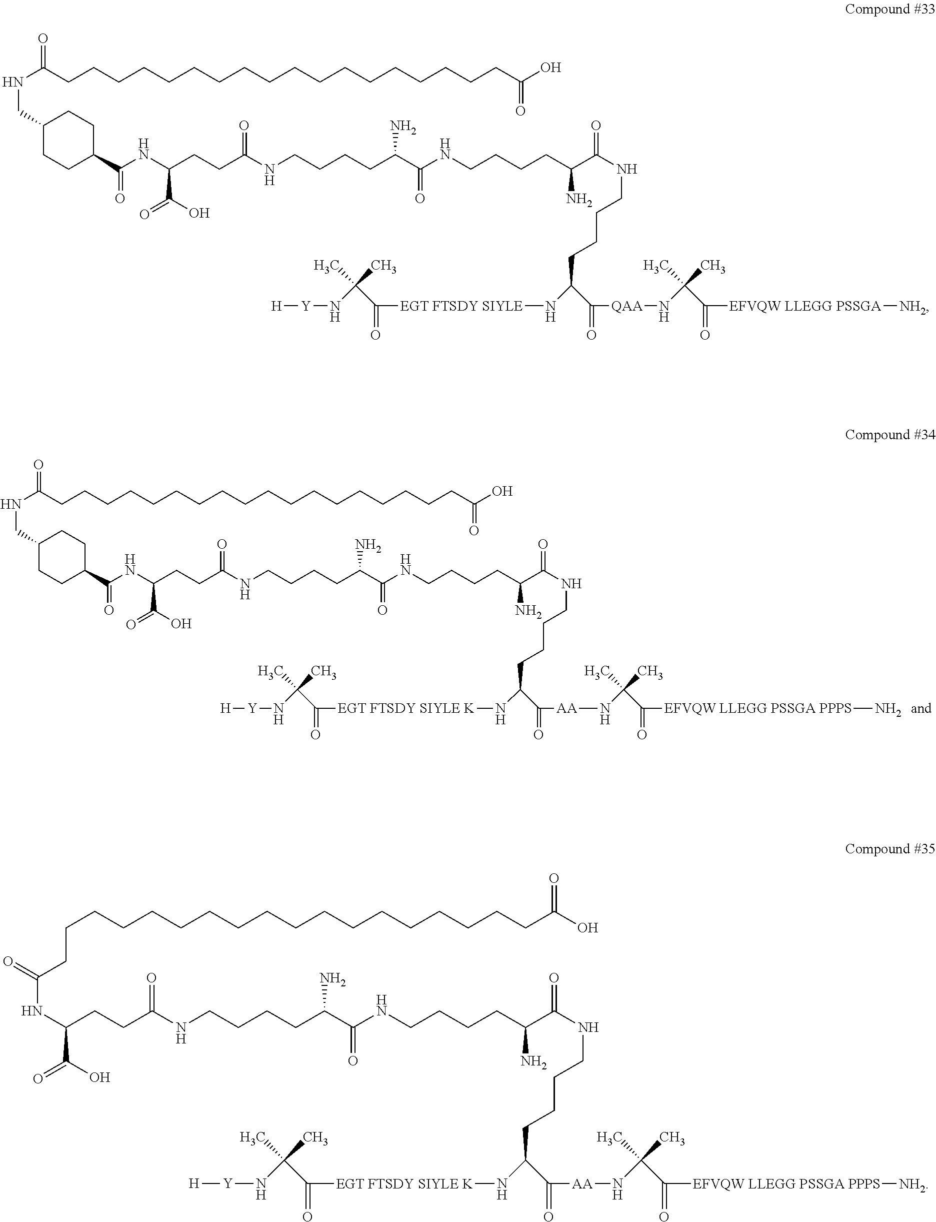

11. The compound according to claim 10, wherein the compound is

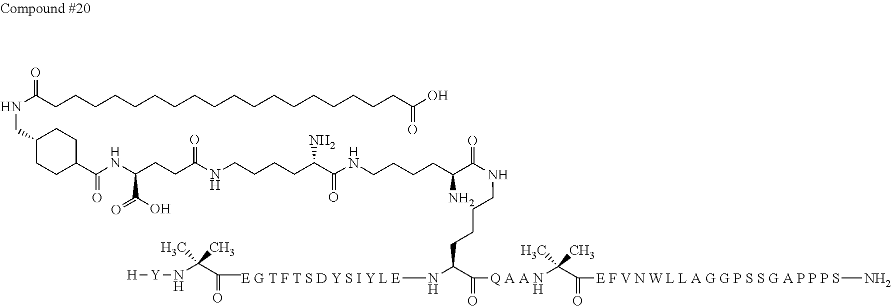

12.-13. (canceled)

14. A peptide having the amino acid sequence:
(SEQ ID NO.: 15)
YX₂EGTFTSDYSIYLX₁₅X₁₆X₁₇AAX₂₀X₂₁FVX₂₄WLLX₂₈GGPX₃₂X₃₃X₃₄X₃₅
X₃₆X₃₇X₃₈X₃₉,
wherein
X₂ is Aib,
X₁₅ is E,
X₁₆ is E or K,
X₁₇ is Q or K,
X₂₀ is Aib,
X₂₁ is E or K,
X₂₄ is N or Q,
X₂₈ is A or E,
X₃₂ is S or absent,
X₃₃ is S or absent,
X₃₄ is G or absent,
X₃₅ is A or absent,
X₃₆ is P or absent,
X₃₇ is P or absent,
X₃₈ is P or absent, and
X₃₉ is S or absent.

15. The peptide according to claim 14, wherein the amino acid sequence of the peptide is selected from the group consisting of SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, and SEQ ID NO.: 14.

16. The peptide according to claim 15, wherein the amino acid sequence of the peptide is SEQ ID NO.: 10.

17. The compound according to claim 1, further comprising an amide modification of the C-terminus.

18. The compound according to claim 6, further comprising an amide modification of the C-terminus.

19. The compound according to claim 7, further comprising an amide modification of the C-terminus.

20. The compound according to claim 6, wherein the substituent is attached at position 16.

21. The compound according to claim 7, wherein the substituent is attached at position 16.

22. The peptide according to claim 14, further comprising an amid modification of the C-terminus.

23. The peptide according to claim 15, further comprising an amid modification of the C-terminus.

24. The peptide according to claim 16, further comprising an amid modification of the C-terminus.

25. A method of reducing body weight, comprising administering the compound according to claim 1 to a patient in need thereof.

26. The method according to claim 25, wherein the amino acid sequence of the peptide is
(SEQ ID NO.: 16)
YX₂EGTFTSDYSIYLX₁₅X₁₆X₁₇AAX₂₀X₂₁FVX₂₄WLLX₂₈GGPSSGA;
and wherein
X₂ is Aib,
X₁₅ is D or E,
X₁₆ is E or K,
X₁₇ is Q or K,
X₂₀ is Aib,
X₂₁ is E or K,
X₂₄ is N or Q, and
X₂₈ is A or E.

27. The method according to claim 25, wherein the amino acid sequence of the peptide is selected from the group consisting of SEQ ID NO.: 2, SEQ ID NO.: 3, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, and SEQ ID NO.: 14.

28. The method according to claim 27, wherein the amino acid sequence of the peptide is SEQ ID NO.: 10.

29. The method according to claim 25, further comprising an amide modification of the C-terminus.

30. The method according to claim 27, further comprising an amide modification of the C-terminus.

31. The method according to claim 28, further comprising an amide modification of the C-terminus.

32. The method according to claim 25, wherein the substituent is attached at position 16.

33. The method according to claim 27, wherein the substituent is attached at position 16.

34. The method according to claim 28, wherein the substituent is attached at position 16.

35. The method according to claim 25, wherein the compound is selected from the group consisting of:

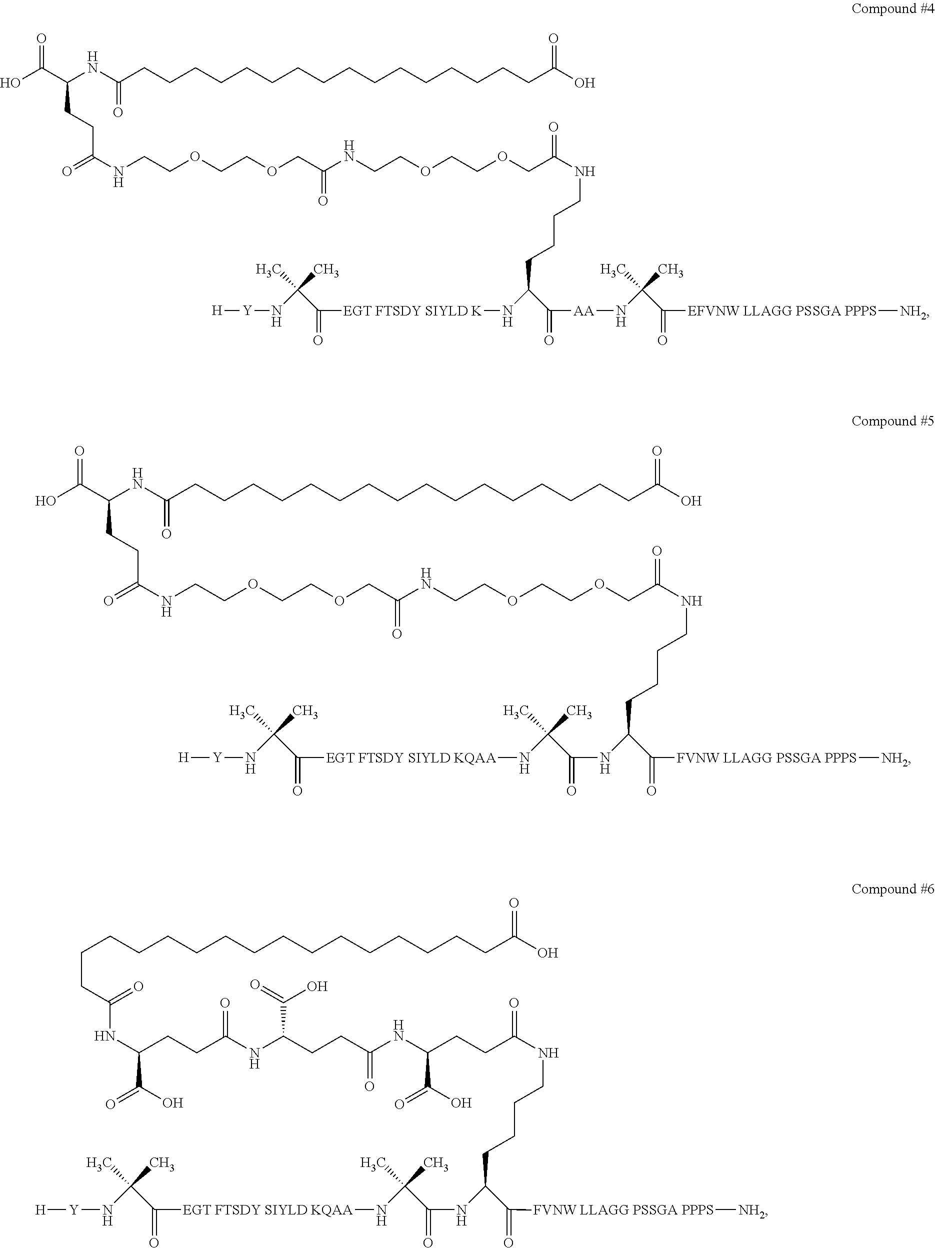

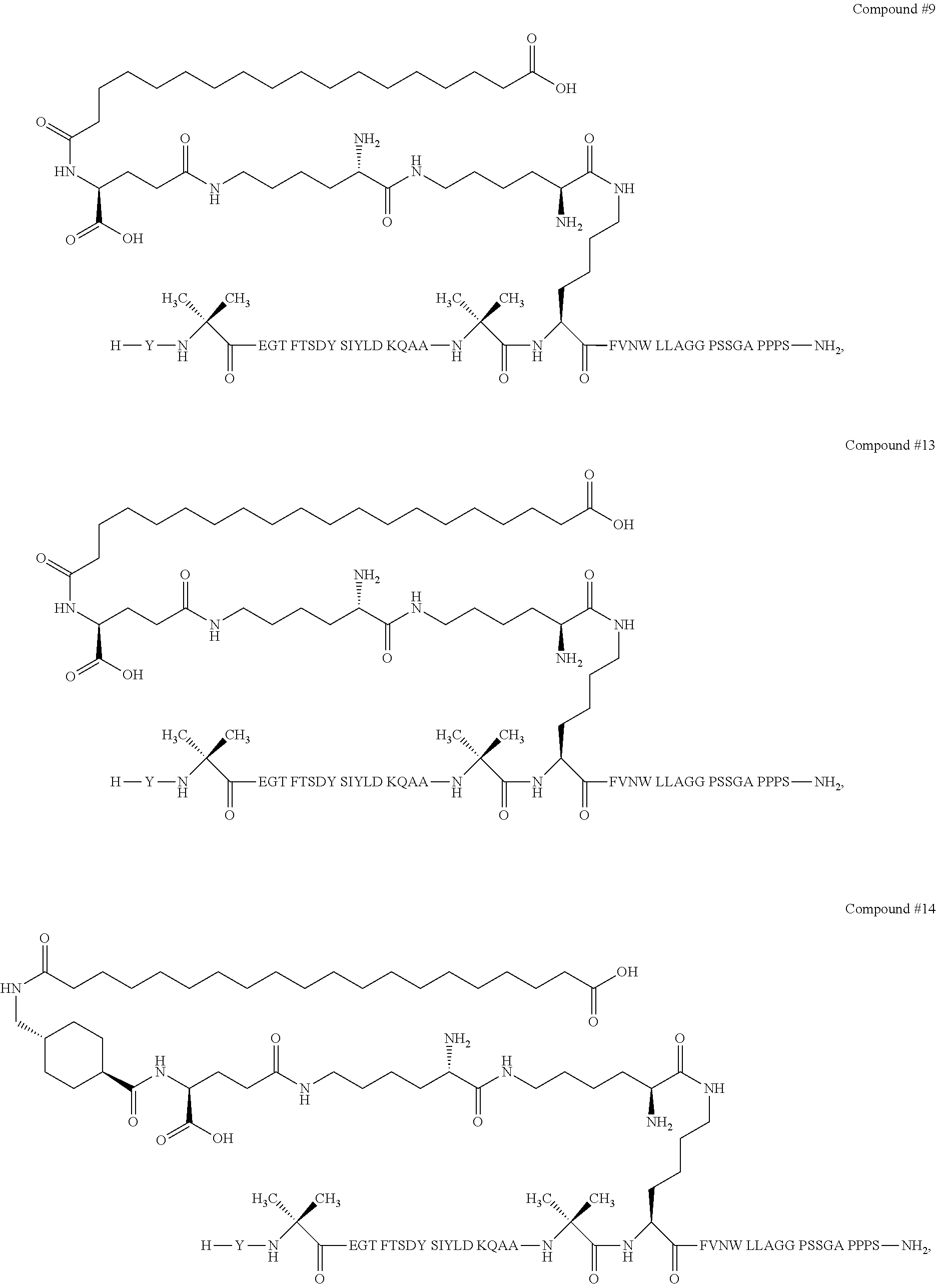

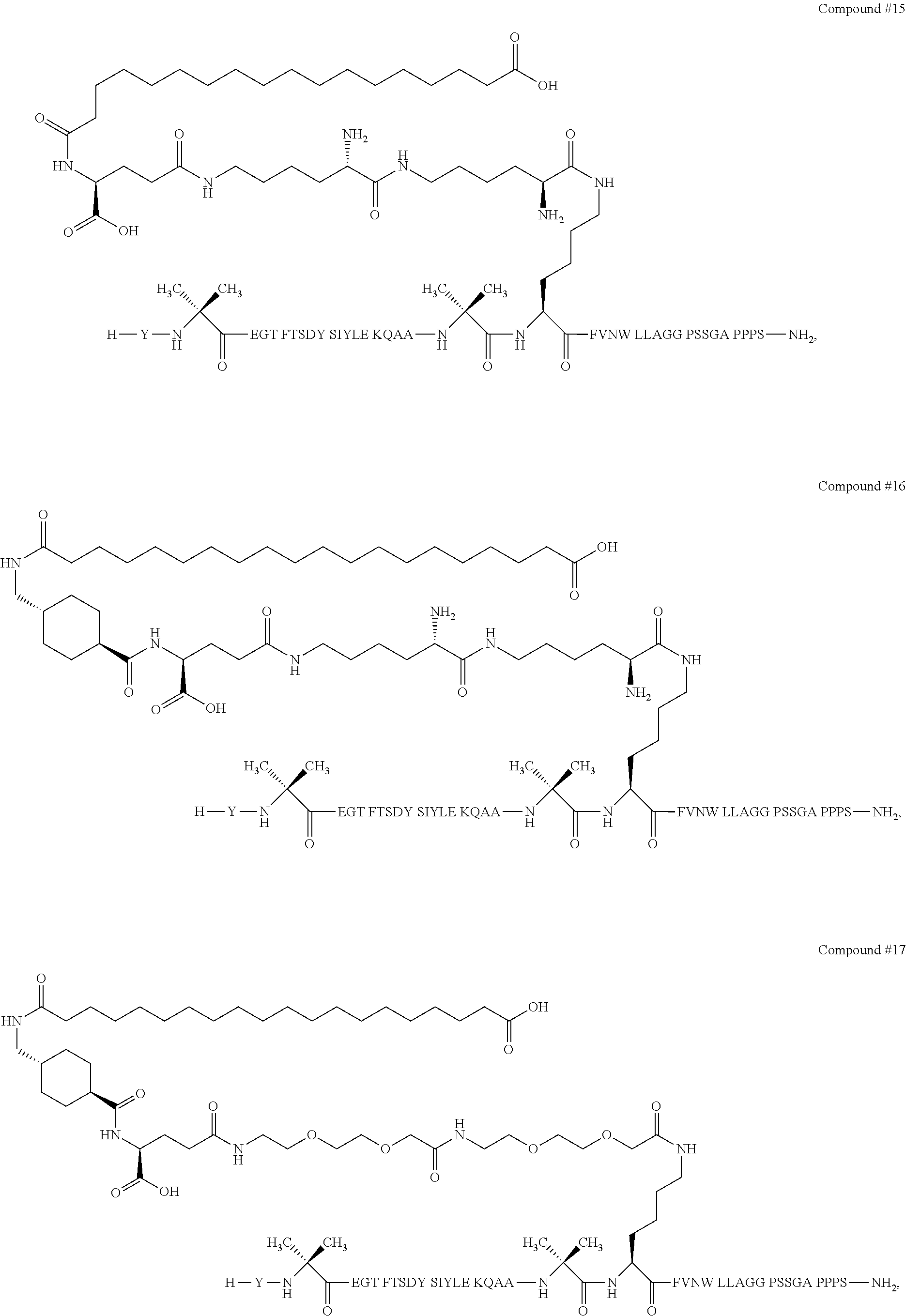

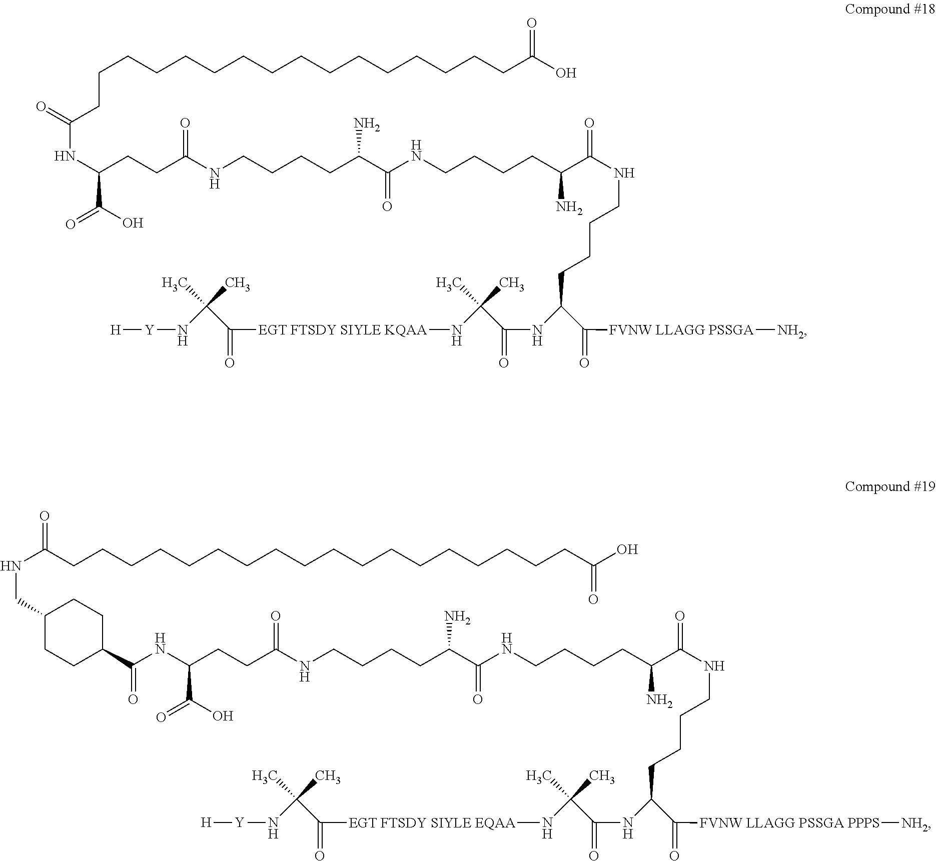

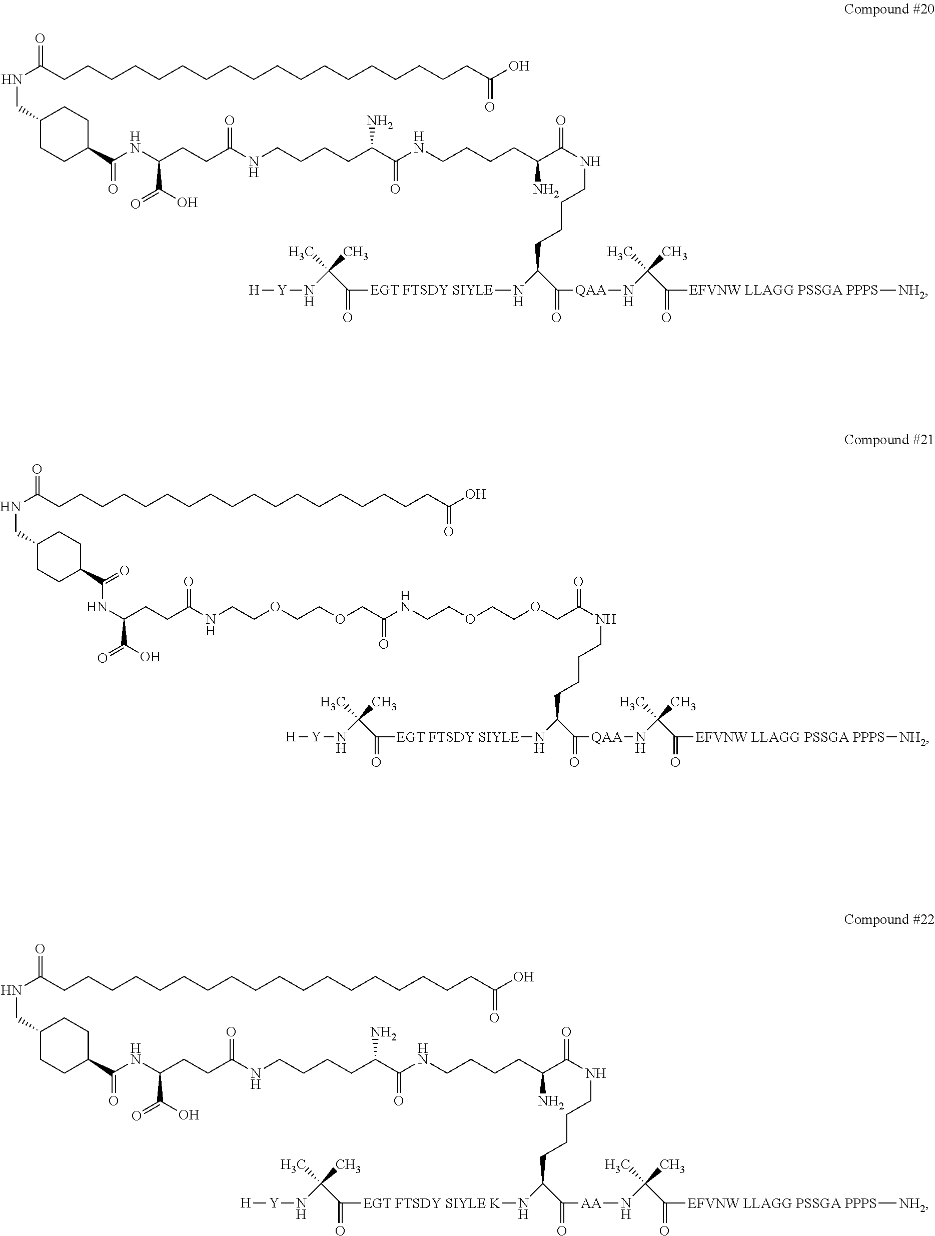

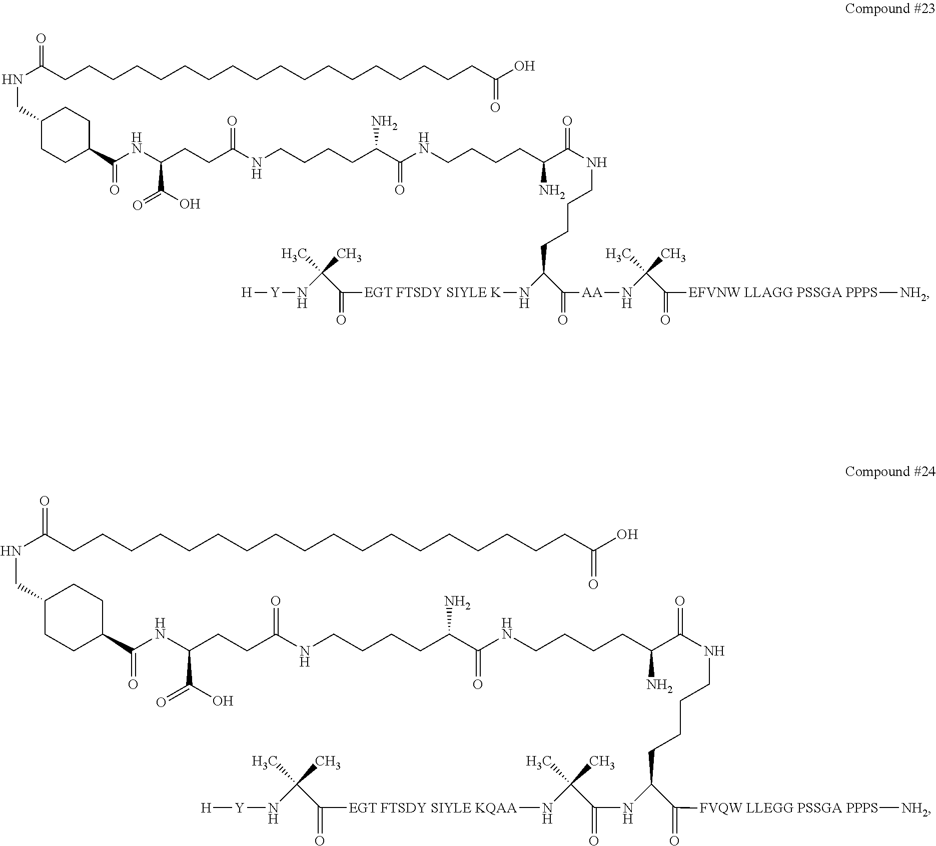

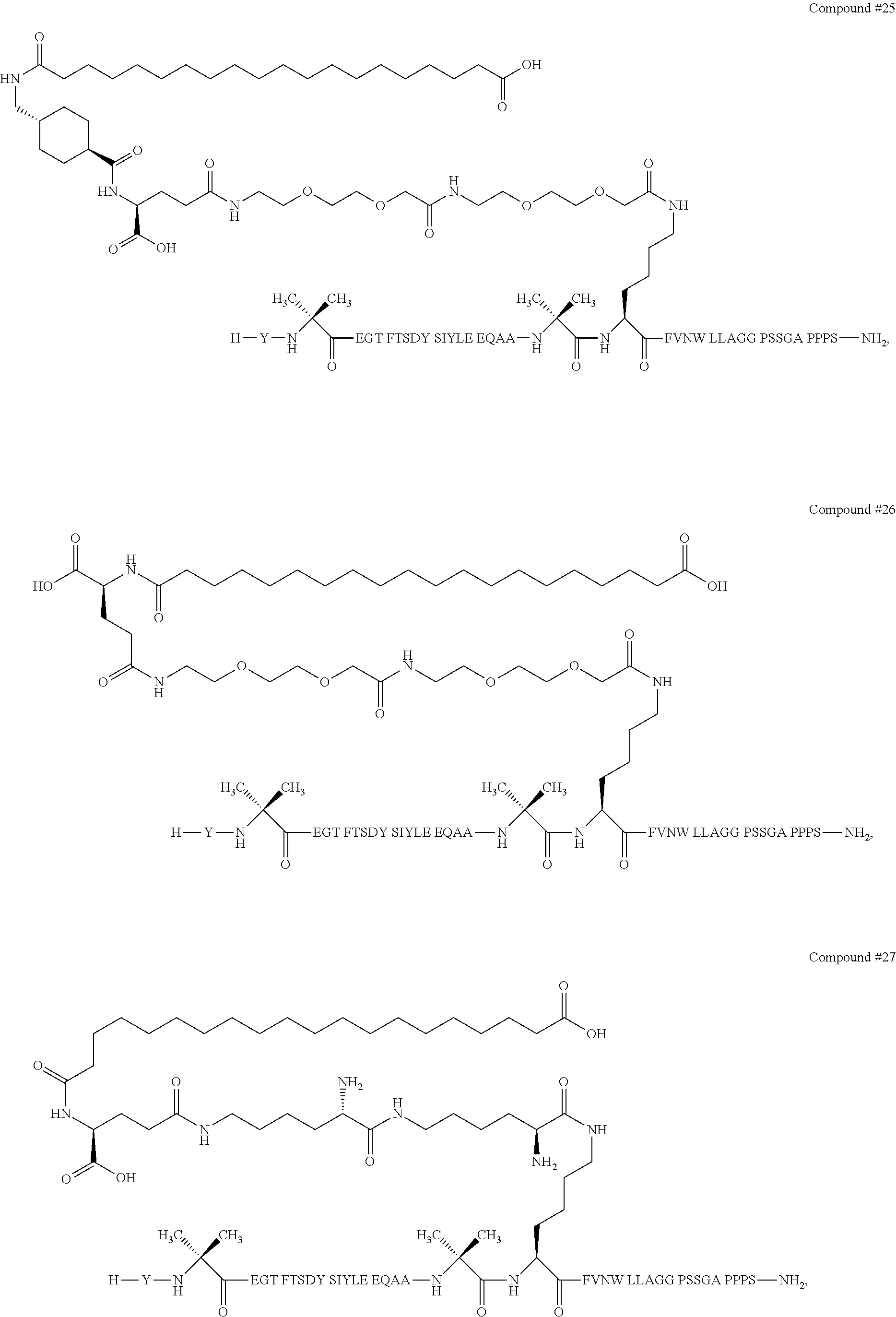

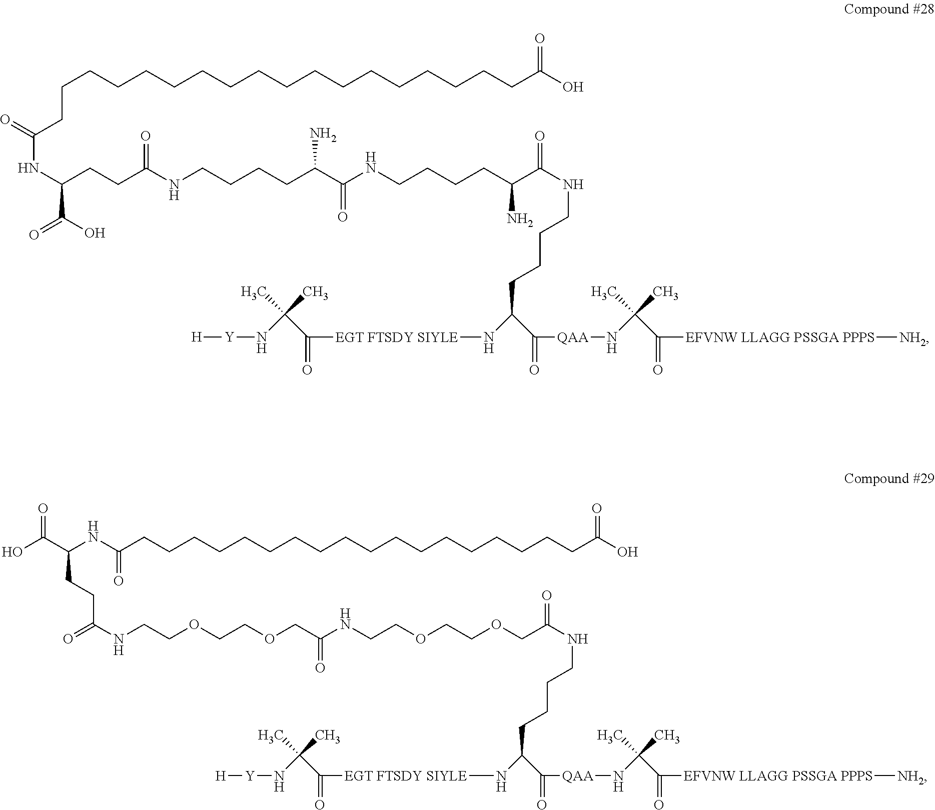

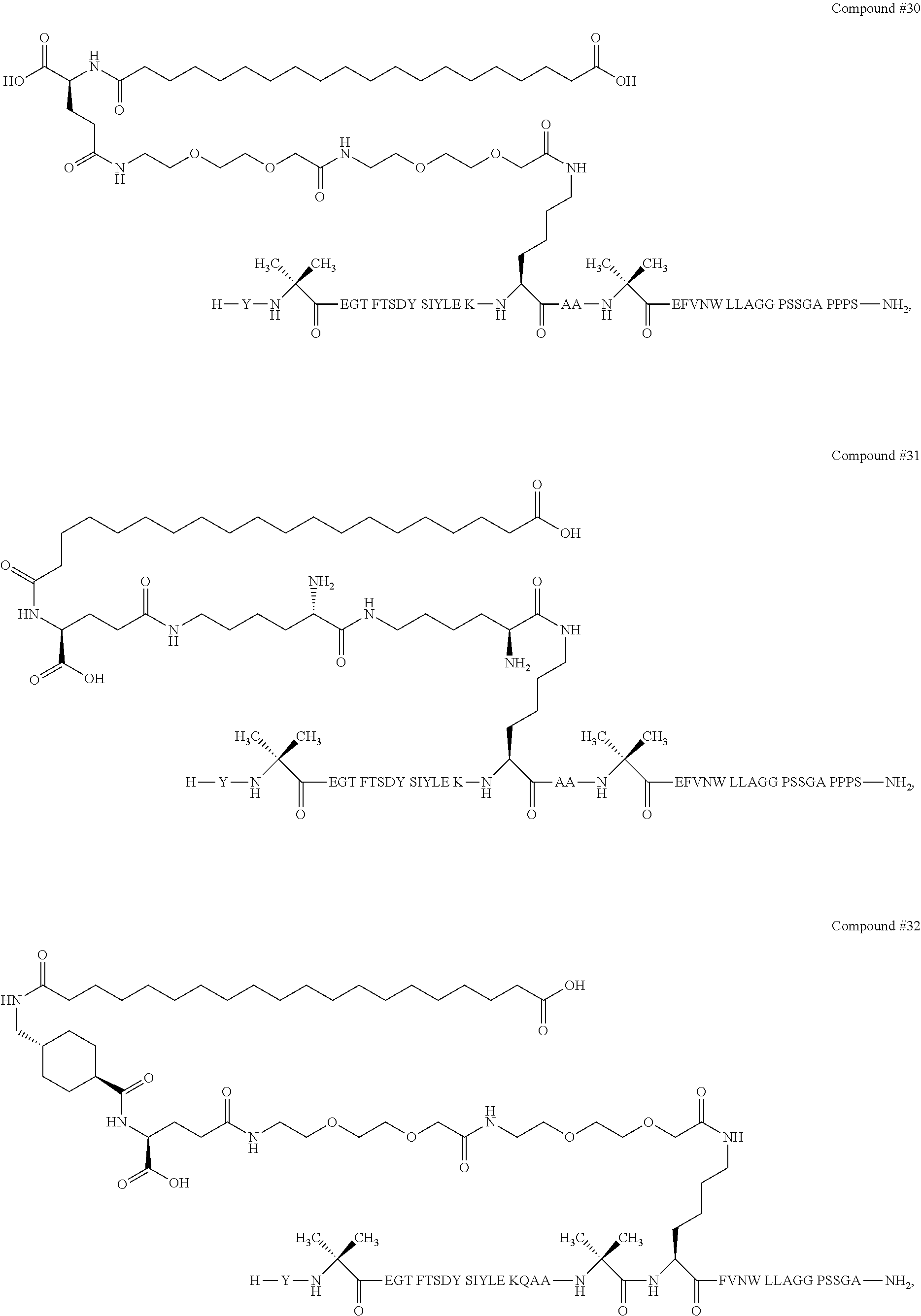

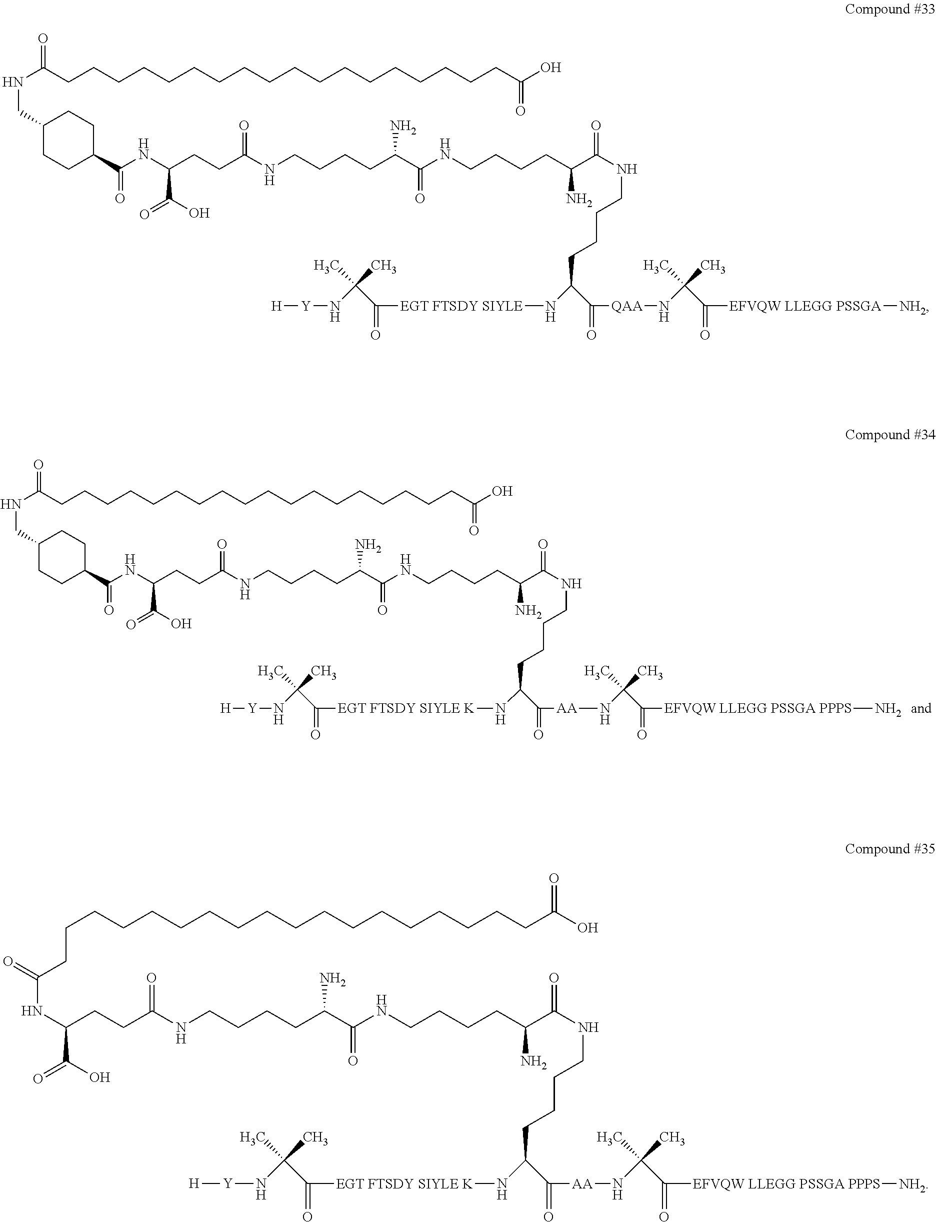

36. The compound according to claim 35, wherein the compound is [chemical structure]
